(19) United States
(12) Patent Application Publication
(10) Pub. No.: US 2007/0292866 A1
(43) Pub. Date: Dec. 20, 2007

(54) Diagnosing human diseases by detecting DNA methylation changes

(75) Inventors: Huajan Wang, Newton, MA (US); Anthony Anisowicz, West Newton, MA (US); Richard Del Mastro, Norfolk, MA (US); Hui Huang, Lexington, MA (US); Sergey Mamaev, West Roxbury, MA (US)

Correspondence Address: Peter G. Carroll;MEDLEN & CARROLL, LLP, Suite 350, 101 Howard Street, San Francisco, CA, 94105, US

(73) Assignee: AMBERGEN, INC.

(21) Appl. No.: 11/634,755

(22) Filed: Dec. 6, 2006

Related U.S. Application Data
(60) Provisional application No. 60/748,370, filed on Dec. 8, 2005.

(51) Int. Cl. C12Q 1/68

(52) U.S. Cl. 435/6

(57) ABSTRACT

This invention relates to methodologies that detect global changes in the methylation of human genomic DNA as well as changes in methylation in specific regions of the human genome. The methodologies have utility in the diagnosis, prognosis and monitoring of therapeutic treatment for any human disease. Further, the invention relates to methodologies that can detect global changes in the methylation of human genomic DNA that is a consequence of diet and/or dietary supplements. The invention also relates to identifying novel DNA methylation biomarkers that are associated with human disease.

### FIELD OF THE INVENTION

The present invention relates to compositions and methods of detecting changes in DNA methylation patterns. In one embodiment, DNA methylation patterns are detected by ligating a DNA fragment before digestion with a methylation insensitive restriction enzyme and/or a methylation sensitive restriction enzyme. In another embodiment, DNA methylation biomarkers are identified using primer pairs selective for a CpG Island. Such changes in DNA methylation patterns may provide disease diagnosis, prognosis, and potential therapeutics as well as determining general health.

### BACKGROUND

Many diseases are known to have inheritable traits. Blood diseases, for example, sickle cell anemia and hemophilia, were identified many years ago as being confined to specific families having common ancestry. Other diseases appear to have genetically-based causes, but the identification of specific genetic mutations or other inheritable regulatory disorders have eluded the medical arts. Cancer, for example, may be explained by many other factors besides genetic origins. These alternative potential origins may include environmental pollution and virus infection however, the origin of very few types of cancer have been positively identified.

Biochemical and physiological pathway regulation is complex and not well understood. Gene regulation is known to play a significant role in the expression of genes encoding regulatory enzymes controlling such pathways. Most studies, however, are limited to an effort in finding specific genetic mutations in the nucleic acid sequences of these genes. Ongoing research has failed to identify genomic regulatory mechanisms that are not genetically based. The identification and correction of non-genetically based transcriptional control mechanisms may help explain the inability to identify specific genetic mutations for known inheritable diseases.

What is needed in the art is a simple, fast, and economic assay that can be performed in a physician's office or hospital laboratory that can identify epigenetic alterations responsible for inheritable disease.

### SUMMARY OF THE INVENTION

The present invention relates to compositions and methods of detecting changes in DNA methylation patterns. In one embodiment, DNA methylation patterns are detected by ligating a DNA fragment before digestion with a methylation insensitive restriction enzyme and/or a methylation sensitive restriction enzyme. In another embodiment, DNA methylation biomarkers are identified using primer pairs selective for a CpG Island. Such changes in DNA methylation patterns may provide disease diagnosis, prognosis, and potential therapeutics as well as determining general health.

In one embodiment, the present invention contemplates a forward primer comprising a nucleic acid sequence that is complementary to a 5′ CpG Island boundary, wherein said boundary comprises a methylation restriction site. In one embodiment, the sequence comprises at least six nucleic acids. In one embodiment, the sequence comprises less than eight guanosine nucleotides. In one embodiment, the sequence comprises less than eight cytosine nucleotides.

In one embodiment, the present invention contemplates a CpG Island Primer construction method comprising, a) providing, i) a genomic sequence comprising at least one specific nucleotide window, ii) a computer program, wherein said program is capable of scanning said genomic sequence for said specific nucleotide window, iii) a CpG report program that is capable of identifying CpG nucleotide boundaries within said genomic sequence; b) determining said CpG nucleotide boundaries within said genomic sequence, wherein said boundaries comprise a 5′ CpG boundary and a 3′ CpG boundary; and c) calculating a specific nucleotide window frequency within said CpG Island sequence, wherein said 5′ CpG boundary comprises a methylation restriction site. In one embodiment, the method further comprises synthesizing a complementary nucleotide sequence to said 5′ CpG boundary to create a forward primer. In one embodiment, the forward primer comprises at least six nucleotide sequences. In one embodiment, the forward primer comprises less than eight guanosine nucleotides. In one embodiment, the forward primer comprises less than eight cytosine nucleotides.

In one embodiment, the present invention contemplates a composition comprising a nucleic acid between at least nine and twenty nucleic acids having: i) at least one CG dinucleotide; ii) Z as any nucleic acid or nothing; iii) X and Y as different nucleic acids, and wherein said nucleic acid contains fewer than seven cytosines, and fewer than seven guanosines. In one embodiment, the composition is Z-C-G-Xₙ—Yᵣ, wherein n and r are independently whole numbers between 2 and 5. In one embodiment, the composition is Z-C-G-Xₙ-Z-Yᵣ, wherein n and r are independently whole numbers between 2 and 5. In one embodiment, the composition is Z-C-G-Z-Xₙ—Yᵣ, wherein n and r are independently whole numbers between 2 and 5. In one embodiment, the composition is Z-C-G-Xₙ—Yᵣ-Z wherein n and r are independently whole numbers between 2 and 5. In one embodiment, the composition is Xₙ-Z-C-G-Yᵣ wherein n and r are independently whole numbers between 2 and 6. In one embodiment, the composition is Xₙ—Yᵣ-Z-C-G-Xₙ—Yᵣ, wherein n and r are independently whole numbers between 1 and 3. In one embodiment, the composition is Xₙ-Z-Yᵣ—C-G-Xₙ-Z-Yᵣ, wherein n and r are independently whole numbers between 1 and 3. In one embodiment, the composition is Z_{q}-Xₙ—Yᵣ—C-G-Z_{q}-Xₙ—Yᵣ, wherein q, n and r are independently whole numbers between 0 and 3. In one embodiment, the composition is Z_{q}-Xₙ—Yᵣ—C-G-Z_{q}, wherein q, n and r are independently whole numbers between 0 and 3. In one embodiment, the composition is a 10-mer and comprises methylation sensitive restriction site. In one embodiment, the composition is selected from the forward and reverse primers of Table 1.

In one embodiment, the present invention contemplates a method, comprising: a) providing; i) a nucleic acid comprising at least one CG dinucleotide, wherein the cytosine is methylated; ii) a primer set comprising a forward primer and a reverse primer, wherein said forward primer will hybridize to a region of said nucleic acid comprising said CG dinucleotide; iii) a methylation specific restriction enzyme capable of cleaving a methylation restriction site provided said site is non-methylated; b) treating said nucleic acid with said methylation specific restriction enzyme so as to create a digest comprising fragments, wherein at least one of said fragments comprises said region comprising said CG dinucleotide; and c) introducing said forward and reverse primers under conditions such that said forward primer hybridizes to said region comprising CG dinucleotide and a portion of said fragment is amplified so as to create amplified product. In one embodiment, the forward primer is of the formula: Z-C-G-Xₙ—Yᵣ, wherein X and Y are different nucleic acid bases, wherein Z is any nucleic acid base or nothing, and wherein n and r are independently whole numbers between 2 and 5, wherein the primer as a whole has fewer than seven cytosines and fewer than seven guanosines. In one embodiment, the forward primer is of the formula: Z-C-G-Xₙ-Z-Yᵣ, wherein X and Y are different nucleic acid bases, wherein Z is any nucleic acid base or nothing, and wherein n and r are independently whole numbers between 2 and 5, wherein the primer as a whole has fewer than seven cytosines and fewer than seven guanosines. In one embodiment, the forward primer is of the formula: Z-C-G-Z-Xₙ—Yᵣ, wherein X and Y are different nucleic acid bases, wherein Z is any nucleic acid base or nothing, and wherein n and r are independently whole numbers between 2 and 5, wherein the primer as a whole has fewer than seven cytosines and fewer than seven guanosines. In one embodiment, the forward primer is of the formula: Z-C-G-Xₙ—Yᵣ-Z wherein X and Y are different nucleic acid bases, wherein Z is any nucleic acid base or nothing, and wherein n and r are independently whole numbers between 2 and 5, wherein the primer as a whole has fewer than seven cytosines and fewer than seven guanosines. In one embodiment, the forward primer is of the formula: Xₙ—C-G-Yᵣ, wherein X and Y are different nucleic acid bases and wherein n and r are independently whole numbers between 2 and 6, wherein the primer as a whole has fewer than seven cytosines and fewer than seven guanosines. In one embodiment, the forward primer is of the formula: Xₙ—Yᵣ—C-G-Xₙ—Yᵣ, wherein X and Y are different nucleic acid bases and n and r are independently whole numbers between 1 and 3, wherein the primer as a whole has fewer than seven cytosines and fewer than seven guanosines. In one embodiment, the forward primer is of the formula: Xₙ-Z-Yᵣ—C-G-Xₙ-Z-Yᵣ, wherein X and Y are different nucleic acid bases, wherein Z is any nucleic acid base or nothing, and n and r are independently whole numbers between 1 and 3, wherein the primer as a whole has fewer than seven cytosines and fewer than seven guanosines. In one embodiment, the forward primer is of the formula: Z_{q}-Xₙ—Yᵣ—C-G-Z_{q}-Xₙ—Yᵣ, wherein X and Y are different nucleic acid bases, wherein Z is any nucleic acid base, and wherein q, n and r are independently whole numbers between 0 and 3, wherein the primer as a whole has fewer than seven cytosines and fewer than seven guanosines. In one embodiment, the forward primer is of the formula: Z_{q}-Xₙ—Yᵣ—C-G-Z_{q}, wherein X and Y are different nucleic acid bases, wherein Z is any nucleic acid base, and wherein q, n and r are independently whole numbers between 0 and 3, wherein the primer as a whole has fewer than seven cytosines and fewer than seven guanosines. In one embodiment, the forward primer is a 10-mer and comprises methylation sensitive restriction site. In one embodiment, the forward primer and said reverse primer is selected from the forward and reverse primers of Table 1.

In one embodiment, the present invention contemplates a GMSP/MFSP method, comprising: a) providing; i) a nucleic acid comprising at least one 5′ CpG Island boundary, wherein said boundary comprises at least one methylation restriction site; ii) at least one primer set comprising a forward primer and a reverse primer, wherein said forward primer is substantially homologous to said restriction site; iii) a methylation specific restriction enzyme capable of cleaving said methylation restriction site provided said site is methylated; iv) a methylation sensitive restriction site capable of cleaving said methylation restriction site provided said site is non-methylated; iii) a methylation insensitive restriction enzyme capable of cleaving said restriction site whether said restriction site is methylated or non-methylated; b) contacting a first aliquot of said DNA with said methylation specific restriction enzyme to generate a first DNA fragment that is substantially homologous to said primer set; c) contacting a second aliquot of said DNA with said methylation sensitive restriction enzyme to generate a second DNA fragment that is substantially homologous to said primer set; d) contacting a third aliquot of said DNA with said methylation insensitive restriction enzyme to generate a third DNA fragment that is substantially homologous to said primer set. In one embodiment, the first, second and third DNA fragments are end-labeled. In one embodiment, the method further comprises step c) amplifying said first, second and third DNA fragments to generate cDNA. In one embodiment, the method further comprises step d) separating said cDNA under conditions such that said methylation restriction site is identified. In one embodiment, the nucleic acid is selected from the group consisting of free circulating DNA and genomic DNA.

In one embodiment, the present invention contemplates a GM/RESA method, comprising: a) providing, i) isolated genomic DNA comprising a first DNA aliquot and a second DNA aliquot, wherein said DNA comprises at least one 5′ CpG Island boundary, wherein said boundary comprises a methylated restriction site; ii) a methylation sensitive restriction enzyme; iii) a methylation insensitive restriction enzyme; and iv) a forward primer, wherein said primer has substantial homology to said 5′ CpG Island boundry; b) contacting said first DNA aliquot with said methylation sensitive restriction enzyme to create a first digest; c) contacting said second DNA aliquot with said methylation insensitive restriction enzyme to create a second digest; d) hybridizing said forward primer to said 5′ boundary under conditions such that said DNA is amplified; and e) detecting said amplified fragments under conditions such that said methylated restriction site is identified.

In one embodiment, the present invention contemplates a method, comprising: a) providing, i) genomic DNA; ii) a methylation sensitive restriction enzyme; iii) a methylation insensitive restriction enzyme; iv) all 4 dideoxynucleotides, v) at least one labeled deoxynucleotide; b) end filling said genomic DNA with said dideoxynucleotides to create end-filled DNA; c) contacting a first aliquot of said end-labeled DNA with said methylation sensitive restriction enzyme to create a first digest comprising DNA fragments; d) contacting a second aliquot of said end-labeled DNA with said methylation insensitive restriction enzyme to create a second digest comprising DNA fragments; e) treating separately said first and second digest so as to introduce at least one labeled deoxynucleotide into at least a portion of said fragments, thereby creating two populations of labeled fragments; f) immobilizing at least a portion of each of said two populations of labeled fragments; and g) detecting said label. In one embodiment, the label is biotin. In one embodiment, two biotin labeled deoxynucleotides are utilized in step e). In one embodiment, one of said labeled deoxynucleotide is biotin labeled dCTP. In one embodiment, one of said labeled deoxynucleotide is biotin labeled dGTP. In one embodiment, the two biotin labeled deoxynucleotides are biotin labeled dCTP and biotin labeled dGTP. In one embodiment, the biotin is quantitated in step g). In one embodiment, the methylation sensitive restriction enzyme is selected from the group consisting of Hpa 1 and BssH11. In one embodiment, the methylation is sensitive restriction enzyme is Msp1. In one embodiment, the end filling of step b) is performed with T7 DNA polymerase. In one embodiment, the treating of step e) is performed with T7 DNA polymerase. In one embodiment, the genomic DNA is obtained from a cancer cell.

In one embodiment, the present invention contemplates an MSRquant method, comprising: a) providing, i) freely circulating DNA isolated from a biological sample capable of end-label ligation, wherein said DNA comprises at least one methylated restriction site; ii) a double stranded oligonucleotide linker capable of end-labeling said DNA, wherein said linker comprises a portion that is not homologous with said DNA; iii) a primer having substantial homology to said linker portion; iv) a methylation sensitive restriction enzyme; and v) a methylation insensitive restriction enzyme; b) ligating said DNA with said linker, wherein said ligation comprises an end-labeled DNA; c) contacting a first end-labeled DNA aliquot with said methylation sensitive restriction enzyme to create a first digest; and d) contacting a second end-labeled DNA aliquot with said methylation insensitive restriction enzyme to create a second digest. In one embodiment, the method further comprises step d) hybridizing said primer to both first and second digests. In one embodiment, the method further comprises step d) amplifying said hybridized digests to create cDNA. In one embodiment, the method further comprises step e) isolating said cDNA under conditions such that said restriction sites are identified. In one embodiment, the biological sample is selected from the group comprising tissue samples, blood samples, stool samples, spinal fluid samples, saliva samples, urine samples, buccal samples, or any other bodily fluid samples.

In one embodiment, the present invention contemplates an MSRquant method, comprising: a) providing, i) genomic DNA; ii) a double stranded oligonucleotide linker, wherein said linker comprises a portion that is not homologous with said genomic DNA; iii) a primer having substantial homology to said portion of said linker; iv) a methylation sensitive restriction enzyme; v) a probe; and vi) a methylation insensitive restriction enzyme; b) ligating said linker to said genomic DNA to create end-labeled DNA; c) contacting a first aliquot of said end-labeled DNA with said methylation sensitive restriction enzyme to create a first digest; d) contacting a second aliquot of said end-labeled DNA with said methylation insensitive restriction enzyme to create a second digest; e) introducing said primer to said first digest under conditions such that first amplified product is generated; f) introducing said primer to said second digest under conditions such that a second amplified product is generated; and g) hybridizing said probe to said first and second amplified products. In one embodiment, the method further comprises, prior to the ligation of step b), said genomic DNA is treated to create blunt end fragments. In one embodiment, the genomic DNA is isolated from a biological sample selected from the group comprising tissue samples, blood samples, stool samples, spinal fluid samples, saliva samples, urine samples, buccal samples, or any other bodily fluid samples. In one embodiment, the genomic DNA is free circulating DNA isolated from plasma. In one embodiment, the probe is designed to hybridize to a DNA methylation biomarker associated with a disease. In one embodiment, the hybridizing in step (g) to said first and second amplified products is performed in separate reactions. In one embodiment, the probe is labeled. In one embodiment, the probe is immobilized prior to said hybridizing in step (g). In one embodiment, the first and second amplified products are immobilized in separate regions of a surface.

In one embodiment, the present invention contemplates an MSRquant method, comprising: a) providing, i) genomic DNA; ii) a double stranded oligonucleotide linker, wherein said linker comprises a portion that is not homologous with said genomic DNA; iii) a primer having substantial homology to said portion of said linker; iv) a methylation sensitive restriction enzyme; v) a probe, wherein said probe is designed to hybridize to a DNA methylation biomarker associated with a disease; and vi) a methylation insensitive restriction enzyme; b) ligating said linker to said genomic DNA to create end-labeled DNA; c) contacting a first aliquot of said end-labeled DNA with said methylation sensitive restriction enzyme to create a first digest; d) contacting a second aliquot of said end-labeled DNA with said methylation insensitive restriction enzyme to create a second digest; e) introducing said primer to said first digest under conditions such that first amplified product is generated; f) introducing said primer to said second digest under conditions such that a second amplified product is generated; and g) hybridizing said probe to said first and second amplified products. In one embodiment, the method further comprises, prior to the ligation of step b), said genomic DNA is treated to create blunt end fragments. In one embodiment, the genomic DNA is isolated from a biological sample selected from the group comprising tissue samples, blood samples, stool samples, spinal fluid samples, saliva samples, urine samples, buccal samples, or any other bodily fluid samples. In one embodiment, the genomic DNA is free circulating DNA isolated from plasma. In one embodiment, the hybridizing in step (g) to said first and second amplified products is performed in separate reactions. In one embodiment, the probe is labeled. In one embodiment, the probe is immobilized prior to said hybridizing in step (g). In one embodiment, the first and second amplified products are immobilized in separate regions of a surface.

In one embodiment, the present invention contemplates a MESAS method, comprising: a) providing; i) isolated genomic DNA comprising at least one methylated restriction site; ii) a methylation specific restriction enzyme capable of cleaving said restriction site; iii) an end-labeling preparation comprising biotin-dCTP and biotin-dGTP, wherein said preparation is capable of end-labeling said restriction site; iv) a double stranded oligonucleotide linker capable of ligating with said end-labeled restriction site, wherein said linker comprises a portion that is not homology with said DNA; v) a first primer having substantial homology to said linker portion; and vi) a second primer having substantial homology to said restriction site; b) contacting said restriction enzyme with said DNA to create a digest; and c) contacting said digest with said end-labeling preparation to create an end-labeled preparation. In one embodiment, the methylation specific restriction enzyme is BisI. In one embodiment, the method further comprises step d) ligating said end-labeled preparation with said linker. In one embodiment, the further comprises step e) hybridizing said first and second primers under conditions such that said preparation is amplified. In one embodiment, the method further comprises step i) separating said amplified preparation under conditions such that said methylated restriction sites are identified. In one embodiment, the genomic DNA is derived from a diseased patient. In one embodiment, the genomic DNA is derived from a non-diseased patient. In one embodiment, comparing said methylated restriction sites between said diseased and non-diseased patients identifies a disease-specific methylated restriction site pattern.

In one embodiment, the present invention contemplates a method, comprising: a) providing, i) genomic DNA; ii) a double stranded oligonucleotide linker, wherein said linker comprises a portion that is not homologous with said genomic DNA; iii) a first primer having substantial homology to said portion of said linker; iv) an enzyme selected from the group comprising restriction enzymes that will cut at cytosines that have a methyl group and restriction enzymes that will not cut at cytosines that have a methyl group; b) contacting said genomic DNA with said enzyme to create a digest comprising fragments; c) ligating said linker to at least a portion of said fragments so as to create end-labeled DNA; d) introducing said primer to said digest under conditions such that amplified product is generated; and e) detecting said amplified product. In one embodiment, the method further comprises, prior to step b) said genomic DNA is treated to create blunt ends. In one embodiment, the method further comprises, prior to the ligation of step c) said digest is treated to create blunt end fragments. In one embodiment, the genomic DNA is isolated from a biological sample selected from the group comprising tissue samples, blood samples, stool samples, spinal fluid samples, saliva samples, urine samples, and buccal samples. In one embodiment, the method further comprises a second primer used in step (d). In one embodiment, the second primer comprises a region that is complimentary to a methylation sensitive restriction site. In one embodiment, the second primer further comprises degenerate bases. In one embodiment, the linker further comprises an EcoR1 restriction site. In one embodiment, the detecting of step e) comprises gel electrophoresis. In one embodiment, at least a portion of said amplified product is removed from the gel after electrophoresis to create isolated amplified product. In one embodiment, at least a portion of said isolated amplified product is introduced in a vector so as to create cloned fragments. In one embodiment, the vector is an EcoR1 linearized vector. In one embodiment, the vector is introduced into an E. coli host and said cloned fragments are propagated. In one embodiment, at least a portion of said cloned fragments are sequenced.

In one embodiment, the present invention contemplates a method, comprising: a) providing, i) first and second samples of DNA; ii) a double stranded oligonucleotide linker, wherein said linker comprises a portion that is not homologous with said genomic DNA; iii) a first primer having substantial homology to said portion of said linker; iv) a second primer comprising a region that is complimentary to a methylation restriction site; v) an enzyme selected from the group comprising restriction enzymes that will cut at cytosines that have a methyl group and restriction enzymes that will not cut at cytosines that have a methyl group; b) contacting in separate reactions said first and second samples of genomic DNA with said enzyme to create first and second digests comprising fragments; c) ligating said linker to at least a portion of said fragments in said first and second digests so as to create a first and second populations of end-labeled DNA; d) introducing said first and second primers to said first and second populations under conditions such that first and second amplified product is generated; and e) comparing said first and second amplified product. In one embodiment, the first sample of DNA is from a normal human free of disease. In one embodiment, the second sample of DNA is from a human with disease. In one embodiment, the comparing of step (e) comprises gel electrophoresis.

In one embodiment, the present invention contemplates a vector comprising a methylated biomarker sequence, said sequence comprising a disease-specific methylated restriction site pattern.

In one embodiment, the present invention contemplates a method comprising cloning a vector comprising a methylated biomarker sequence, said sequence comprising a disease-specific methylated restriction pattern. In one embodiment, the vector is integrated into a host cell genome. In one embodiment, the host cell is selected from the group comprising a prokaryotic cell, a eukaryotic cell, or a human cell.

In one embodiment, the present invention contemplates a method comprising diagnosing a disease by identifying a disease-specific methylated restriction site pattern. In one embodiment, the methylated restriction site pattern reflects changes in the global methylation of the genome. In one embodiment, the methylated restriction site pattern reflects the methylation status of specific genes associated with the disease. In one embodiment, the methylated restriction site pattern changes in response to the disease progression. In one embodiment, the methylated restriction site pattern changes in response to the disease regression. In one embodiment, the methylated restriction site pattern changes in response to a therapeutic treatment known to reduce symptoms of the disease.

In one embodiment, the present invention contemplates a method comprising identifying a patient having susceptibility to a disease by identifying a disease-specific methylated restriction site pattern.

In one embodiment, the present invention contemplates a method comprising predicting the efficacy of a therapeutic treatment by identifying a disease-specific methylated restriction site pattern.

In one embodiment, the present invention contemplates a method comprising diagnosing an individual's nutritional state by identifying a disease-specific methylated restriction site pattern. In one embodiment, the methylated restriction site pattern changes in response to dietary alterations. In one embodiment, the methylated restriction site pattern changes in response to administration of nutrition supplements.

In one embodiment, the present invention contemplates a GM/RESA method, comprising: a) providing; i) isolated genomic DNA, wherein said DNA comprises at least one restriction site, wherein said restriction site comprises a cytosine residue capable of a 5′-methylation; ii) a methylation sensitive restriction enzyme; iii) a methylation insensitive restriction enzyme; iv) a biotinylated nucleotide selected from the group consisting of cytosine, guanidine, thymidine and adenine; and b) contacting said methylation sensitive restriction enzyme with a first aliquot of said genomic DNA to create a first plurality of restriction fragments; c) contacting said methylation insensitive restriction enzyme with a second aliquot of said genomic DNA thereby creating a second plurality of restriction fragments; d) incorporating said biotinylated nucleotide into said first and second plurality of restriction fragments thereby creating a first and second plurality of biotinylated restriction fragments; and e) detecting said incorporated biotin in said restriction fragments under conditions such that a sample methylation index is calculated. In one embodiment, said detecting of said incorporated biotin is performed using a biotin-specific fluorescent marker. In one embodiment, the isolated genomic DNA is obtained from a patient. In one embodiment, the method further comprises step (f) comparing said sample methylation index with a normal methylation index. In one embodiment, the comparison identifies said calculated methylation index as representing a hypomethylation state. In one embodiment, the hypomethylation state identifies said patient is at risk for a disease. In one embodiment, the hypomethylation state identifies said patient as having a disease. In one embodiment, the DNA is isolated from a diseased cell. In one embodiment, the diseased cell includes, but is not limited to, a cancer cell, a lung cell, a prostate cell, a blood cell, or a buccal cell. In one embodiment, the methyl sensitive restriction enzyme is selected from the group including, but not limited to, HpaII, Aci I, Ava I, Fnu4HI, GlaI, Hinp1 I, HpyCh4 IV, Mwo I, Nla IV, ScRF I. In one embodiment, the methylation insensitive restriction enzyme is selected from the group including, but not limited to, MspI. In one embodiment, two sources of DNA are compared using the method described above (e.g., diseased tissue versus normal). In one embodiment, the two sources comprise diseased tissue from smokers and non-diseased tissue from non-smokers. In one embodiment, the two sources comprise diseased tissue from asthmatics and non-diseased tissue from non-asthmatics.

Definitions

The term “CpG Island”, as used herein, refers to any DNA region wherein the calculated CG % composition is over 50% and the calculated ratio of observed and experimental CG is over 0.6 within a set of averaged “nucleic acid windows” having a total minimum length of 200 nucleotides.

The term “statistically designed primer set”, as used herein, is to be contrasted with a random primer set and refers to any primer set that is biased to hybridize within the boundaries of a CpG Island (i.e., for example, a “CpG-Island Specific Primer”). Further, a statistically designed primer set may encompass various motifs including, but not limited to, GC nucleotide repeats or methyl sensitive restriction sites. The sequence length of a statistically designed primer set is not limited within the present invention and may range from approximately twenty (20)-four (4) nucleic acids, preferably between approximately, fifteen (15)-6 nucleic acids, but more preferably between approximately ten (10)-eight (8) nucleic acids.

The term, “nucleic acid window”, as used herein, refers to any nucleic acid sequence having a specific number of nucleic acids. For example, a nucleic acid window comprise approximately between twenty (20)-six (6) nucleic acids, preferably approximately fifteen (15) nucleic acids, more preferably ten (10) nucleic acids, but more preferably approximately eight (8) nucleic acids.

The term “methylation biomarker”, “disease-specific methylated restriction site pattern” or “methylation fingerprint”, as used herein, refers to any sequence of nucleotides, preferably CpG rich, where the 5′ position of any cytosine base becomes methylated. These regions may be found in any nucleotide sequence including, but not limited to, promoters, regulatory elements, enhancers, and gene coding sequences. Changes in any methylation fingerprint may be an indicator of genome instability and may be useful in the diagnosis of disease. For example, changes in a methylation fingerprint may alter the accessibility of the DNA binding proteins to bind to the DNA.

The term “hypomethylation”, as used herein, refers to any cytosine in a CG or CNG di- or tri-nucleotide site that does not contain a 5′ methyl group. Cell types expressing a hypomethylated state may comprise a housekeeping or non-housekeeping function. For example, these cells may include, but are not limited to, normal cells that express tissue-specific or cell-type specific genetic functions, as well as tumorous and/or cancerous cell types.

The term “hypermethylation”, as used herein, refers to any cytosine in a CG or CNG di- or tri-nucleotide site that does contain a 5′ methyl group. Cell types expressing a hypermethylated state may comprise a housekeeping or non-housekeeping function. For example, these cells may include, but are not limited to, normal cells that express tissue-specific or cell-type specific genetic functions, as well as tumorous and/or cancerous cell types.

The term “global methylation”, as used herein, refers to genome-wide methylation events associated with all CG dinucleotides, all restriction enzyme cutting sites for specific methylation sensitive/insensitive enzyme(s), or all priming events with statistically designed primer set(s).

The term “promoter”, as used herein, refers to a sequence of nucleotides that resides on the 5‘end of a gene’s open reading frame. Promoters generally comprise nucleic acid sequences which bind with proteins such as, but not limited to, RNA polymerase and various histones.

The term “methylation specific enzyme”, as used herein, refers to any enzyme that will cut a nucleic acid sequence only at a CpG site comprising a 5′-methyl cytosine. For example, one methylation specific enzyme is BisI.

The term “methylation sensitive enzyme”, as used herein, refers to any enzyme that will not cut a nucleic acid sequence at a CpG site comprising a 5′-methyl cytosine. Examples of enzymes of this type include, but are not limited to, AatII, AciI, AclI, AgeI, AscI, AsiSI, AvaI, BceAI, BmgBI, BsaAI, BsaHI, BsiEI, BsiWI, BsmBI, BspDI, BsrFI, BssHII, BstBI, BstUI, BtgZI, EagI, FauI, FseI, FspI, HaeII, HgaI, HhaI, HinP1I, HpaII, Hpy99I, HpyCH4IV, MluI, Nael, NarI, NgoMIV, NotI, NruI, PaeR7I, PmlI, PvuI, RsrII, SacIl, SalI, SfoI, SgrAI, SmaI and ZraI.

The term “methylation insensitive enzyme”, as used herein, refers to any enzyme that will cut a nucleic acid sequence at a CpG site with or without a 5′-methyl cytosine. In other words, a methylation insensitive enzyme will cleave a methylation restriction site independent of its methylation status. For example, one methylation insensitive enzyme is MspI.

The term “semi-frequent restriction enzyme”, as used herein, refers to any five base pair (5 bp) restriction enzyme (i.e., a restriction enzyme having a molecular footprint of 5 bp) having genomic frequency of at least 1 in 1,000,000 bp, preferably having a genomic frequency of at least 1 in 100,000 bps but more preferably having a genomic frequency of at least 1 in 10,000 bp.

The term “frequent restriction enzyme”, as used herein, refers to any four base pair restriction enzyme (i.e., a restriction enzyme having a molecular footprint of 4 bp) having a genomic frequency of at least 1 in 10, preferably having a genomic frequency of at least 1 in 100 bp, but more preferably having a genomic frequency of at least 1 in 1000 bp.

The term “complement” or “complementary”, as used herein, when referring to any nucleic acid sequence defines an “antisense” (i.e., reverse order) nucleic acid sequence. A complementary sequence will hybridize to a “sense” nucleic acid under stringent or non-stringent conditions.

The term “digestion” or “digest” or “digesting”, as used herein, when referring to any nucleic acid sequence means sequence-specific cleavage by using a specific restriction enzyme. Exemplary restriction enzymes are commercially available with reaction conditions, cofactors and other requirements for use provided as instructions. For example, a 1 μg of plasmid or DNA fragment may be digested with about 2 units of a restriction enzyme in about 20 μl of an appropriate reaction buffer. Alternatively, 5 to 50 μg of DNA may be digested with 20 to 250 units of restriction enzyme in proportionately larger volumes. Incubation times of about 1 hour at 37° C. may be used, but 12 hour incubations (i.e., for example, overnight) may also be employed.

The term “isolated”, as used herein, refers to any alteration or removal of a substance or compound from its original, natural, environment. For example, a polynucleotide or a polypeptide naturally present in a living animal's cells in its natural state may be “isolated” by separation from the cellular structure.

The term “fusion protein”, as used herein, refers to any expressed protein encoded by one or more polynucleotides. For example, the encoding polynucleotide may result from the joining of an isolated polynucleotide and a synthetic polynucleotide. Further, one or more polynucleotides may comprise a mutation when compared to the template (i.e., wild type) polynucleotide sequence.

The term “vector” as used herein, refers to any polynucleotide sequence, including, but not limited to, isolated polynucleotides that are alone or joined to other polynucleotides capable of introduction into host cells. For example, such host cells may be an in vitro cell culture or an in vivo tissue and/or organ. The term “vector” further may refer to any nucleotide sequence comprising a gene of interest operably linked to a promoter complex. In some embodiment, such a vector may be stably, or transiently, integrated into the genome of a host cell. During such integration, the gene of interest may be expressed wherein the vector transcripts are translated into protein by the host cell protein translation machinery.

The term “ligation”, as used herein, refers to any process of forming phosphodiester bonds between two or more polynucleotides, such as those comprising double stranded DNAs. Techniques and protocols for ligation may be found in standard laboratory manuals and references. Sambrook et al., In: Molecular Cloning. A Laboratory Manual 2nd Ed.; Cold Spring Harbor Laboratory Press, Cold Spring Harbor, N.Y. (1989) and Maniatis et al., pg. 146.

The term “nucleic acid” or “polynucleotide”, as used herein, refers to any purine- and pyrimidine-containing polymer of any length, either as polyribonucleotides, polydeoxyribonucleotides, or mixed (i.e., polyribo-polydeoxyribo) nucleotides. Such polymers may include, but are not limited to, single-and double-stranded molecules, i.e., DNA-DNA, DNA-RNA and RNA-RNA hybrids, as well as “protein nucleic acids” (PNA) formed by conjugating nucleic acid bases to an amino acid backbone. Any nucleic acid containing a modified bases.

The term “oligonucleotide” or “oligonucleotides”, as used herein, refer to relatively short (e.g., 5 to 100 bases) polynucleotides as defined above. Oligonucleotides are often synthesized by chemical methods, such as those implemented on automated oligonucleotide synthesizers. However, oligonucleotides can be made by a variety of other methods, including, but not limited to, in vitro recombinant DNA-mediated techniques and/or by expression of DNAs in transfected cells and organisms.

The term “plasmid”, as used herein, refers to any extrachromosomal ring of DNA that replicates autonomously. Plasmids are useful for cell transfection, wherein a gene of interest is incorporated into the plasmid. Once the plasmid is in the host cytoplasm the gene of interest is expressed using the plasmid's transcription control elements. Generally, plasmids are designated a lower case “p” preceded and/or followed by capital letters and/or numbers indicating the plasmid source. Plasmids are either commercially available or can be constructed using standard genetic engineering protocols.

The term “probe”, as used herein, refers to any nucleic acid or oligonucleotide that forms a hybrid structure with a sequence of interest in a target gene region due to complementarily of at least one sequence in the probe with a sequence in the target region.

The term “substantially homologous” or “substantially similar” as used herein, when referring to nucleic acid sequences, means that upon optimal alignment of two nucleic acid sequences the nucleotide sequence identity is at least approximately 60%, preferably at least approximately 70%, more preferably at least approximately 80%, even more preferably at least approximately 90%, and most preferably at least approximately 95-98%.

The term “selective hybridization” as used herein, refers to hybridization having at least about 55% homology over a stretch of at least about nine or more nucleotides, preferably at least about 65%, more preferably at least about 75%, and most preferably at least about 90%. The length of homology comparison, as described, may be over longer stretches, and in certain embodiments will often be over a stretch of at least about 14 nucleotides, usually at least about 20 nucleotides, more usually at least about 24 nucleotides, typically at least about 28 nucleotides, more typically at least about 32 nucleotides, and preferably at least about 36 or more nucleotides. It should be understood that the concepts of “substantial homology/similarity” and “selective hybridization” are for all practical purposes, synonymous.

The term “variant” or “variants” as used herein, refer to polynucleotides or polypeptides that respectively differ in nucleic acid or amino acid composition and/or sequence relative to a reference polynucleotide or polypeptide. Variants may have, but not necessarily, properties of “selective hybridization” relative to the reference polynucleotide or polypeptide.

The term “cloning” as used herein, refers to any in vitro recombination technique that inserts a gene of interest with or without any other nucleic acid sequence into a vector and/or plasmid. For example, cloning may involve methods including, but not limited to, nucleic acid fragment generation, joining or ligating nucleic acid fragments to vectors and/or plasmids, introducing and/or transfecting a host cell with the joined and/or ligated vector/plasmid, and selecting one or more clones expressing the nucleic acid fragment from amongst all the recipient host cells.

The term “host cell” as used herein, refers to any biological cell (i.e., for example, animal, mammalian, plant, bacterial, insect, etc) that is capable of transfection by a vector and/or plasmid. A host cell may include, but is not limited to, prokaryotes and eukaryotes.

### BRIEF DESCRIPTION OF THE FIGURES

FIG. 1 presents one embodiment of a Global Methylation Restriction Enzyme Sensitive Assay (GM-RESA) methodology detecting changes in global nucleic acid methylation.

FIG. 2 presents exemplary data resulting from a GM-RESA on various lung cell lines. Panel A: DNA digested with HpaI normalized against MspI digestion. Panel B: DNA digested with BssHII normalized against MspI digestion. Lane 1: Normal lung (NL20). Lane 2: Stage I lung cancer (NCI-H1703). Lane 3: Stage II lung cancer (NCI-H522). Lane 4: Stage IIIa lung cancer (NCI-H1993). Lane 5: Stage IIIb lung cancer (NCI-H1944). Lane 6: Stage IV lung cancer (NCI-H1755). Lane 7: Small cell lung cancer (NCI-H2126). Lane 8: Non-small cell lung cancer (NCI-H69).

FIG. 3 presents one embodiment to detect nucleic acid methylated restriction sites using a methylation sensitive restriction enzyme digest assayed by either Global Methylation/CpP-Specific Primers (GMSP) or Methylation Fingerprinting/CpP-Specific Primers (MFSP). CG: Unmethylated restriction site. C*G: Methylated restriction site. Arrow: Methylation sensitive restriction enzyme cleavage site. FP: Forward CpG primer. RP: Reverse CpG primer.

FIG. 4 presents an illustration comparing the sequence homology of random nucleic acid primers versus CpP-specific primers to CpG Island nucleic acid sequences. Panel A: Logarithmic correlation analysis using random 10mer primer pairs. Panel B: Logarithmic correlation analysis using CpG-specific primer pairs. Panel C: Cumulative distribution frequency of the number of predicted PCR fragments. X Axis: Number of predicted PCR products shorter than 2000 bp. Y Axis: Percentage of primer pairs that amplify equal or less than a specific predicted number of fragments. Dashed Line: Random primer pairs. Solid Line: CpG-specific primer pairs.

FIG. 5 presents one embodiment of a Quantitation of Methylation in Specific Regions of the Genome (MSRquant) methodology detecting nucleic acid methylation.

FIG. 6 presents one embodiment of a Methylation Sensitive Amplification System (MESAS) to identify novel DNA methylation biomarkers.

FIG. 7 presents exemplary data using MESAS that identifies nucleic acid methylation biomarkers in asthmatic lung tissue. Arrows: Electrophoretic gel nucleic acid bands that are different (i.e., changes is either intensity and/or appearance) between asthmatic lung tissue and normal lung tissue. Lane 1: Patient A asthmatic lung tissue. Lane 2: Patient B asthmatic lung tissue. Lane 3: Patient C asthmatic lung tissue. Lane 4: Patient D normal lung tissue. Lane 5: Patient E normal lung tissue. Lane 6: Patient F normal prostate tissue. Lane 7: 100 bp ladder nucleic acid standards.

FIG. 8 presents exemplary data using MFSP to provide nucleic acid methylation fingerprinting. Asterisks: Methylated PCR products. Arrows: Unmethylated PCR products. Lane 1: Methylation sensitive restriction enzyme digest (Hpall) Lane 2: Methylation sensitive & insensitive restriction enzyme digest (Hpall+Msp1). Lane 3: Methylation insensitive restriction enzyme digest (Msp1). Lane 4: Methylation sensitive & insensitive restriction enzyme digest (Hpall+Msp1).

FIG. 9 presents one embodiment of a computer program designed to select a statistically designed primer set having a bias to hybridize within the boundaries of a CpG Island.

FIG. 10 presents and exemplary outline of an effect of environmental modifiers on the methylation status of the genome. Hypomethylation may reflect an early stage event in the progression of disease. Measurement of global DNA methylation at these early stages might identify individuals that are in a pre-neoplasia stage as opposed to measuring region specific methylation, where markers need to be identified that are associated with the disease and show changes that occur when the cell in an advanced stage of neoplasia.

FIG. 11 presents an illustrative drawing showing a high level of methylation (i.e., for example, 70%) that normally occurs throughout the genome. The methylation occurs at the cytosine in a CpG dyad, which may serve as sentinels of genome integrity.

FIG. 12 presents one embodiment of an overview of one methodology of GM-RESA to measure changes in global DNA methylation.

FIG. 13 presents one embodiment of an overview of one concept to measure global DNA methylation in a 96 well microtiter plate.

FIG. 14 presents exemplary data showing an effect on luminescence by varying the amount of biotinylated dGTP and dCTP using a fixed amount of genomic DNA.

FIG. 15 provides exemparly data comparing streptavidin with neutravidin on signal to background noise. 15A: Graphs showing the effect on luminescence using streptavidin and neutravidin with varying amounts of genomic DNA; 15B: The linear curves of the two avidins with varying amounts of genomic DNA.

FIG. 16 provides exemparly data showing an effect of varying the end-fill conditions on luminescence and signal to noise background using Klenow. D=HpaII digested DNA; UD=undigested DNA; Star=optimal conditions.

FIG. 17 provides exemplary data evaluating optimal conditions for an end-fill reaction using Sequenase. 17A: Graph showing the effect of varying the end-fill conditions on luminescence and the signal to noise background using a fixed amount of Sequenase. D=HpaII digested DNA; UD=undigested DNA; and Star=optimal conditions; and 17B: Graph showing effects of titrating Sequenase on luminescence using the optimal conditions identified in 17A.

FIG. 18 provides exemplary data showing a linear relationship between luminescence and varying amounts of genomic DNA digested with MspI.

FIG. 19 provides exemplary data showing successful normalization between varying amounts of genomic DNA by using a Methylation Index.

FIG. 20 provides exemplary data showing GM-RESA luminescence linearity using various concentrations of genomic DNA digested with MspI (20A) and HpaII (20B).

FIG. 21 provides exemplary data showing a correlation of GM-RESA with HPCE. The X-axis represents the triplicate results from GM-RESA using DNA from 4 cell lines digested with HpaII and normalized with MspI. The Y-axis represents the HPCE results published by Paz et al, 2003 using the same 4 cell lines that were used in the GM-RESA. Line=linear regression fit.

FIG. 22 provides exemparly data assessing the analytical sensitivity of GM-RESA using Lambda DNA. 22A: Luminescence using various amounts of lambda DNA; 22B: Linearity of GM-RESA using mixtures of methylated and unmethylated Lambda DNA between 100% to 10% (in 10% increments); 22C: Linearity of GM-RESA using mixtures of methylated and unmethylated Lambda DNA between 100% to 45% (in increments of 5%).

FIG. 23 provides exemplary data measuring global DNA methylation in various lung and prostate cancer cell stages. Dotted line: Methylation index in a normal cell line. Note: A methylation index above the dotted line indicates a state of hypomethylation.

FIG. 24 provides exemplary data to identify an optimal restriction enzyme to serve as a quantitative biomarker to measure global DNA methylation. Seventeen (17) methyl sensitive enzymes were digested with DNA from normal and tumor lung cell lines followed by a GM-RESA protocol.

FIG. 25 provides exemplary data that quantitatively assesses potential biomarkers for global DNA methylation. X Axis: Mixtures of normal lung:tumor cell DNA between 0:100-100:0 in 10% increments. Enzymes in 25A-25F showed a linear increase in hypomethylation starting between 5:95 (5%) to 10:90 (10%) up to 100:0 (100%) tumor/normal ratio. Enzymes in 25F & 25G showed a linear increase in hypomethylation only up to 50:50 (50%) tumor/normal ratio.

FIG. 26 provides exemplary data showing a methyl index for normal non-disease (open bar), normal disease (crosshatched bar) and the paired tumor (stippled bar) for each indicated methyl sensitive enzyme.

FIG. 27 provides exemplary data using GM-RESA to obtain DNA Methyl Indicies from buccal cells taken from smokers and non-smokers. The graph shows that smokers have a higher methylation index than the non-smokers when using the HpaII methyl sensitive enzyme suggesting that hypomethylation is occurring in the genome of the smokers. Dotted Line: Average normal DNA Methylation Index, wherein a higher index indicates a state of hypomethylation.

FIG. 28 provides exemplary data showing methyl indicies for normal samples (open bar) and asthma lung DNA samples (crosshatched bar) for each indicated methyl sensitive enzyme.

FIG. 29 provides exemplary data showing a linear curve of genomic DNA digested with MspI. In this embodiment, linearity is observed from 00 ng to 12.5 ng DNA.

FIG. 30 provides exemplary data showing successful normalization of methylation indicies using a 384 well microtiter plate at varying amounts of genomic DNA.

FIG. 31 provides exemplary data titrating Sequenase in an end-fill reaction with biotinylated dCTP and dGTP performed in a 384 well microtiter plate using 25 ng genomic DNA digested with HpaII and normalized against MspI.

FIG. 32 provides exemplary data assessing the analytical sensitivity of GM-RESA in a 384 well microtiter plate using Lambda DNA. 32A: Linearity of GM-RESA using mixtures of methylated and unmethylated Lambda DNA from 100%-1-% in increments of 10%; 32B: Linearity of GM-RESA using mixtures of methylated and unmethylated Lambda DNA from 100% to 45% in increments of 5%.

### DETAILED DESCRIPTION OF THE INVENTION

The present invention relates to methods of detecting changes in DNA methylation patterns. In one embodiment, DNA methylation patterns are detected by ligating a DNA fragment before digestion with a methylation insensitive restriction enzyme and/or a methylation sensitive restriction enzyme. In another embodiment, DNA methylation biomarkers are identified using primer pairs selective for a CpG Island. Such changes in DNA methylation patterns may provide disease diagnosis, prognosis, and potential therapeutics as well as determining general health.

In some embodiments, the invention contemplates methodologies that measure the changes in nucleic acid (i.e., for example, DNA) methylation both at the areas around genes in particular promoters of genes as well as throughout the genome. Other embodiments of the invention contemplate methodologies to detect DNA methylation that relates to genome instability that leads to a disease state(s) or a change in general health. For example, discovery of novel DNA methylation biomarkers that are associated with disease or changes in DNA methylation in response to dietary changes and/or nutritional supplement use.

I. Genomic Functions of Nucleic Acid Methylation

Nucleic acid methylation is suspected to play a role in nucleic acid transcription, and consequently may have some overall impact on in vivo protein production. Some embodiments of the present invention have not only confirmed those suspicions but have identified specific nucleic acid sequences that are altered in specific disease states.

Within the context of human disease, phenotypic variation has been attributed to the interaction of genetic predispositions such as an at-risk or protective haplotype with the influences of the environment. The influence of the environmental effects is considered a major factor for many common diseases because the concordance rates among monozygotic twins do not approach 100%. In fact, disease rates vary widely with geography and culture. However, the environment and genetic predisposition do not account for the discrepancy in concordance rates observed in monozygotic and dizygotic twins, which in one study for type II diabetes was found to be 63% and 43% and in another study on bipolar disorder was found to 67% and 20%, respectively (Bjornsson et al., 2004). The role of epigenetics as a potential third determinant that influences disease is being widely considered as the missing component in explaining the idiosyncrasies of complex disorders.

A. Epigenetics

Epigenetics may be defined as a stable and potentially heritable form of cellular information that influences gene expression but does not involve a change in the DNA sequence (i.e., is non-mutagenic). This cellular information is in the form of covalent modifications applied to the histones and nucleic acids. In histone proteins, one form of this cellular information may be exemplified by post-translation modifications including, but not limited to, phosphorylation, acetylation, methylation, poly-ADP ribosylation and ubiquination. In nucleic acids, one form of this cellular information may be exemplified by nucleic acids comprising 5′ methylated cytosines.

There are at least three inter-related types of epigenetic inheritance: i) nucleic acid methylation; ii) genomic imprinting; and iii) histone protein modification. Nucleic acid methylation occurs at CpG dinucleotides and plays a role in the regulation of gene expression as well as the silencing of repeat elements in the genome. Genomic imprinting comprises parent-of-origin-specific allele silencing mediated by differentially methylated regions within or near imprinted genes that may be normally reprogrammed in the germline. Histone modifications include, but are not limited to, methylation, acetylation, and phosphorylation are involved in transcriptional regulation wherein many histone modifications are stably maintained during cell division. Enzymes that mediate histone modifications are often associated within the same genomic complexes as those that regulate nucleic acid methylation (i.e., for example, CpG Islands).

Although it is not necessary to understand the mechanism of an invention, it is believed that histone protein modifications and their positioning on nucleic acids may restructure the genome into either open or condensed chromatin. An open or condensed chromatin structure is further believed to regulate the accessibility of the DNA for transcription, methylation, recombination, replication and repair. These histone positioning and modifications may be referred to as “epigenetic memory” and/or “genomic imprinting” and constitutes the stable heritable form of epigenetics (i.e., thereby generating an “epigenotype”).

However, a mutation may alter the covalent modifications on the histones and DNA, wherein the net effect may be a remodeling of the architecture of the chromatin within the three-dimensional space of the nucleus, possibly causing a perturbation in the expression profile of genes in that cell. This can result in an array of diseases such as cancers and multi-system developmental disorders such as asthma, type II diabetes, bipolar disorder, multiple sclerosis and heart disease.

Epigenetic regulation at the nucleic acid level has been reported to be mediated by covalent modifications (i.e., for example, 5′ cytosine methylation). For example, a methyl group may be added to a cytosine carbon-5 position that is part of a symmetrical group of CpG dinucleotides. Many 5-methylcytosines have been found in retrotransposons, endogenous retroviruses and repetitive sequences, which may have evolved as a host defense mechanism to prevent the mobilization of these elements and reduce the occurrence of chromosomal rearrangements (Jiang et al, 2004).

Unmethylated CpG dinucleotides may be found in short CpG-rich sequences, commonly referred to as CpG Islands. CpG Islands have been observed to cluster in or around promoter regions of genes. One report indicates that over 40% of protein encoding genes have at least one CpG Island that is found within the vicinity of their promoters (Yu et al, 2004).

CpG Island methylation may reduce the competency of expression. Further, if hypermethylation occurs, it is possible that the gene might become completely switched off. Consequently, epigenetic regulation may be able to respond to environmental influences by gradually changing a gene's methylation status. The removal of such environmental influences would then be expected to reverse a gene's methylation status and appropriately adjust the expression profile of that gene in the cell. Thus, epigenetic regulation may be elastic in nature and able to dynamically respond to fluctuations in the environment. It is not believed that a nucleic acid genotype is capable of such a control system.

Since epigenetic regulation may exert an influence on genotypic expression, this capability has the potential to activate an at-risk gene haplotype or a protective gene haplotype. Consequently, epigenetic modification of genotypic expression may help to explain the broad spectrum of phenotypes observed in patients affected with complex diseases (i.e., for example, cancer).

B. Genomic Stability

The genome is susceptible to adverse impacts following exposure to “biological attack” from agents including, but not limited to, oxidant stress, carcinogens, and other deleterious environmental factors. Genome damage has been reported to have an adverse impact on all stages of life including, but not limited to, infertility, fetal development, and accelerated aging, as well as cancer and other degenerative diseases. (Fenech M. 2005. Mutagenesis 20:255-269).

Nucleic acid methylation has been suggested to be one example of a “host defense system” that guards the genome from these adverse events and may be responsible for “optimal genome maintenance”. (Fenech M. 2005. Mutagenesis 20:255-269; McCabe D C and Caudill M A. 2005. Nutrition Reviews 63:183-195). Nucleic acid methylation might maintain genome stability by directly stabilizing chromosomes and chromatin compartmentalization, silencing parasitic and viral DNA expression (i.e. LINEs and SINEs), maintaining genomic imprinting and X-chromosomal inactivation, and suppressing certain genes for tissue-specific expression. (Egger G et al. 2004. Nature 429:457-463; Esteller M (ed.) 2004. DNA Methylation Approaches, Methods, and Applications. pp 27-52).

Nucleic acid methylation patterns established during an organism's development are able to stably and heritably silence gene expression. Genomic stability may partly be maintained by nucleic acid methylation through compartmentalization, transcriptionally active euchromatin, and transcriptionally active heterochromatin. Nucleic acids maintained as chromatin is suspected to protect genome integrity. Genomic instability, on the other hand, has been reported due to germ line or somatic mutations as well as epigenetic mutations. (Lengauer C et al. 1998. Nature 396:643-649).

Three types of genomic instability that might be influenced by nucleic acid methylation are microsatellite instability, chromosomal instability and chromosomal translocation. Microsatellite instability can occur because of point mutations in genes of the mismatch repair system or hypermethylation in the CpG Island of mismatch repairs genes. An increased rate in genomic mutations, especially in microsatellite repeats, has been reported. (Eshleman J R. and Markowitz S D. 1995. Curr Opin Oncol 7:83-89).

The gain or loss of whole chromosomes (aneuploidy) may be observed during chromosomal instability. Nucleic acid hypomethylation patterns has been associated with such instability. Loss of genomic integrity has been attributed to hypomethylation of repetitive elements, which can lead to inappropriate recombination resulting in defects in cell cycle monitoring check point genes as well as genes involved chromosome condensation, kinetochore structure and function, and centrosome and kinetochore formation. (Lengauer C et al. 1998. Nature 396:643-649). Chromosomal breakage and translocations such as the ones observed in the rare recessive genetic disorder ICF (immunodeficiency, centromeric region instability, facial anomalies) are suggested to be due to mutations in the methyltransferase gene DNMT3b. (Xu G L et al. 1999. Nature 402: 187-191). Chromosomal translocations caused by an inactive methyltransferase may result from a failure to methylate the juxtacentromeric regions of chromosomes 1, 9 and 16. This may result in the formation of abnormal, multiradial chromosomes having 3 to 12 arms joined at the pericentromeric region. Also, the failure of DNMT3b to methylate pericentromeric regions may lead to chromatin decondensation thereby making these areas more susceptible to increased recombination. Further, hypomethylation-induced translocations have been observed in multiple myeloma (Sawyer J R et al. 1998. Blood 91:1732-1741). In effect, nucleic acid methylation serves as a stabilizing agent in genomic structures comprising large amounts of repetitive elements by preventing recombination across these regions. (Eden A et al. 2003. Science 300:455).

An alternative approach for determining nucleic acid stability and/or integrity involves a micronuclei (MN) assay. MN are believed to originate from chromosome fragments or whole chromosomes that lag behind at anaphase during nuclear division. The MN index can be used in vivo and/or ex vivo in rodent and/or human cells to measure the genetic toxicology of chemicals and radiation. Kassie F et al. 2001. Int J Cancer 92:329-332); and (Moore L E et al. 1996. Environ Mol Mutagen 27:176-184), respectively. The MN index can be measured in erythrocytes, buccal cells or lymphocytes with little difficulty to ascertain the extent of genome damage. (Fenech M. 2005. Mutagenesis 20:255-269). The buccal MN assay has been used to study: i) genome damage in Bloom's syndrome (Honma M et al. 2002. Mutat Res 520:15-24); and ii) dietary-induced DNA damage (Stich H F et al. 1984. Int J Cancer 34:745-750, Piyathilake C J et al. 1995. Cancer Epi Biom Prev 4: 751-758, Titenko-Holland N et al. 1998. Mutat Res 417:101-114 and Majer B J et al. 2001. Mutat Res 489:147-172). One improvement to the MN assay involves the cytokinesis-block MN(CBMN) assay which measures the MN index in human lymphocytes that have only undergone one cell division. (Fenech M and Morley A A. 1999. Mutat Res 161:193-1980. Specifically, CBMN uses cytochalasin-B to arrest cytokinesis thereby giving the once-divided cells a binucleated appearance. Others have evolved this assay into a comprehensive method for the measurement of chromosome breakage, chromosome loss, non-disjunction, gene amplification, necrosis, apoptosis and cytostasis. (Fenech M. 2005. Mutatagenesis 20:255-269).

However, the MN and the CBMN assays are not suitable methodologies that will convert easily to automation procedures. A biochemical assay that can measure genome stability in a more sensitive and high-throughput manner is still lacking and highly needed. In one embodiment, the present invention contemplates methylation detection methods that address an unmet need to measure in a non-invasive approach the genomic stability and determine the general well being of an individual.

C. Nutrition

Dietary nutrition has been implicated in many pathways involved in apoptosis, cell cycle control, differentiation, inflammation, angiogenesis, DNA repair and carcinogen metabolism. (Davis C D and Uthus E O. 2004. Exp Biol Med. 229:988-95). These cellular pathways are believed regulated by nucleic acid methylation and other epigenetic events. Consequently, nucleic acid methylation may constitute a mechanism by which dietary components can modulate genome stability and gene regulation.

Nutritional modulation of nucleic acid methylation may involve single carbon metabolic pathways. These nutrients may include, but are not limited to, vitamin B12, vitamin B6, folate, methionine and choline. Some reports suggest that the nutrients influence the supply of methyl groups and, therefore, affect the biochemical pathways of methylation processes. (McCabe D C and Caudill M A. 2005. Nutrition Reviews 63:183-195, Davis C D and Uthus E O. 2004. Exp Biol Med. 229:988-95). Further, other studies suggest that folate intake/status modulates nucleic acid methylation in humans. (McCabe D C and Caudill M A. 2005. Nutrition Reviews 63:183-195). For example, one depletion-repletion study clearly show >100% increase in nucleic acid hypomethylation after 9 weeks on low folate diet and a subsequent increase in nucleic acid methylation after a further 3 weeks on a high folate diet. (Jacob R A et al. 1998. J. Nutr. 128:1204-1212).

Other nutrients have also been shown to affect nucleic acid methylation. These nutrients include, but are not limited to, alcohol, arsenic, cadmium, coumestrol, equol, genistein, nickel, selenium, tea polyphenols, vitamin A, and zinc. (Davis and Uthus 2004). Many of these nutrients including, but not limited to, zinc, selenium, genistein, tea polyphenols, and vitamin A have also been associated with cancer susceptibility. Some believe that either deficiencies or excess of these nutrients could cause abnormal methylation profiles. (Davis C D and Uthus E O. 2004. Exp Biol Med. 229:988-95). Others suggest that nucleic acid methylation might be useful as a “biodosimeter” that may be able to determine the optimum amounts of certain dietary components needed to maintain the genomic health. (Fenech M. 2005. Mutagenesis 20:255-269, Davis C D and Uthus E O. 2004. Exp Biol Med. 229:988-95).

D. Disease

Data supporting a link between nucleic acid instability (supra) and disease is becoming increasingly stronger. For instance, epigenetic nucleic acid regulation plays a part in the physiologic and pathologic events associated with aging and cancer.

Presently, methylation of the 5′ cytosine in CpG dinucleotides is believed to be the only reported naturally-occurring nucleic acid modification. In adult human cells, reports indicate an approximate 70% methylation rate of CpG dinucleotides. Nucleic acid methylation has been implicated in chromatin structure, chromosomal stability, silencing repetitive sequences as well as a defense mechanism against the deleterious effects of integrated foreign DNA.

Genomic instability is a fundamental characteristic of disease initiation and progression, an observation that has been made in cancer. Some preinvasive lesions are committed to develop into invasive cancers. (Venmans B J et al. 2000. Chest 117:1572-1576 and Bota S et al. 2001. Am J Respir Crit. Care Med 164:1688-1693). Several mechanisms may predispose a lesion to develop into cancer involving molecular abnormalities including, but not limited to, somatic mutations, chromosomal aberrations and mutagens.

1. Hypo-Hypermethylation

To maintain the stability of the genome, two major alterations in nucleic acid methylation have been observed: hypomethylation and hypermethylation. In some diseases, one or the other of these methylation states may be prevalent depending upon the gene locus. In neoplasia, for example, an overall genomic hypomethylation is present, in conjunction with a hypermethylation of promoter-associated CpG Islands. The hypomethylated state is believed to silence some tumor-suppressive gene activity. Consequently, one embodiment of the present invention contemplates that both hypomethylation and hypermethylation represent epigenetic dysregulation that is responsible for the development, expression and maintenance of cancer and/or tumors.

Genome-wide (global) loss of 5′-methyl cytosine is one of the earliest molecular abnormalities described in human neoplasia. Global demethylation has been shown to occur mostly outside of promoters in CpG-depleted areas as well as in repetitive elements and pericentric bulk DNA. Hypomethylation has mechanistic implications and can play a role in neoplasia through the activation and over-expression of growth promoting genes (i.e., for example, HRAS). Also, hypomethylation has been shown to play a role in the induction of chromosomal instability leading to neoplasia. The pericentromeric satellite regions are vulnerable to hypomethylation causing unbalanced chromosomal translocations, which have been observed in ovarian and breast carcinomas. Hypomethylation of L1 retrotransposons has been shown to be correlated with chromosomal instability colorectal cancer cell lines.

Hypermethylation causation has not been fully determined but appears to involve a combination of: i) hypersensitivity to methylation in some CpG Islands; ii) a defect in the de novo methylation process; and iii) a selection for cells having inactivated growth-suppressor genes. This deadly combination is believed to be a major contributor to neoplasia.

Sixty percent of expressed genes have 5′ regions and upstream promoters that are located in 0.5 to 3.0 Kb nucleic acid stretches unusually rich in CpG dinucleotides. These clusters of CpG sites are known as CpG Islands and are generally free of DNA methylation. De novo nucleic acid methylation of gene promoters has been a consistent abnormality in human neoplasia. Promoter methylation has been linked to the inactivation of tumor-suppressor genes (i.e., for example, RB1, P16, BRCA1 and VHL), DNA repair genes (i.e., for example, hMLH1 and MGMT), angiogenesis inhibitors (i.e., for example, THBS1), and growth regulators (i.e., for example, ER and PGR).

2. Imprinting

The role of imprinted genes in human development was first observed in two neurodevelopmental disorders, Prader-Willi syndrome (PWS) and Angelman syndrome (AS). These two disorders were found to be due to uniparental chromosomal disomies of the long arm of chromosome 15. This feature was found to be maternal in PWS and paternal in AS.

In Wilms tumors, a parent-of-origin bias in Loss of Heterozygosity (LOH) was observed on chromosome 11p15 alleles that may be mediated by a Loss of Imprinting (LOI) and a pathological bi-allelic expression of IGF₂. Further, an epigenetic alteration was demonstrated by Histone 19 (H19) hypermethylation found in both the tumor and in non-neoplastic kidney parenchyma. Hypermethylation effects are the earliest observed changes during Wilms tumor development while altered methylation due to imprinting is observed in later stages of disease progression. The latter is due to classical genetic changes.

3. Histone Modifications

Modification of histones by methylation, acetylation and phosphorylation has been shown to play a role in maintaining the stability of the genome by silencing genes. However, silencing inappropriate genes by histone modification can have adverse effects. For example, the methylation of lysine 9 (Lys⁹) of histone H3 has been shown to silence the CDKN2A tumor suppressor gene in some cancer cells.

The machinery that is responsible for the modification of chromatin and nucleic acids work in a co-operative manner to silence genes in normal and malignant cells. There is evidence to indicate that there is cross-talk between histone and nucleic acid methylation machinery that makes this multi-protein complex a likely target for environmental carcinogens. (Feinberg A P. 2004. Seminars in Cancer Biology 14:427-432).

4. Environmental Toxins

Environmental toxins (i.e., for example, mutagens) can cause molecular damage through the formation of nucleic acid adducts. For example, cigarette smoking is a well documented mutagen that has been shown to form nucleic acid adducts that escape normal adduct repair mechanisms. These mutations are believed to result in heritable alterations in the nucleic acid sequence. (Massion P P and Carbone D P. 2003. Respiratory Research 4:120). Benzo(a)pyrene is also considered a nucleic acid-damaging carcinogen and is one of a multitude of polycyclic aromatic hydrocarbons commonly found in tobacco smoke and/or the ambient environment (i.e., for example, industrial pollution, automobile exhaust, or second-hand tobacco smoke). The activated form of this carcinogen can cause nucleic acid adducts, which can lead to point mutations as well as single strand chromatid breaks that have been observed to be more frequent in lung cancers. (Wei Q et al. 1996. Cancer Res 56:3975-3979). Mutagen studies on chromosome breakages revealed that chromosome 4 breaks were significantly associated with a positive family history of cancer in first-degree relatives. (Zhu Y et al. 2002. Cancer Genet Cytogenet 136:73-77).

In one embodiment, the present invention contemplates a method of identifying genomic instability by detecting changes in a nucleic acid methylation pattern, wherein the methylation pattern serves a diagnostic monitor for the development of lung cancer.

II. Methods of Detecting Global DNA Methylation

Nucleic acid methylation biomarker detection methodologies include, but are not limited to: i) biomarker discovery methods; ii) biomarker validation methods; and iii) biomarker screening methods. Restriction site sensitive enzymes (i.e., for example, methylation sensitive restriction enzymes) coupled with a polymerase chain reaction (PCR) step have been utilized to generate several methylation detection approaches; i) “Methylation Sensitive Arbitrary Primed PCR” (Gonzalgo M L et al. 1997. Cancer Res 57:594-599); ii) “Methylated CpG Island Amplification” (Toyota M et al., “Identification of differentially methylated sequences in colorectal cancer by methylated CpG Island amplification” Cancer Res 59:2307-2312 (1999)); iii) two dimensional gel separation termed “Restriction Landmark Genome Screening” (Hayashizaki Y et al. 1993. Electrophoresis 14:251-258); iv) a hybridization procedure termed “Differential Methylation Hybridization” (Huang T H et al. 1999. Hum Mol Genet); v) “Expressed CpG Island Sequence Tag” (Shi H et al. 2002. Cancer Res 62:3214-3220); and vi) “Methylation Amplification DNA Chip” (Hatada I et al. 2002. J Hum Genet. 47:448-451).

A biomarker validation method may be applied once nucleic acid methylation biomarkers have been discovered in order to identify the most promising candidates. For example, methylation can be monitored using a methylation-sensitive restriction enzymes or through the use of chemical modifications of the DNA. In the later case, sodium bisulphate is mixed with nucleic acids and converts non-methylated cytosines to uracil. Methylation sites may then be determined by: i) direct nucleic acid sequencing (Fommer M et al. 1992. Proc Natl Acad Sci USA 89:1827-1831); ii) oligonucleotide microarray hybridization (Gitan R S et al. 2002. Genome Res 12:158-164, Adorjan P et al. 2002. Nucleic Acids Res 30:e21 and Balog R P at al. 2002. Anal Biochem 309:301-310); and various forms of polymerase chain reactions such as: i) “Combined Restriction Analysis” (Xiong Z and Laird P W. 1997. Nucleic Acids Res 25:2532-2534); ii) “Methylation Sensitive PCR” (Herman J G et al. 1996. Proc Natl Acad Sci USA 93:9821-9826), iii) MethyLight (Eads C A et al. 2000. Nucleic Acids Res 28:E32); and iv) “HeavyMethyl” (Cottrell S E and Laird P W. 2003. NY Acad Sci 983:120-123).

Once the DNA methylation biomarkers have been validated, their use in a clinical setting requires methylation methodologies that are relatively simple, reproducible and automatable. The currently practiced PCR-based methods discussed above are highly sensitive but tend to be rather convoluted and cumbersome to perform. Thus, they are not desirable candidates to transition an assay method from a research environment to a clinical setting. In one embodiment, the present invention contemplates nucleic acid methylation detection methods that can easily be performed in a clinical environment and fulfill the unmet needs for diagnosis, prognosis and the monitoring of the efficacy of a therapeutic treatment.

A sample of genomic nucleic acids (i.e., for example, DNA) may be isolated from a biological cell. Biological cells may be derived from a cell line (i.e., in vitro) or derived from a living tissue or organ including, but not limited to, buccal, lung, prostate, kidney, muscle, intestinal, stomach, brain, or peripheral nerves (i.e., for example, in vivo). Further biological samples may be derived from biological fluids, excretions, or secretions including, but not limited to, blood, stool, spinal fluid, saliva, urine, and other bodily fluids.

Commercially available nucleic acid isolation kits can be used to separate the nucleic acids from the other cellular material. Subsequently, the quantity and quality of the nucleic acid material is determined, also by commercially available kits and methods. In general, the methodologies described in this invention are applicable to but not limited to DNA.

A. Global Methylation Restriction Enzyme Sensitive Assay (GM-RESA)

1. Introduction

In the human genome, it is believed that approximately 70% of CpG dinucleotides are methylated in adult cells. Although it is not necessary to understand the mechanism of an invention, it is believed that to maintain genome stability a balanced methylation status is involved: hypomethylation versus hypermethylation. For example, a decrease in methylation (i.e., for example, hypomethylation) is observed to occur mostly outside of promoters in CpG depleted areas as well as in repetitive elements and pericentric bulk DNA. Further, an increase in methylation (i.e., for example, hypermethylation) is observed to occur within CpG islands that are present in ˜40% of genes.

In one embodiment, the present invention contemplates that measurement of a global methylation status represents the total amount of hypomethylation and hypermethylation that is present within the genome at any one time. In one embodiment, the present invention contemplates that deviations of a global methylation status from normal indicates the presence of, or development of, a disease state. For example, dramatic changes in the methylation of the genome have been shown to be a cause for the induction of neoplasias. DNA hypomethylation has been shown to be apparent in the very early stages of tumor progression prior to any observed tumor formation.

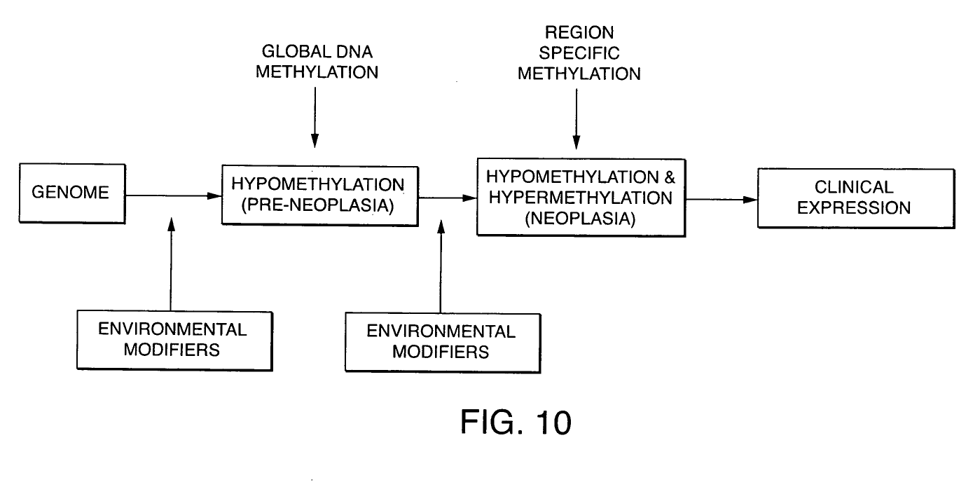

It is believed that an assay that is sensitive in detecting the changes in the methylation status of the genome, as it begins to alter into a potential disease state, would serve as an early warning detection system. FIG. 10.

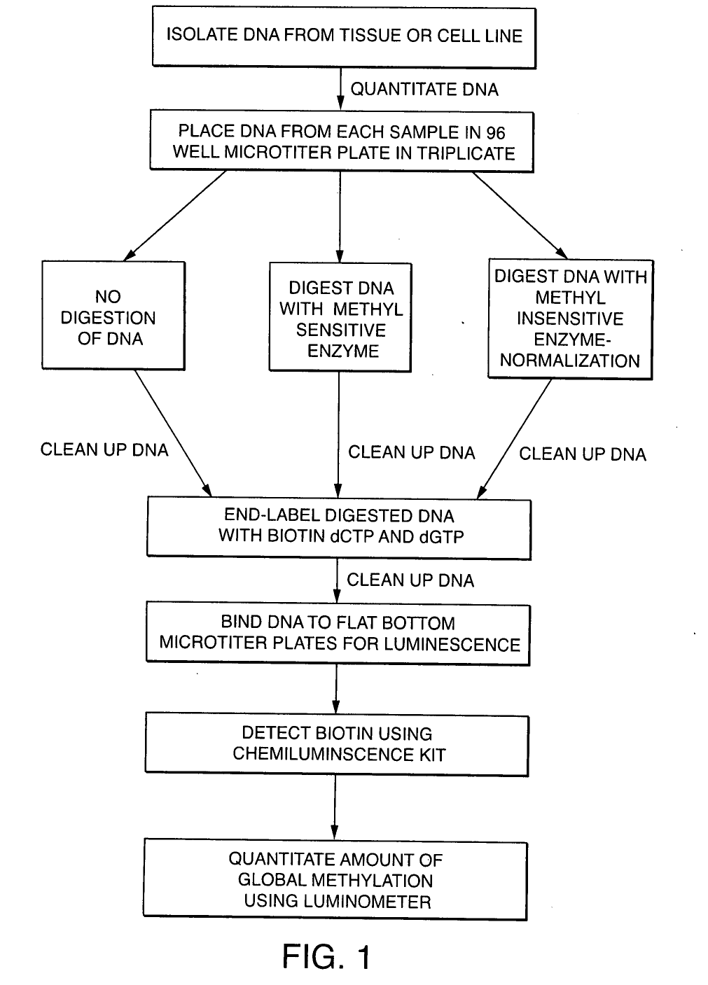

The GM-RESA assay can be performed in triplicate for each patient sample. In one embodiment, genomic DNA is aliquoted into each well of a multi-well plate such as a 96 well PCR plate. To prevent any down stream reactions occurring at 5′ or 3′ overhangs of the genomic DNA, which might have occurred due to shearing in the DNA isolation step, the genomic DNA may be end-filled to create blunt ends by incubation with a mixture of adenine, guanine, cytosine, and thymidine dideoxynucleotides. The blunt end-filled DNA can then purified using a Sephadex G50 columns. The purified DNA may then be digested with both methyl-sensitive and methyl-insensitive restriction enzymes, which results in DNA fragments cut at nucleic acid positions that are both non-methylated and methylated. The generated DNA fragments can then be end-filled with biotin-labeled dCTP and dGTP, such that the terminal ends have a biotin label. The DNA may then be adhered to a multi-well plate such as a 96 well white Microfluor 2 plate and the biotin label is detected using a commercially available Biotin Chemiluminescent Kit and quantitated by a luminometer. See FIG. 1

One utility of this assay may be to detect changes in DNA methylation. Such changes could signify a trend toward a disease state or an overall change in general health. The assay can be performed in a cost effective manner and in any hospital laboratory or central staging laboratory. This assay can be coupled to other assays, such as those described herein as examples that measure the changes in methylation of: i) novel DNA methylation biomarkers; or ii) known methylated genes that are associated with a particular disease. An assay of this type can be coupled to complement other diagnostic measures to further validate a physician's diagnosis. One example comprises the early diagnosis of lung cancer. In one embodiment, a method detecting global DNA methylation and specific lung cancer DNA methylation biomarkers would be performed prior to a Computerized Tomography (CT) scan (a costly procedure). In a further embodiment, the patient undergoes a CT scan when the methylation status is higher than normal. Another example would be the diagnosis of asthma where a spirometer or a methacholine challenge is performed. Appropriate diagnosis of this disease would place the patient on the correct therapeutic regime to reduce pulmonary loss over time. Further, an assay of this nature has added utility for prognosis and measurement of the efficacy of therapeutic treatment.

Also, this assay can be used as a screening method to determine overall genomic stability and thereby determine general well being. This might be an assay that would complement a regime of dietary supplements and determine overall genomic methylation levels and genome stability in an individual.

2. Current Methods of Detecting Global DNA Methylation

In one embodiment, the present invention contemplates methods that measure global DNA methylation. Methods to detect global DNA methylation have been reported that are based upon separating individual nucleotide bases from the genome to detect methyl cytosines using techniques including, but not limited to, quantitation through 5-methyl cytosine antibodies, radioactive labeling the CpG sites using SssI methyltransferase, digestion with methyl sensitive enzymes, and pyrosequencing of L1 elements. Each technique, however, has some specific disadvantages.

a. Separation and Quantitation of 5-Methyl Cytosines

One reported method enzymatically digests a genome using nuclease P1, DNase I in the presence of bacterial alkaline phosphatase, thereby generating deoxyribonucleosides (i.e., for example, adenosine, cytosine, thymidine, or guanosine). These four bases, as well as, 5-methyl cytosines may be separated by several techniques such as, but not limited to: i) reversed-phase high performance liquid chromatography (RP-HPLC), Kuo et al., Nucleic Acids Res 8:4763-4776 (1980); ii) two dimensional thin layer chromatography (2D-TLC), Wilson et al., Anal Biochem 152:275-284 (1986); iii) high performance liquid chromatography-mass spectrometry (HPLC-MS), Annan et al., J Chromatogr 465:285-96 (1989) and Friso et al., Proc Natl Acad Sci USA 99:5606-5611 (2002); iv) high performance capillary electrophoresis (HPCE), Fraga et al., Electrophoresis 23:1677-1681 (2002); and liquid chromatography-electrospray ionization-tandem mass spectrometry (LC-ESI-MS/MS), Song et al., Anal Chem 77:504-510 (2005). LC-ESI-MS/MS has been suggested as preferable for use with limited amounts of clinical samples. Disadvantages of these techniques include, but are not limited to, varying degrees of accuracy and reproducibility and require expensive equipment.

b. Quantitation of Radioactive Methyl Groups Incorporated into the Genome

One reported method incubates genomic DNA with radio-labeled S-adenosylmethionine (SAM) and methyltransferase SssI methylase. It is believed that SAM provides a source of methyl groups, and methyltransferase SssI methylase catalyzes the addition of SAM's radio-labeled methyl group to unmethylated cytosines. Radio-label methyl cytosines incorporated into the DNA is reported to be inversely proportional to the amount of methylation in the genome. Balaghi et al., Biochem Biophys Res Commun 193:1184-1190 (1993); and Oakeley et al., Biotechniques 27(4):744-752 (1999). One drawback to this method is that it has been shown to give variable results within the same sample despite the fact that the data may appear reproducible when comparing matched tumor and adjacent normal. Johanning et al, J Nutr 132:3814 S-3818S (2002).

c. Quantitation through the Use of a Monoclonal Antibody Specific for 5-Methyl Cytosine

One reported immunohistochemical assay uses a monoclonal antibody that has been raised against m5Cyt in rabbits. Sano et al, Proc Natl Acad Sci USA 77:3851-3585 (1980); and Reynaud et al, Cancer Lett 61:255-262 (1992). The antibody was used to detect immobilized digested DNA, where the bound m5Cyt antibody is incubated with goat anti-rabbit IgG labeled with 125I and visualized through autoradiography. In addition, this assay has been modified to determine global DNA methylation status by staining the DNA in the nucleus of fixed cells and monitoring the methylation patterns in tumor cells. Piyathilake et al, 75:251-258 (2000). This method is disadvantageous because it is labor intensive, requires expensive microscope equipment, and is optimized only when using fixed cells.

d. Quantitating Methylation at Line Elements

One method monitors changes in methylation status in Line 1 and other repetitive DNA elements that are generally heavily methylated. It has been suggested that these regions could serve as a surrogate marker for global DNA methylation. This assay utilizes sodium bisulphite to treat the genomic DNA, which converts unmethylated cytosines into thymidines. Polymerase chain reaction (PCR) primers are designed to the Line 1 elements and, following amplification, the resulting PCR products are sequenced. Sequences from normal and tumor cell are then compared for methylation differences within these regions of the genome. Yang et al., Nucleic Acids Res 32:e38 (2004). The disadvantage of this procedure is that it relies on treatment of the DNA with sodium bisulphite, a technique that can be difficult to control, thereby resulting in a lack of reproducibility.

e. Quantitation of Global DNA Methylation through the Use of Methyl Sensitive Enzymes And Radiolabeled Cytosine

One method uses the cytosine extension assay in conjunction with methyl sensitive enzymes, in particular HpaII (C↓CGG), to digest the DNA. If the CpG dyad in the HpaII restriction site is methylated then the enzyme is incapable of cutting the DNA. Conversely, if the CpG dyad at the restriction site is methyl free then the enzyme can cut the DNA. HpaII leaves a 5′GC overhang. A single base end-fill reaction is performed using radiolabeled ³H-dCTP. The radiolabeled product is bound to a Whatman DE-81 ion exchange filter, washed to remove the unincorporated nucleotide. The filter is dried and processed for scintillation counting. The amount of radiation-induced scintillation is reported to be directly proportional to the number of digested ends, which reflects the level of methylation at the restriction sites only. Pogribny et al., Biochem Biophys Res Commun 262:624-628 (1999). The disadvantage of this assay includes, but is not limited to, reliance on radioactivity, and use of a limited number of restriction enzymes whose cleavage site must have a guanine as the first base in the 5′ overhang, thereby allowing the incorporation of the radiolabeled ³H-dCTP.

3. GM-RESA Methyl Densitometry

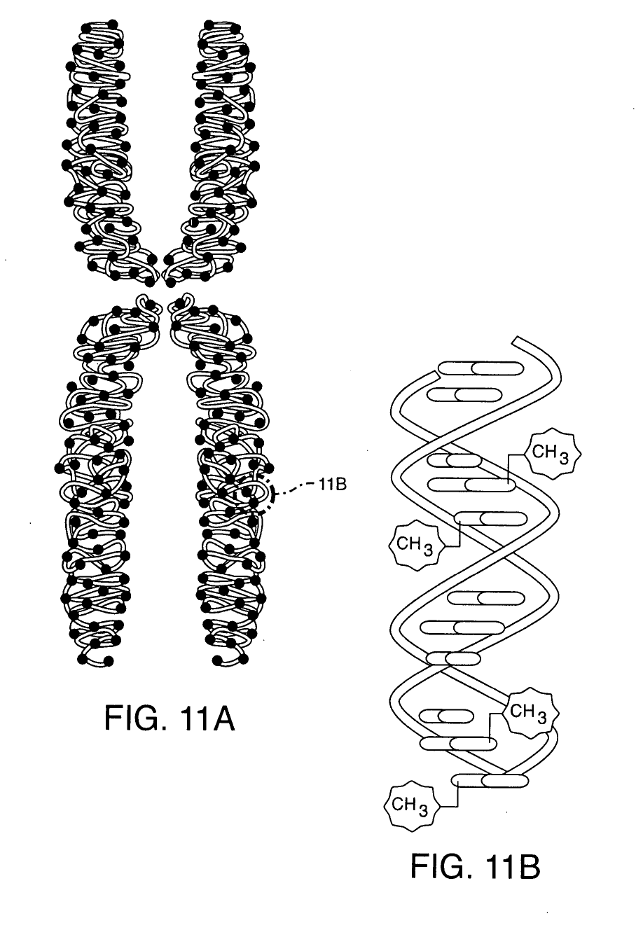

In one embodiment, the present invention contemplates a methyl densitometry method comprising determining the global density of DNA methylation. Although it is not necessary to understand the mechanism of an invention, it is believed that GM-RESA is an adaptation of the cytosine extension assay (supra). In one embodiment, the method comprises a methyl-densitometer (i.e., for example, a Global Methylation—REstriction Sensitive Assay (GM-RESA)) for measuring the density of genomic methylated CpG dyads. See, FIG. 11. In one embodiment, the method further comprises methyl sensitive enzymes, thereby generating nucleic acid fragments. In one embodiment, the method further comprises an end-fill reaction.

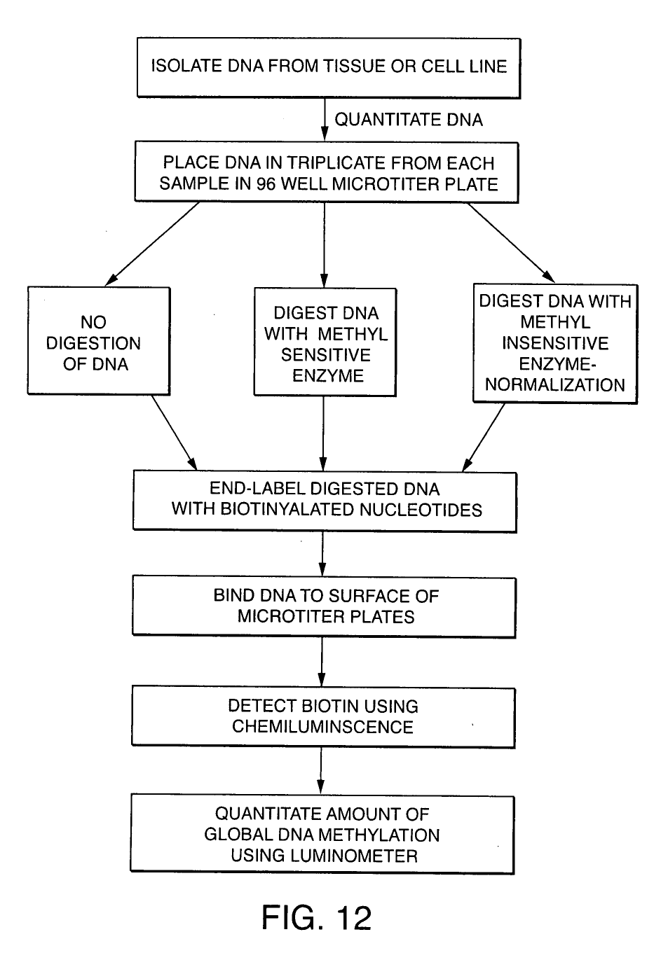

In one embodiment, the present invention contemplates a high-throughput methyl densitometry method comprising analyzing a plurality of nucleic acid samples on a microtiter plate. In one embodiment, the method comprises biotinylated (as opposed to radiolabeled) nucleotides. In one embodiment, the method comprises an analytical sensitivity of 5% (as compared to a 10% analytical sensitivity with radiolabeled assays). In one embodiment, the method utilizes 5 times less DNA per reaction than a radiolabeled assay. Specific advantages of a GM-RESA assay includes, but are not limited to, i) using off the shelf hardware; ii) easily applied technology; and iii) using standard laboratory equipment. These three advantages provide a GM-RESA assay which is cost effective and can be performed by any laboratory technician using standard molecular biology techniques. See, FIG. 12.

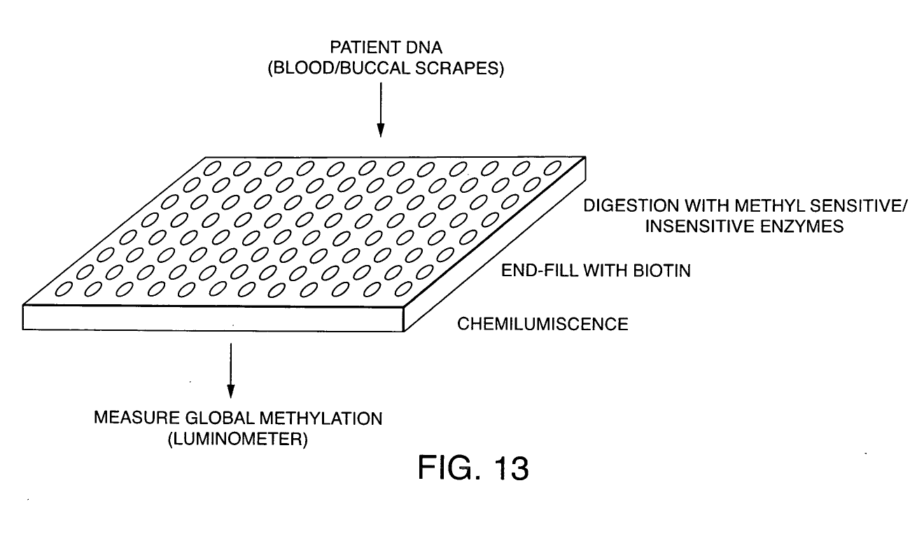

In one embodiment, the present invention contemplates a high-throughput GM-RESA method comprising standard multiwell reaction plates including, but not limited to, 96 or 384 wells per plate. See, FIG. 13. In one embodiment, the high-throughput method comprises a microtiter plate (i.e., for example, including 1536 wells per plate). In one embodiment, the high-throughput method comprises a microfluidic biochip. Although it is not necessary to understand the mechanism of an invention, it is believed that standard multiwell plates, microtiter plates, and/or microfluidic biochips allow multiple patient sample determination, thereby providing an advantage of automation.

In one embodiment, the present invention contemplates a GM-RESA method comprising using less than a 1 μg DNA sample. In one embodiment, the DNA is isolated from samples including, but not limited to, bodily tissue, blood, buccal swipes, or cell cultures.

In one embodiment, the present invention contemplates a GM-RESA method comprising a methyl sensitive enzyme and/or a methyl insensitive enzymes capable of digesting genomic DNA. In one embodiment, the digested DNA is end-filled with at least one biotinylated nucleotide selected from the group including, but not limited to, adenine, guanine, cytosine and thymidine. Although it is not necessary to understand the mechanism of an invention, it is believed that a specific biotinylated nucleotide combination is dependent upon the specific recognition site cleaved by a restriction enzyme. In one embodiment, a GM-RESA method may utilize any methyl sensitive enzyme (irrespective of where the CpG dyad lies within the restriction site) with a combination of a biotinylated adenine, a biotinylated guanine, a biotinylated cytosine, and a biotinylated thymidine. In one embodiment, an end-fill reaction may be performed following a restriction site cleavage by any methyl sensitive enzyme that leaves an end including, but not limited to, a 3′ overhang, a 5′ overhang, or a blunt end. In one embodiment, the method further comprises detecting the incorporated biotinylated nucleotides using a chemiluminescence kit wherein the assay readout is provided by a luminometer (i.e., measuring the amount of chemiluminescence emitted from each individual well of any multiwell plate; for example, a 96 or 384 Microfluor® 2 plate.

4. Application of Methylation Sensitive Restriction Enzymes to Measure Global DNA Methylation

Although it is not necessary to understand the mechanism of an invention, it is believed that the function of a methyl sensitive restriction enzyme in the context of this invention is in its inability to cut genomic DNA if it is methylated and its ability to cut genomic DNA if it is not methylated. It is further believed that other enzymes with this dual functionality represent a means to monitor the amount of methylation present in the genome.

In its healthy state, the human genome is believed 70% methylated through the addition of a methyl group at the 5′ position of the cytosine base. Therefore, one would expect that an application of methyl-sensitive enzymes to a healthy genome would produce few fragments because the methyl group at the cytosine base acts to inhibit the enzyme from cutting the DNA at that the restriction site, which contains a CpG dyad. A methyl-sensitive enzyme that has restriction sites that are uniformly scattered, with high frequency, throughout the genome, would serve as an excellent monitor of the methylation status of the CpG dyads. In turn this methyl-sensitive enzyme would represent a biomarker for global DNA methylation.

In one embodiment, the present invention contemplates a GM-RESA method comprising a methyl sensitive restriction enzyme, wherein the enzyme is HpaI (believed to cut at CCGG sites). In one embodiment, the method contemplates digesting a human genome at approximately 2.2 million HpaII sites. Although it is not necessary to understand the mechanism of an invention, it is believed that since HpaII has 14% of its sites within CpG islands, and 86% outside CpG islands, this enzyme would be very sensitive at monitoring methylation at both CpG dyads and the global genomic hypomethylation status. For example, a methylation index, quantitated by the accessibility of HpaII to digest DNA is disclosed herein as an indicator of methylation changes in the genome.

B. Global Methylation with CpG Island-Specific Primer Sets (GMSP)

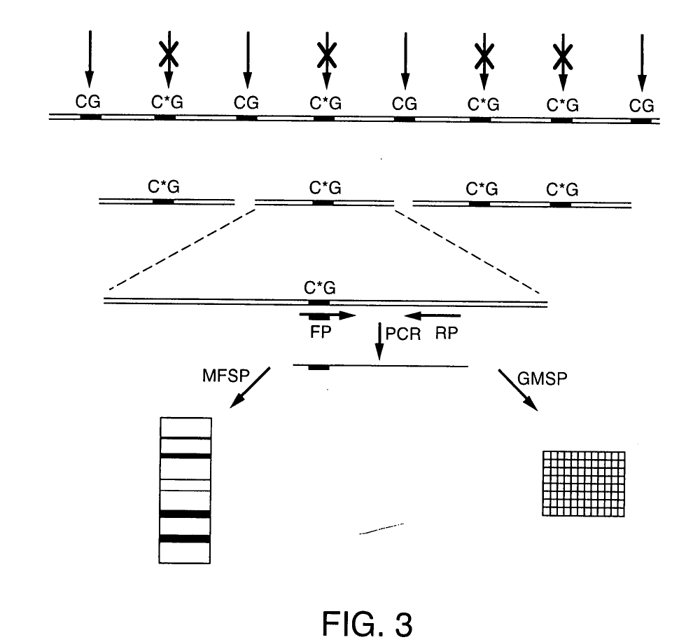

GMSP focuses on the methylation status of CpG Islands comprising three major steps. See FIG. 3.
Step 1: Genomic DNA is digested with a methylation specific, methylation sensitive and/or insensitive restriction enzyme.
Step 2: Digestion products from Step 1 are subject to a PCR amplification step by using PCR primers sets where the forward primer contains the restriction site. Consequently, all amplified PCR products have a 5′ terminal sequence matching the restriction site, therefore indicating a methylation event. The reverse primer is selected such that the size of the PCR products are easily handled by the following experimental steps.
Step 3: Total PCR product signal resulting from step 2 can be achieved by multiple methods including, but not limited to, incorporating fluorescence labeled primers, deoxyribonucleotide triphosphates, or DNA chelating dyes.

1. CpG Island-Specific Primer Construction

In one embodiment, the selection primer sets are strongly biased towards CpG Islands. However, the methods described herein are not limited to CpG Islands and can be applied for selecting primer sets that are biased towards other regions with known sequence characteristics. For example, primer sets have been selected comprising 10 nucleotides. It is not intended that the present invention be limited to primers comprising 10 nucleotides because primers of between 15-25 nucleotides may also be easily constructed by the methods described herein.

In one embodiment, the selection of CpG Island-specific primers includes calculating the frequencies of which all possible combinations of nucleotide sequences that are ten (10) nucleic acids in length, occurring either inside and outside CpG Islands in the human genome. For example, in one embodiment such frequencies were calculated using a custom computer program (Java based). For example, the human genomic sequence can be downloaded from the National Center for Biotechnology Information (NCBI) wherein the CpG Island boundaries can be determined using a CpG Report program (emboss.sourceforge.net). Briefly, the human genomic sequence would then be scanned using a “10 nucleotide window” on both strands. All “10 nucleotide windows” were noted and their frequencies updated during the scanning. When a “10 nucleotide window” was completely within the boundaries defined by the CpG Report it was considered as inside a CpG Island.

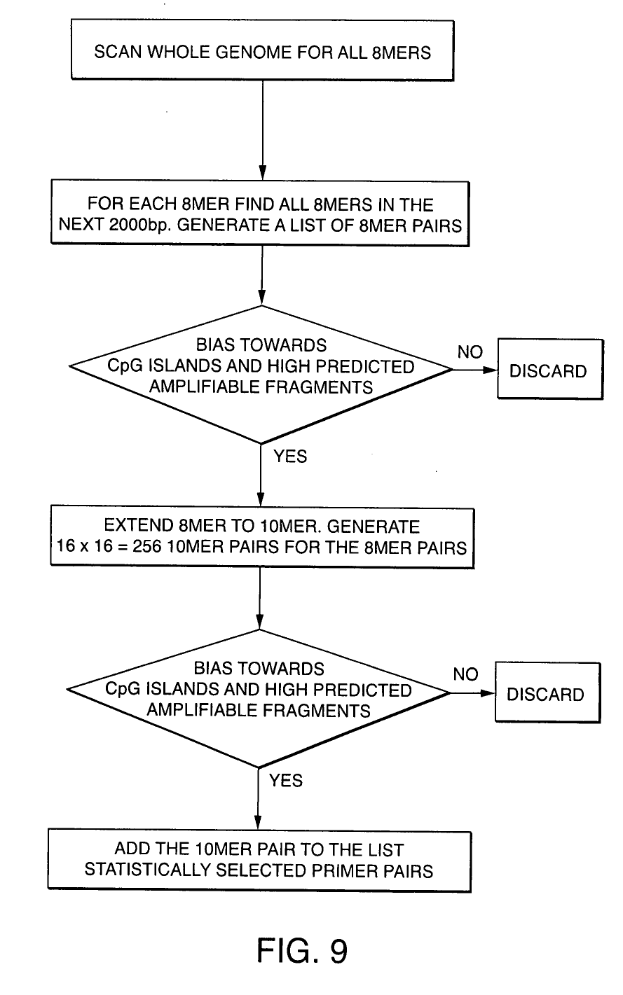

In a further embodiment, a heuristic approach was applied to get a set of oligonucleotide pairs that selectively amplify the CpG Islands using a custom Java-based computer program. See FIG. 9. First, all 8mers matching the genome were found. Then, for each of these 8mers, the preceding 2000 bp were scanned for all possible 8mers and the frequencies of all such 8mer oligonucleotide pairs were determined. A subset was selected from all the 8mer pairs that were appearing more often inside than outside the CpG Islands. In one embodiment, the 8mer frequency of appearance inside the CpG Island is greater than 500.

In another embodiment, each 8mer pair appearing inside the CpG Island was extended in both directions to generate all possible 10mers that completely contained the initial 8mer sequence. In the same manner as for the 8mers, the frequency of appearance within the CpG Island for all such 10mer pairs were calculated.

In one embodiment, improved primer oligonucleotides were identified that were better candidates for PCR amplification. In one embodiment, an improved 10mer primer pair has no more than 7 G's or 7 C's. In one embodiment, improved primer oligonucleotides comprises at least 85,825 10mer pairs.

As the number of all possible pairs of 10mers is virtually limitless, 2000 10mers were randomly chosen to form a 1000×1000 pairing matrix as an illustrative example. Among these chosen 2000 primers, 1000 were randomly selected from the population of all possible 10mers, and the other 1000 from a population of 10mers comprising CG dinucleotides. It should be noted that similar results would be expected for any other combination of similarly chosen 2000 10mers.

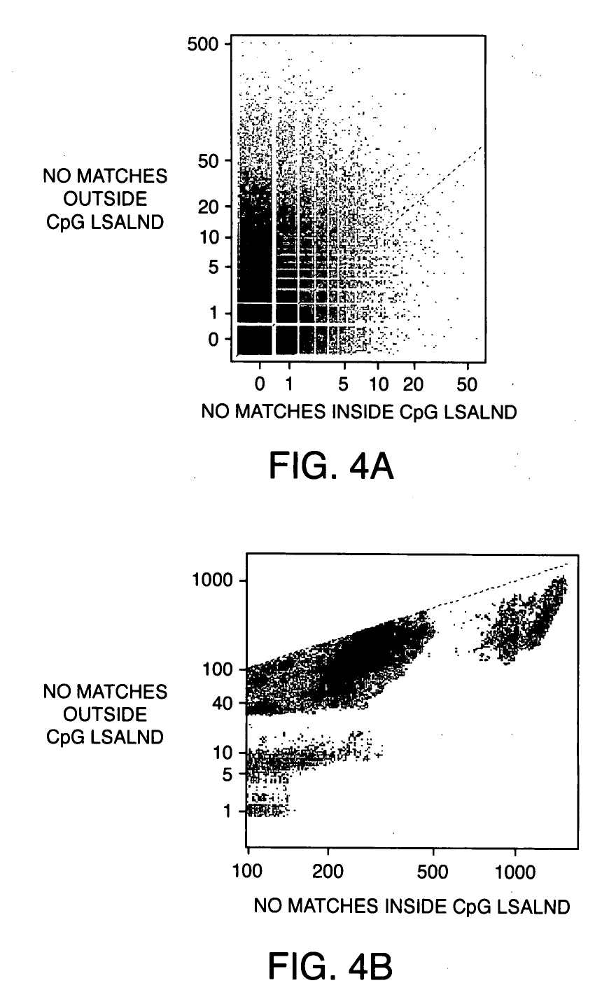

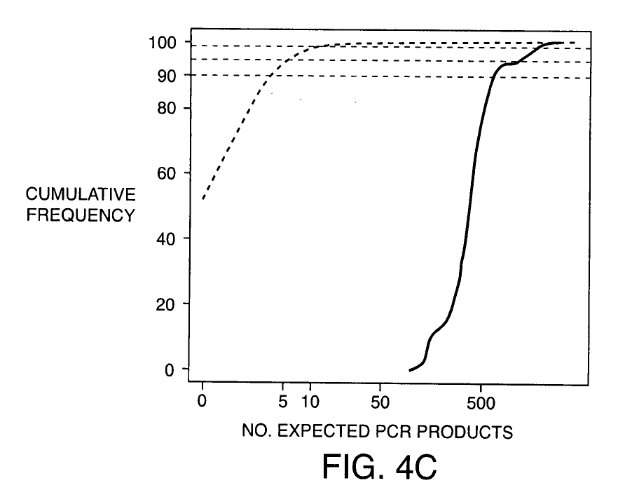

These randomly selected primer pairs were compared with CG dinucleotide primer pairs for the number of matches inside and outside of CpG Island. See FIGS. 4A & 4B. Further, the cumulative frequencies for the number of PCR fragments amplified by each type of primer pair was also calculated. See FIG. 4C.

As expected, random10mer primer pairs mostly amplify non-CpG Island sequences. See FIG. 4A. Further, the number of amplified PCR fragments from random primer pairs is low. See FIGS. 4C & 4B. It was further observed that more than half of the random primer pairs do not generate any PCR products when limited to a sequence length of less than 2 kb.

Ninety nine percent of the random primer pairs amplified less than 15 fragments while 99.9% amplified less than 64 fragments. See FIG. 4C-dashed lines. In terms of CpG Islands, only about 2.7% of the random primer pairs amplified a greater number of CpG Island fragments than PCR fragments generated from outside the CpG Island. Further, only 71 pairs of the 2.7% amplified more than 20 CpG Island fragments and only 2 random primer pairs amplified more than 50 fragments out of all 1 million possible pairing sets (i.e., 1000×1000). This statistical approach greatly enriches CpG Island selectivity and significantly increased the number of fragments that can be amplified.

In another embodiment, improved primer oligonucleotides were selected from primer pairs having specific cutting sites for methylation sensitive endonucleases. For example, 100 exemplary primer pairs are listed below in Table 1. In one embodiment, the primer sets are based on their degree of bias towards CpG Islands, the expected number of PCR products, and the size of PCR products. In one embodiment, CpG Island-specific primer pairs generate more multiple PCR products than random arbitrary PCR primers. See FIG. 4.

TABLE 1

Examples of CpG Island Specific Primer Pairs.

| | | | |
|---|---|---|---|
| | | Match | Match |
| | | Outside | Inside |
| Forward | Reverse | CpG | CpG |
| Primer | Primer | Island | Island |

| | | | |
|---|---|---|---|
| CCCGGCTAAA | TTTTTTGAGA | 446 | 1300 |
| (SEQ ID NO: 1) | (SEQ ID NO: 2) | | |
| GCTAAAACGG | GACTACAGGC | 315 | 1261 |
| (SEQ ID NO: 3) | (SEQ ID NO: 4) | | |
| CGGCTAAAAC | ACGGGGTTTC | 230 | 1143 |
| (SEQ ID NO: 5) | (SEQ ID NO: 6) | | |
| CTCTGTCGCC | CTGCAGTCCG | 381 | 901 |
| (SEQ ID NO: 7) | (SEQ ID NO: 8) | | |
| AAGCTCCGCT | GTGGATCATG | 177 | 889 |
| (SEQ ID NO: 9) | (SEQ ID NO: 10) | | |
| AAGCGCAAGG | GTCTGTGCCC | 313 | 463 |
| (SEQ ID NO: 11) | (SEQ ID NO: 12) | | |
| GCGAGGCATT | CACTGATGGG | 238 | 429 |
| (SEQ ID NO: 13) | (SEQ ID NO: 14) | | |
| GGGAAGCGCA | ATCGTCTGAA | 207 | 392 |
| (SEQ ID NO: 15) | (SEQ ID NO: 16) | | |
| TGGGAAGCGC | TCCTGAATCT | 130 | 376 |
| (SEQ ID NO: 17) | (SEQ ID NO: 18) | | |
| GCGAGCCGAA | TCACCCCTTT | 190 | 371 |
| (SEQ ID NO: 19) | (SEQ ID NO: 20) | | |
| GAGCGACGCA | TGTTATGTGT | 262 | 339 |
| (SEQ ID NO: 21) | (SEQ ID NO: 22) | | |
| GCCGGGATTG | GTGATGACTC | 214 | 328 |
| (SEQ ID NO: 23) | (SEQ ID NO: 24) | | |
| GAGCGACGCA | ATACATTCTT | 261 | 328 |
| (SEQ ID NO: 25) | (SEQ ID NO: 26) | | |
| CGACGCAGAA | TGTGCCCCTG | 197 | 322 |
| (SEQ ID NO: 27) | (SEQ ID NO: 28) | | |
| GGTGACGGAC | TTTTCAAAGT | 148 | 317 |
| (SEQ ID NO: 29) | (SEQ ID NO: 30) | | |
| TTCCGAGTCA | CCTTGGTTTT | 128 | 315 |
| (SEQ ID NO: 31) | (SEQ ID NO: 32) | | |
| TCCCTTTCCG | TGCAACCCCT | 106 | 309 |
| (SEQ ID NO: 33) | (SEQ ID NO: 34) | | |
| ACGCCTGACT | TGGCGGATCA | 112 | 309 |
| (SEQ ID NO: 35) | (SEQ ID NO: 36) | | |
| CCCTTTCCGA | GCCCTTAACA | 116 | 308 |
| (SEQ ID NO: 37) | (SEQ ID NO: 38) | | |
| TTCCGAGTCA | TATGATGTTA | 119 | 306 |
| (SEQ ID NO: 39) | (SEQ ID NO: 40) | | |
| TTTCCGAGTC | GAGTATCTTT | 123 | 303 |
| (SEQ ID NO: 41) | (SEQ ID NO: 42) | | |
| GTGACGGACG | GAAATTCTGG | 108 | 303 |
| (SEQ ID NO: 43) | (SEQ ID NO: 44) | | |
| CACGCCTGAC | AACCATCCGA | 116 | 303 |
| (SEQ ID NO: 45) | (SEQ ID NO: 46) | | |
| GTGCCGGGAT | CGCCCTTAAT | 169 | 299 |
| (SEQ ID NO: 47) | (SEQ ID NO: 48) | | |
| GTGACGGACG | TACATTCTTC | 97 | 296 |
| (SEQ ID NO: 49) | (SEQ ID NO: 50) | | |
| TCCCACCCGA | CCGAGAGATC | 234 | 295 |
| (SEQ ID NO: 51) | (SEQ ID NO: 52) | | |
| TGGGAAGCGC | AGGCGCTCTG | 82 | 294 |
| (SEQ ID NO: 53) | (SEQ ID NO: 54) | | |
| CAATCGCAGG | CCACGGTCTC | 132 | 294 |
| (SEQ ID NO: 55) | (SEQ ID NO: 56) | | |
| AAATCGGGTC | TAGCGCTTCC | 207 | 291 |
| (SEQ ID NO: 57) | (SEQ ID NO: 58) | | |
| CGTCACCCCT | GCTCCGGTCT | 122 | 286 |
| (SEQ ID NO: 59) | (SEQ ID NO: 60) | | |
| CCGCGAGTGA | TGTTTGTGTC | 165 | 286 |
| (SEQ ID NO: 61) | (SEQ ID NO: 62) | | |
| TTAAGCCGGT | CACTAGGGAG | 112 | 282 |
| (SEQ ID NO: 63) | (SEQ ID NO: 64) | | |
| GCGACGCAGA | CTTTGTGGCG | 273 | 280 |
| (SEQ ID NO: 65) | (SEQ ID NO: 66) | | |
| GACGCACCTG | CATCAGCTCC | 106 | 279 |
| (SEQ ID NO: 67) | (SEQ ID NO: 68) | | |
| CCGAGTCAAA | AATCAGACGT | 96 | 276 |
| (SEQ ID NO: 69) | (SEQ ID NO: 70) | | |
| GACGCACCTG | TTGATCCTGT | 103 | 274 |
| (SEQ ID NO: 71) | (SEQ ID NO: 72) | | |
| AGTCTCGTTC | GAGACGCTCC | 118 | 274 |
| (SEQ ID NO: 73) | (SEQ ID NO: 74) | | |
| TTCCGAGTCA | GTCTGAAGCC | 107 | 272 |
| (SEQ ID NO: 75) | (SEQ ID NO: 76) | | |
| CACCCGAATA | CTGCCCGTTC | 203 | 271 |
| (SEQ ID NO: 77) | (SEQ ID NO: 78) | | |
| TCCCGAGGTG | CCTTCCGCAG | 166 | 270 |
| (SEQ ID NO: 79) | (SEQ ID NO: 80) | | |
| TGCAGACGGA | TGGGATGGCG | 135 | 268 |
| (SEQ ID NO: 81) | (SEQ ID NO: 82) | | |
| TGCCTGCGAT | TCAATGAGCT | 134 | 266 |
| (SEQ ID NO: 83) | (SEQ ID NO: 84) | | |
| GACGCACCTG | ATGTTAGCTG | 95 | 266 |
| (SEQ ID NO: 85) | (SEQ ID NO: 86) | | |
| CCACCCGAAT | GATCGCATCG | 185 | 266 |
| (SEQ ID NO: 87) | (SEQ ID NO: 88) | | |
| GGGTGACGGA | CGTAGGACCC | 130 | 265 |
| (SEQ ID NO: 89) | (SEQ ID NO: 90) | | |
| GACGCACCTG | GGTGTCAGTG | 96 | 260 |
| (SEQ ID NO: 91) | (SEQ ID NO: 92) | | |
| CCTTTCCGAG | ACTGCGTTCC | 98 | 258 |
| (SEQ ID NO: 93) | (SEQ ID NO: 94) | | |
| CTAACCGCGA | CAGTAGGGGC | 103 | 255 |
| (SEQ ID NO: 95) | (SEQ ID NO: 96) | | |
| GACCGGCTTA | GGTCTTTTCA | 125 | 253 |
| (SEQ ID NO: 97) | (SEQ ID NO: 98) | | |
| AACCGCGAGT | AGTCTCCCAT | 109 | 253 |
| (SEQ ID NO: 99) | (SEQ ID NO: 100) | | |
| CCGAATATTG | GATGTCCTTT | 97 | 249 |
| (SEQ ID NO: 101) | (SEQ ID NO: 102) | | |
| TGGTTTTCGT | CCCAGACGAT | 150 | 248 |
| (SEQ ID NO: 103) | (SEQ ID NO: 104) | | |
| CGAGTGCCTG | AGATCAACAG | 110 | 248 |
| (SEQ ID NO: 105) | (SEQ ID NO: 106) | | |
| ACCGGCTTAA | TCTCAGATCT | 129 | 248 |
| (SEQ ID NO: 107) | (SEQ ID NO: 108) | | |
| ACCGGCTTAA | TGATTTTGCA | 126 | 247 |
| (SEQ ID NO: 109) | (SEQ ID NO: 110) | | |
| ACCGCGAGTG | GAGCATGCTG | 138 | 244 |
| (SEQ ID NO: 111) | (SEQ ID NO: 112) | | |
| GACCGGCTTA | GATACCCTTT | 118 | 243 |
| (SEQ ID NO: 113) | (SEQ ID NO: 114) | | |
| GACCGGCTTA | ACAGATGGGT | 121 | 241 |
| (SEQ ID NO: 115) | (SEQ ID NO: 116) | | |
| CGAGTGCCTG | CCGCCCTTAA | 145 | 241 |
| (SEQ ID NO: 117) | (SEQ ID NO: 118) | | |
| ACGGACGCAC | TGCAGAGGTT | 53 | 241 |
| (SEQ ID NO: 119) | (SEQ ID NO: 120) | | |
| GGACGCACCT | ATATTGTTAT | 81 | 240 |
| (SEQ ID NO: 121) | (SEQ ID NO: 122) | | |
| CAATCCCGGC | ATGCCGAGCC | 98 | 240 |
| (SEQ ID NO: 123) | (SEQ ID NO: 124) | | |
| TCCCTTTCCG | CTCGATGGTC | 89 | 238 |
| (SEQ ID NO: 125) | (SEQ ID NO: 126) | | |
| CTTTCCGAGT | CGTGGGCGTA | 74 | 238 |
| (SEQ ID NO: 127) | (SEQ ID NO: 128) | | |
| ACGGACGCAC | GAGACTAGGA | 66 | 237 |
| (SEQ ID NO: 129) | (SEQ ID NO: 130) | | |
| ATTGCGCTTT | CACATAGTCC | 115 | 236 |
| (SEQ ID NO: 131) | (SEQ ID NO: 132) | | |
| CTCGCGGTTA | GGGGTTTCGC | 103 | 235 |
| (SEQ ID NO: 133) | (SEQ ID NO: 134) | | |
| AACCGCGAGT | CTCACTTTCC | 105 | 234 |
| (SEQ ID NO: 135) | (SEQ ID NO: 136) | | |
| GGTGACGGAC | TCTGCACGTG | 80 | 232 |
| (SEQ ID NO: 137) | (SEQ ID NO: 138) | | |
| GACGGACGCA | GCTTGGTAGA | 53 | 232 |
| (SEQ ID NO: 139) | (SEQ ID NO: 140) | | |
| ACCCGAATAT | GAGCCTATGT | 92 | 232 |
| (SEQ ID NO: 141) | (SEQ ID NO: 142) | | |
| ATTGCGCTTT | CTTTGAGGGT | 103 | 231 |
| (SEQ ID NO: 143) | (SEQ ID NO: 144) | | |
| TATTGCGCTT | GGGAGAACCA | 128 | 230 |
| (SEQ ID NO: 145) | (SEQ ID NO: 146) | | |
| CGGTTAGGAG | GTGTTGGCCG | 118 | 230 |
| (SEQ ID NO: 147) | (SEQ ID NO: 148) | | |
| ACCGGCTTAA | GTCTCTGCAC | 103 | 226 |
| (SEQ ID NO: 149) | (SEQ ID NO: 150) | | |
| AAAGCCGCGG | CATTCTGATT | 48 | 226 |
| (SEQ ID NO: 151) | (SEQ ID NO: 152) | | |
| CGAATATTGC | TGATGGGTCT | 93 | 225 |
| (SEQ ID NO: 153) | (SEQ ID NO: 154) | | |
| GACCGGCTTA | GTAGTTCTCG | 106 | 223 |
| (SEQ ID NO: 155) | (SEQ ID NO: 156) | | |
| ACCCGAATAT | CGTTGGCCTG | 78 | 218 |
| (SEQ ID NO: 157) | (SEQ ID NO: 158) | | |
| ATCCCGGCAC | TTTCCACGGT | 114 | 211 |
| (SEQ ID NO: 159) | (SEQ ID NO: 160) | | |
| TGACGGACGC | GTTGATCGCA | 56 | 208 |
| (SEQ ID NO: 161) | (SEQ ID NO: 162) | | |
| CAGACCGGCT | AGCAAGCCTG | 70 | 207 |
| (SEQ ID NO: 163) | (SEQ ID NO: 164) | | |
| GACGGACGCA | AGAGATCCGC | 50 | 205 |
| (SEQ ID NO: 165) | (SEQ ID NO: 166) | | |
| ATCGCAGGCA | TTCCACGGTC | 93 | 198 |
| (SEQ ID NO: 167) | (SEQ ID NO: 168) | | |
| ATTGCGCTTT | GGCTGGTACC | 81 | 189 |
| (SEQ ID NO: 169) | (SEQ ID NO: 170) | | |
| GGATCACTCG | GTTTTCGTAT | 85 | 187 |
| (SEQ ID NO: 171) | (SEQ ID NO: 172) | | |
| ACCACGAGAC | ACCCGACCTT | 54 | 186 |
| (SEQ ID NO: 173) | (SEQ ID NO: 174) | | |
| AAAGCCGCGG | CTGGGTTCTT | 9 | 184 |
| (SEQ ID NO: 175) | (SEQ ID NO: 176) | | |
| GGTTTTCGTA | GAAGAGGCGC | 115 | 168 |
| (SEQ ID NO: 177) | (SEQ ID NO: 178) | | |
| AAAAGCCGCG | CTTAGTTAAC | 7 | 157 |
| (SEQ ID NO: 179) | (SEQ ID NO: 180) | | |
| CGGCTTAAAA | TTCTAGTTAT | 123 | 146 |
| (SEQ ID NO: 181) | (SEQ ID NO: 182) | | |
| CCGGCTTAAA | GATCTCAGAC | 111 | 143 |
| (SEQ ID NO: 183) | (SEQ ID NO: 184) | | |
| AAAGCCGCGG | ATTACAATGA | 6 | 143 |
| (SEQ ID NO: 185) | (SEQ ID NO: 186) | | |
| CCGCGGCTTT | AAAGTCGCGG | 6 | 140 |
| (SEQ ID NO: 187) | (SEQ ID NO: 188) | | |
| CGAGCCGAAG | TTGTTCTGTT | 53 | 135 |
| (SEQ ID NO: 189) | (SEQ ID NO: 190) | | |
| GAAGCGCAAG | CGGTGTAGAT | 31 | 134 |
| (SEQ ID NO: 191) | (SEQ ID NO: 192) | | |
| CCTTTCCGAG | TCGTCAAAGT | 85 | 133 |
| (SEQ ID NO: 193) | (SEQ ID NO: 194) | | |
| CAGACCGGCT | AGATGTCCTT | 35 | 130 |
| (SEQ ID NO: 195) | (SEQ ID NO: 196) | | |
| AAAGCCGCGG | TTCATGAGCC | 6 | 126 |
| (SEQ ID NO: 197) | (SEQ ID NO: 198) | | |
| AAAGCCGCGG | TCGTCCACAA | 6 | 124 |
| (SEQ ID NO: 199) | (SEQ ID NO: 200) | | |

2. Oligonucleotide Ligation

Chemically synthesized oligonucleotides typically are obtained without a 5′ phosphate. The 5′ ends of such oligonucleotides are not substrates for phosphodiester bond formation by ligation reactions that employ DNA ligases typically used to form recombinant DNA molecules. Where ligation of such oligonucleotides is desired, a phosphate can be added by standard techniques, such as those that employ a kinase and ATP. The 3′ end of a chemically synthesized oligonucleotide generally has a free hydroxyl group and, in the presence of a ligase, such as T4 DNA ligase, readily will form a phosphodiester bond with a 5′ phosphate of another polynucleotide, such as another oligonucleotide. This reaction can be prevented selectively, where desired, by removing the 5′ phosphates of the other polynucleotide(s) prior to ligation.

3. Methyl Group Detection

Many different technologies can detect the total 5-methylcytosine DNA level in the whole genome including, but not limited to, High-performance Liquid Chromatograph (HPLC) and High-performance Capillary Electrophoresis. Singer J., J. Biol. Chem. 252:5509 (1997); and Fraga et al., Electrophoresis 21:2990(2000), respectively. Very few technologies, however, exist which can detect total regional methylation (i.e. for example, CpG Island methylation) for the whole genome.

Two methods suggested for detecting total regional methylation in the whole genome use either region biased restriction enzyme (i.e., for example, BssHII) or PCR primers against known repetitive sequences (i.e., for example, Alu or LINE). Pogribny et al., BBRC 262:624-628 (1999); and Yang et al., Nucleic Acids Res. 32:e38 (2004), respectively. The GMSP methodology takes advantage of aspects of both techniques, utilizing both restriction endonucleases and PCR amplification. Although it is not necessary to understand the mechanism of an invention, it is believed that a methylation sensitive enzyme, as used in GMSP, will distinguish between methylated and unmethylated restriction sites and only cleave an unmethylated restriction site.

PCR primers, on the other hand, were designed to selectively amplify regions of the genome that were of interest. The combination of the methylation sensitive restriction enzyme and the genome-wide specificity of CpP Island specific PCR primers provides a method to obtain information representative of the methylation status within the whole genome. Further, this technique can be used to scan for point mutations and deletions/insertions.

One embodiment of the present invention comprises GMSP that provides a framework which can be optimized for specific purposes. For example, the primer sets can be made longer than described above for GMSP so the amplification will be more specific. Alternatively, the primer can be made to be more degenerate so a higher coverage of the genome can be achieved. A further optimization embodiment comprises a reverse primer which also contains methylation sensitive enzyme cutting sites, thereby allowing amplification of only those fragments with contain at least two methylated cytosines (i.e., one from the forward primer and the other from the reverse primer).

C. Methylation in Specific Region Quantitation (MSRquant)

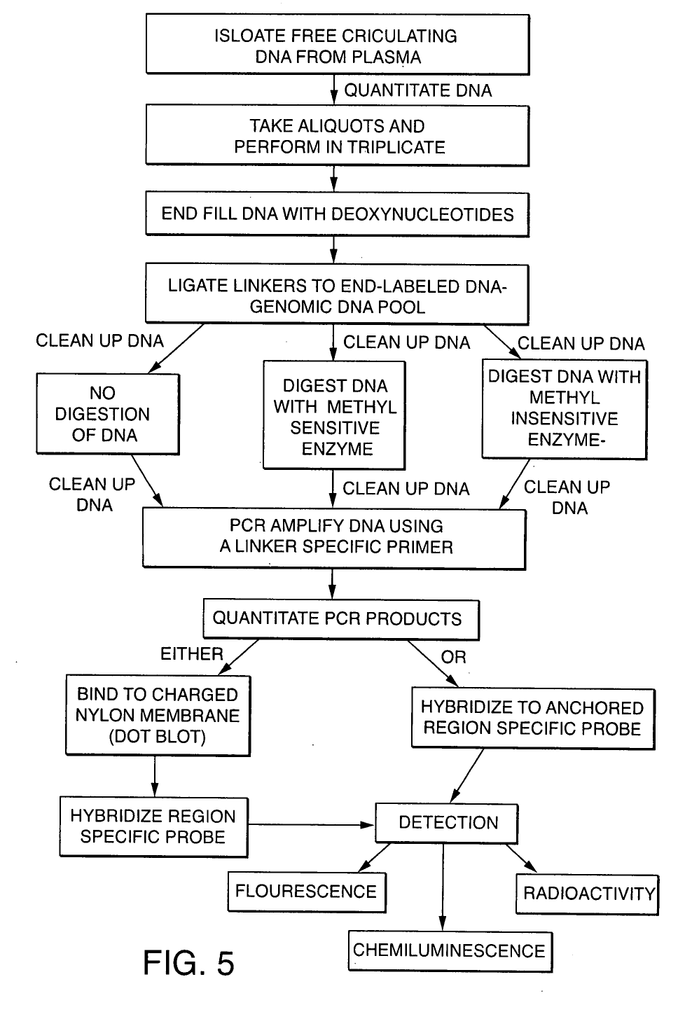

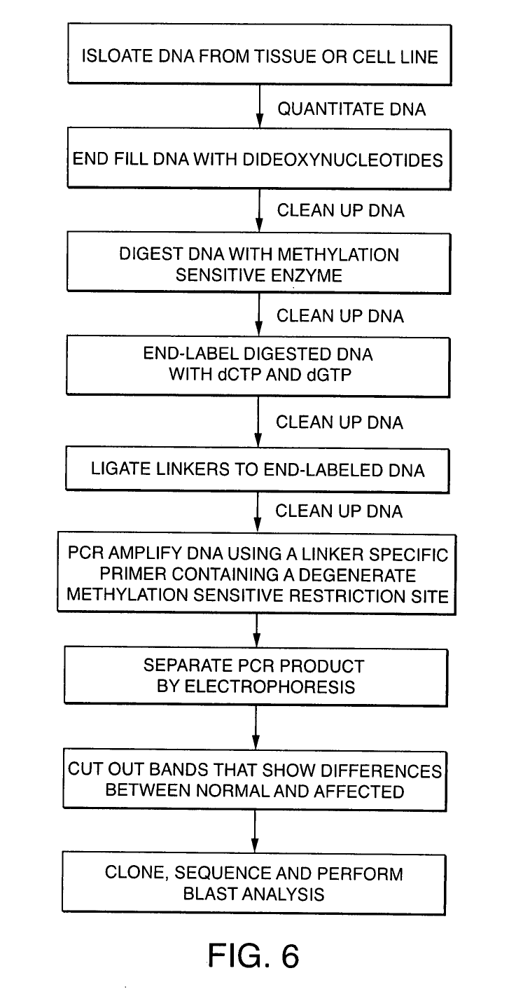

In one embodiment, MSRquant improves the detection of methylated DNA using freely circulating DNA isolated from patient plasma. See FIG. 5. In one embodiment, the procedure is performed in triplicate.

In one embodiment, the freely circulating DNA (obtained from any biological sample) is end-filled with a mixture of adenine, guanosine, cytosine, and thymidine dideoxynucleotides to generate blunt end fragments. The end-filled DNA is ligated to a double stranded oligonucleotide linker, which contains a unique sequence that is not homologous to any DNA sequence within the human genome. Prior to ligation of the linker to the DNA, the single stranded oligonucleotides, which are complementary to each other, re-annealed together. After the ligation reaction the DNA is cleaned to remove any excess linker. The product represents a non-amplified freely circulating DNA pool.

In a further embodiment, a first aliquot of freely circulating pool DNA is digested with a methyl sensitive enzyme, a second aliquot is digested with a methyl insensitive enzyme, and a third aliquot is not digested with any enzyme. After the digestion is complete, the digested and undigested DNA pools are cleaned (i.e., for example, using Shephadex® filtration). A PCR primer that matches the sequence of the linker is utilized to PCR amplify the DNA pools. An aliquot of the digested and undigested DNA pools are PCR amplified.

The PCR products are anchored to a charged nylon membrane to generate a dot blot and a gene specific probe is hybridized to the filter. Probes are designed to hybridize to novel DNA methylation biomarkers associated with a disease or nucleic acid probes designed to hybridize to known methylated regions of the genome that are associated with a disease. The nucleic acid probes can be tagged with a fluorescent marker, a biotin molecule or radioactive label. Detection is by a fluorometer, luminometer (chemiluminesce) and film, respectively.

In an alternative embodiment, the PCR products are hybridized to gene-specific oligonucleotides. The gene-specific oligonucleotides are anchored to the surface of a 96-well plate by any moiety having an affinity for the microtiter dish surface (i.e., for example, an amine group). Hybridization is performed in the well and only the fragments that are homologous to the anchored oligonucleotides are captured. In one embodiment, the captured products are washed and detected by chemiluminescence, fluorescence or radioactivity where the PCR products have been tagged by biotin, fluorescence or a ³²P-label at the 5′ end of the PCR primer.

The MSRquant assay can determine the level of methylation in any specific region of the genome. In particular, MSRquant complements GMSP by lending specificity, thereby determining the degree of methylation throughout the genome. Further, MSRquant is cost effective and may be performed in any hospital laboratory or central staging laboratory. The combination of MSRquant and GMSP constitutes an approach for probing global methylation (sensitivity) and specific regions of the genome (specificity) that are associated with any complex disease.

III. Methods to Detect DNA Methylation Biomarkers

A. Methylation Sensitive Amplification System (MESAS)

MESAS may be useful for the identification of novel DNA methylation biomarkers that are specifically associated with a disease. MESAS can diagnose any disease by comparing DNA between normal versus an affected individuals, or by comparing DNA between normal versus diseased tissue from the same individual. One example of such an application would be cancer where there is a normal part of the tissue and diseased part.

In one embodiment, DNA is non-invasively collected (i.e., for example, using a blood sample or buccal swab). In one embodiment, asthma is diagnosed without a lung tissue sample. In one embodiment, changes in DNA methylation patterns provides a method to discover novel DNA methylation biomarkers that can be used to clearly diagnose disease (i.e., for example, asthma).

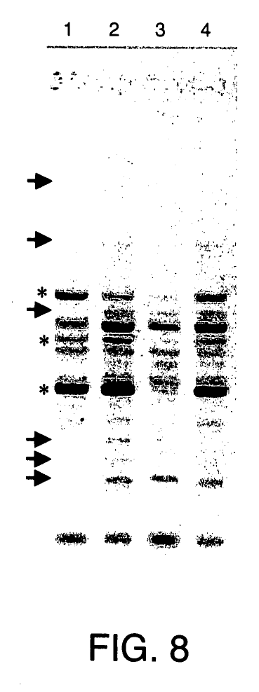

In one embodiment, MESAS may utilize genomic DNA isolated from cell lines or organic tissues. See FIG. 8. Further, genomic DNA may be collected from sources including, but not limited to, tissues, blood, stool, spinal fluid, saliva, urine, buccal and other bodily fluids.

After genomic DNA is isolated (supra), down stream reactions may be prevented from occurring at 5′ or 3′ overhangs (possibly occurring due to shearing) by end-filling with adenine, guanosine, cytosine, and thymidine dideoxynucleotides. These blunt end DNA fragments are then cleaned (i.e., for example, by Sephadex® filtration) and then digested with a methyl specific enzyme (i.e., for example, BisI), which will cut DNA only at methylated cytosines. This is followed by an end-fill reaction with adenine, guanosine, cytosine, and thymidine dideoxynucleotides which add a single nucleotide to the DNA restriction fragments.

The end-filled DNA restriction fragment is ligated to a double stranded oligonucleotide linker, which contains a unique sequence that is not homologous to any DNA sequence within the human genome. The linker also contains an EcoRI restriction site to clone fragments into an EcoRI linearized vector. Prior to linker ligation to the DNA restriction fragment, the single stranded oligonucleotide, which are complementary to each other, are annealed together. After the ligation reaction, the DNA is cleaned (i.e., for example, by Sephadex® filtration) to remove any excess linker.

An aliquot of the ligated product is then PCR amplified wherein each PCR reaction contains a first 5′ primer that is complementary to a linker sequence and a 3′ primer that is complementary to the methylation sensitive restriction site followed by two degenerate bases.

The PCR products may be separated by 4% to 20% gradient polyacrylamide gel electrophoresis. Differences in band intensity or presence or absence of bands are quantitatively scored. The fragments are cut out of the gel, crushed and the DNA eluted using elution buffer. The separated DNA bands are ethanol precipitated and cloned into a vector for propagation into an E. Coli host using standard molecular biology techniques. The cloned fragments are sequenced (Agencourt) and the sequences are compared against the GenBank database by BLAST analysis to identify the location within the human genome that the fragments originate from.

B. Methylation Fingerprinting With CpG Island-Specific Primer Sets (MFSP)

MFSP focuses on the methylation status of the CpG-Islands and comprises three major steps. See FIG. 3.
Step 1: Genomic DNA is digested with a methylation sensitive or insensitive restriction enzyme.
Step 2: Digestion products from Step 1 are subject to a PCR amplification step. The PCR primers are selected such that a forward primer contains the methylation sensitive or insensitive restriction site. Consequently, all amplified PCR products comprise a nucleotide sequence matching the methylation sensitive or insensitive restriction site thereby indicating a methylation event. The reverse primer is selected such that the size of the PCR products are easily handled by the following experimental steps.
Step 3: PCR products from step 2 are resolved on one dimensional electrophoresis.

In one embodiment, MFSP comprises CpG-Island specific primer sets that are strongly biased towards CpG Island. (supra) These primer sets are selected and constructed in an identical manner as described above in the GMSP section. The results of one embodiment of using MFSP is shown in FIG. 8.

MFSP is not limited to the detection of methylation disease biomarkers but may also be useful to study global DNA methylation fingerprints. Like GMSP, MFSP also combines the utility of restriction endonucleases and PCR amplification. For example, a methylation sensitive enzyme distinguishes between a methylated and unmethylated restriction site (i.e., by cleaving only at the unmethylated restriction site). PCR primers, on the other hand, may be designed to selectively amplify part of the genome that was of interest. Further, a combination of a methylation sensitive restriction enzyme and the genome-wide specificity of CpG-Island specific PCR primers, provides a utility in creating an informative fingerprint representation of genomic methylation events. In alternative embodiment, MFSP can also scan for point mutations and deletions/insertions.

MSFP is believed advantageous in comparison to other methyl detection methods currently practiced as being simpler and better suited for automation and high-throughput applications. For example, the detected methylation signal is generated within the DNA fragment and not at the terminal ends (i.e., for example, as in RLGS), thereby reducing background interference. Another advantage of MFSP comprises flexibility and scalability. For example, by using a number of different primer pair sets, one can scan for DNA methylation events in the genome at different levels of “coverage” (i.e., nucleic acid sequence number “windows”).

In one embodiment, a nucleic acid window comprises a large number of nucleic acids (i.e., thereby generating a long primer) for use on a relatively small number of samples to screen for interesting and unique patterns. Although it is not necessary to understand the mechanism of an invention, it is believed that that this large nucleic acid window identifies a small number of primer pairs which are directed at the most interesting methylation patterns. Additional primer sets can be selected for amplification of tens, hundreds or even thousands of DNA fragments.

Some currently practiced techniques (i.e., for example, RLGS) detect signals generated from unmethylated sites, thereby requiring that DNA methylation is inferred by the absence of a fragment. In contrast, MFSP directly detects methylation because the marker is placed at the methylated restriction site. This advantage makes it is possible to find rare methylation events, and, for example, to detect DNA hypermethylation in a remote medium, such as blood or sputum, where the methylated DNA site is diluted by the presence of a much larger percentage of normal tissue. Another advantage of MFSP is that since the PCR products have primer-specific boundaries, their length can be predicted and a virtual electrophoresis image pattern can be generated. See FIG. 3.

Many variations of MFSP, in addition to those described above, can be made for specific purposes. For example, the CpG-Island specific primer sets can be made longer so the amplification will be more specific or the primer sets can be made to be degenerate so more fragments are detected. In another embodiment, a reverse primer also contains a methylation sensitive enzyme cutting sites thereby amplifying DNA fragments comprising at least two methylated cytosines (one from forward primer, the other from the reverse primer). Although it is not necessary to understand the mechanism of an invention, it is believed that this advantage would greatly simplify the methylation fingerprinting patterns.

MFSP PCR products may be coupled with alternative electrophoresis systems, including, but not limited to, two-dimensional gel system, that will increase resolution. Moreover, the PCR fragments generated from MFSP can also be used in microarray type of assays, in which PCR products of undigested DNA can be spotted on the array and hybridized by PCR products of digested DNA. In addition, MFSP methodology can be combined with some existing technologies such as AIMS. In the AIMS method, PCR amplification is difficult because at least more than 60% of the restriction site pairs are more than 2000 bp away from each other. Paz et al., Hum. Mol. Genet. 12:2209-2219 (2003). In one embodiment, the present invention contemplates an AIMS protocol modification using one primer matching an adapter linker and a second internal primer that matches a sequence between the two restriction sites. Thousands of 10mers are contemplated by the present invention that are within 2000 bp of the restriction site (i.e., for example, CCCGGG; SEQ ID NO:201) wherein each 10mer matches a significant fraction of the restriction fragments and many show a bias towards a CpG Island sequence.

### EXPERIMENTAL

The following examples are only intended as illustrative and are not intended to provide any limitations to the present invention.

### Example I

### Isolation of DNA from Tissue and Cell Lines to Measure Levels of Methylation

DNA was first isolated from normal and asthmatic lung tissue, normal and prostate cancer cell lines, and normal and lung cancer cell lines from stages I, II, IIIa, IIIb and IV. The lung tissue was pulverized using a Freezer Mill (Spex Certiprep—Catalog No. 6750) following the manufacturers recommendations. DNA from the pulverized tissue and the cell lines was isolated from using DNA isolation kits (Qiagen—Catalog No. 13343) following the manufacturers recommendations. Once the DNA was isolated the quantity the quality of the material was determined.

The quality and quantity of the DNA was measured on a UV spectrophotometer (Beckman DU 650 Spectrophotometer). The DNA was measured at two wavelengths (260 nm and 280 nm). The optical density (OD) at 260 nm wavelength determined the concentration of the DNA (OD at 260 nm×dilution×50) whereas the ratio of 260 m over 280 nm determined the purity of the DNA. If the ratio was ˜1.8 then the DNA purity was high and free of proteins and other contaminants.

The quality of the DNA was visualized by taking 200 ng of the sample and loading it onto a 1% agarose gel.

1. Measurement of Global Methylation in Lung Cancer Using GM-RESA

DNA isolated from normal and lung cancer (Stages I, II, IIIa, IIIb and IV) cell lines were analyzed using GM-RESA. The assay was performed in triplicate for each sample. Genomic DNA was aliquoted into each well of a 96 well PCR plate. For methyl sensitive restriction digestions with HpaII and BssHII (150 ngs, respectively) was aliquoted in triplicate. For methyl insensitive restriction digestions with MspI (100 ngs) was aliquoted in triplicate. The digestion with MspI was used to normalize the data from the HpaII and BssHII digestions. An equal amount of DNA was aliquoted for incubation in buffer only, which would serve as a control for background.

To prevent any down stream reactions occurring at 5′ or 3′ overhangs of the genomic DNA, which would have occurred due to shearing in the DNA isolation step, the genomic DNA was end-filled with dideoxynucleotides using Sequenase Version 2.0 T7 DNA Polymerase (USB—Catalog No. 70775Z). The reaction was performed in a total volume of 20 μl and contains 1× Sequenase® buffer, 1 unit Sequenase®, and 0.4 μM each of dideoxy (ATP, CTP, GTP, and TTP) The reaction was left at 37° C. for 20 minutes and terminated by incubation at 75° C. for 10 minutes.

The DNA was cleaned up using CleanSEQ dye-terminator removal magnetic beads (Agencourt Catalog No. 000121) according to the manufacturers instructions After this step the DNA was then digested with methyl sensitive (HpaII and BssHII) and insensitive (MspI) enzymes. The reaction was performed in a total volume of 45 μl containing 1× of the appropriate buffer (New England Biolabs) and 1 U of restriction enzyme. The reaction was left at the appropriate temperature for the enzyme for 2 hours.

This was then followed by an end-fill reaction with biotin labeled dCTP and dGTP (Perkin Elmer—catalog number Nel 538001EA and Nel 541001EA) using Sequenase®. The reaction was performed in a total volume of 75 μl and contains 1× Sequenase® buffer, 1 unit of Sequenase®, and 0.1 uM of biotin labeled dCTP and dGTP. The reaction was left at 37° C. for 20 minutes and terminated by incubation at 75° C. for 10 minutes. Reacti-Bind DNA Coating Solution (1001: Pierce; Catalog No. 17250) was mixed with the biotin end-labeled DNA and the solution was transferred to a 96 well white Microfluor 2 plate (Thermo Electron Corp—Catalog No. 7905). The plate was left on a shaker for 18 hours at room temperature. The solution was removed after the 18 hour incubation and the wells were washed with TBS (10 mM Tris-HCL, pH 8, 150 mM NaCl) three times, 2 minutes for each wash.

Biotin was detected using the DNADetector HRP Chemiluminescent Blotting Kit (KPL Catalog No. 54-30-00) with the following modifications. After the final wash, 200 μl per well Detector Block was added and incubated for 30 minutes at room temperature to block the wells. The blocking solution was aspirated and replaced with 175 μl Detector Block containing 1:2000 HRP Neutravidin (Pierce Catalog No. 31001). The Neutravidin mix was aspirated and the wells washed four times for five minutes with Biotin Wash. LumiGLO (175 μl/well) was added and after 2 minutes the luminescence was read on a Wallac Envision 2100 multilabel reader (Perkin Elmer) using a luminescence dual BRET 2 mirror and a luminescence 700 emission filter. The data was processed in Microsoft Excel.

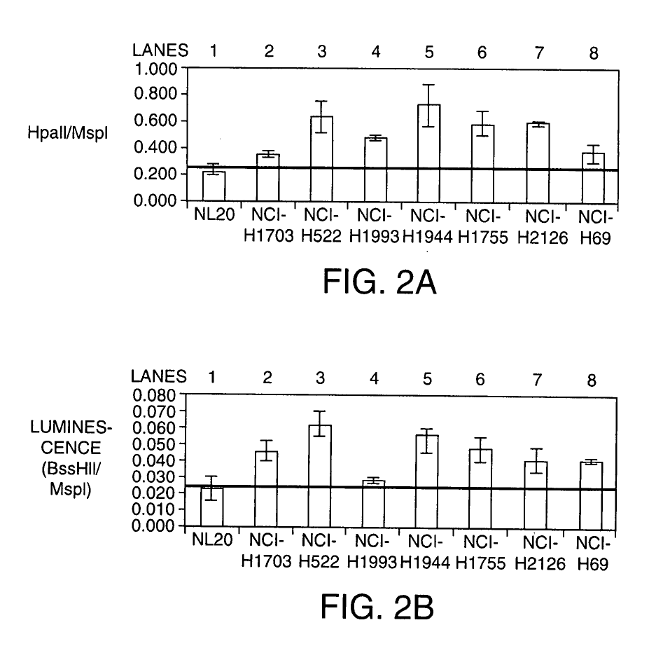

The results showed that with the HpaII digestion there was a large amount of hypomethylation occurring in the lung cancer cell lines, which was above the normal lung cell line. See FIG. 2. In general the hypomethylation increased as the cancer stage increased. With the BssHII, the results indicated that again there was more hypomethylation in the cancer cell lines than hypermethylation. This was not evident in the prostate cancer cell line, which showed more hypermethylation and hence more BssHII were methylated and thus the enzyme was unable to cut at these sites. These data indicate that in lung cancer there is a large amount of genomic instability, which causes a higher frequency of hypomethylation. This phenomenon can be used as an early diagnostic for lung cancer, as well as measuring prognosis and the efficacy of a therapeutic treatment toward curing the disease.

### Example II

### Methylation Fingerprinting with a Pair of CpG Island Specific Primers

This example presents one embodiment of the present invention comprising one CpG Island specific primer pair (Forward primer: GTCTCGTGGT; SEQ ID NO:202; Reverse Primer: AGGTACCGGG; SEQ ID NO: 203) to demonstrate the methylation fingerprinting. See FIG. 8. The reverse primer comprises a methylation insensitive enzyme MspI restriction site (CCGG; SEQ ID NO:204) and the forward primer comprises a restriction site for the methylation sensitive isoschizomer HpaII.

Human genomic DNA (Novagen) was digested with HpaII (lane 1) or MspI (lane 3, New England Biolabs) at 37° C. overnight. Control DNA was incubated in the corresponding digestion buffer without enzyme (lane 2 and lane 4). PCR was carried out in 1× GC Buffer (Finnzymes), 400 uM dNTPs, 5% DMSO, 0.02 U/μl Phusion DNA polymerase, 0.4 ng/μl DNA template, and 5 μM primers. An initial denaturation at 98° C. for 30 seconds was followed by 40 cycles of 98° C. for 10 seconds, 48° C. for 60 seconds and 72° C. for 60 seconds with a final extension of 72° C. for 5 minutes. PCR products were resolved on a 1.6% agarose gel and visualized with ethidium bromide. The controls that were incubated with HpaII and MspI buffer gave rise to very similar fingerprints (lane 2 & 4). The fact that many bands in lane 4 either disappeared or showed decreased intensity in lane 3 confirmed that these PCR amplification products do contain the restriction site CCGG. Those bands that disappeared from MspI digestion, but remained in HpaII digestion suggested methylation events in the amplified regions. See FIG. 8—asterisks. Those bands that disappeared from both MspI and HpaII digestions suggested unmethylated CG sites, which are more likely to reside within promoter regions of protein encoding genes in normal genomic DNA (arrows in FIG. 8).

### Example III

### Identification of Novel DNA Methylation Biomarkers in Asthma using the Methylation Sensitive Amplification System (MESAS)

DNA isolated from normal and asthma lung tissue was analyzed using MESAS. For each sample 2 μg of genomic DNA was aliquoted into an Eppendorf® tube. To prevent any down stream reactions occurring at 5′ or 3′ overhangs of the genomic DNA, which may have occurred due to shearing in the DNA isolation step, the genomic DNA was end-filled with dideoxynucleotides using Klenow (exo-) (NEBioLabs—M0212L). The reaction was performed in a total volume of 35 μl and contains: 2 μg genomic DNA in 25 μl water, 9 μl blocking buffer and 1 μl (5 U) Klenow (exo-) DNA Polymerase. The reaction was left at 37° C. for 30 minutes and terminated by addition of 1/10 volume (3.5 μl) of 100 mM EDTA and incubated at 80° C. for 30 min.

The DNA was cleaned using AutoSeq G50 spin columns (Amersham-27-5340-02). After this step the DNA was then digested with a methyl specific enzyme BisI which will cuts the DNA at positions that are methylated. The reaction was performed in a total volume of 46 μl and contains 2 μg genomic DNA in 34 μl of water, 8 U of BisI, and 4 μl of enzyme buffer. The reaction was left at 37° C. overnight (18 hrs) and terminated by buffer removal using AutoSeq G-50 spin column.

The DNA digest was end-filled using a mixture of all 4 dideoxynucleotides (Roche—PCR Nucleotide Mix—1 581 295) using DNA Polymerase I Large (Klenow) Fragment (NEBioLabs—M0210L). The reaction was performed in a total volume 25 μl containing 1.8 μg genomic DNA in 20 μl of water, 2.5 μl NE Buffer 2 (NEBioLabs—B7002S), 0.84 μl 1 mM stock of deoxynucleotides (final concentration 33 μM), 1.26 μl water, 0.35 μl (1.8 U) of DNA Polymerase I. The reaction was left at 25° C. for 15 min and terminated by the addition of 2.5 μl 100 mM EDTA and heat deactivated at 80° C. for 30 min followed by a cleaning step with AutoSeq spin column and volume was reduced to 15 μl using an Automatic Environmental SpeedVac System (Savant).

The end-filled DNA was ligated to a double stranded oligonucleotide linker (oligo1/oligo2), which contains a unique sequence that is not homologous to any DNA sequence within the human genome. The linker also contained an EcoRI restriction site to clone fragments into an EcoRI linearized vector. Each single stranded oligonucleotide linker (10 μg), which were complementary to each other, were annealed by heating them together in an Eppendorf® tube to 100° C. for five minutes. The tube was cooled to 37° C. over 30 minutes in a thermocycler using a ramping step of 0.9° C. per every 30 sec. The double stranded oligonucleotide linker (1 μg) was ligated to the end-filled DNA using 400 U of T4 DNA ligase (NEBioLabs—M0202L). The reaction was performed in a total volume of 30 μl and contains 1.8 μg genomic DNA in 20 μl of water, 10 μl of annealed oligo 1 and oligo 2 (1 μg each), 3 μl 10×T4 DNA Ligase buffer, 1.5 μl 5 mM dATP and 1 μl of T4 DNA Ligase. The reaction was left overnight (18 hrs) at 15° C. The DNA was cleaned using an AutoSeq spin column to remove any excess double stranded oligonucleotides. One microliter, 2 μl and 5 μl of the ligated product was PCR amplified to optimize the amount of material that will generate robust bands. Each PCR reaction contains a PCR 5′ primer that is complementary to oligo 2 of the linker and an additional sequence at the 3′ end that is complementary to a methylation sensitive or methylation insensitive restriction site followed by two degenerate bases.

For each PCR reaction the following is added:

1.0 μl/0.5 μl/0.1 μl ligated product

10 μl Finnzymes Phusion Master Mix GC® (NEBioLabs—F532S)

10 μl PCR primer (10 μM stock) (final concentration 0.5 μM)

0.6 μl DMSO

Water added to a final volume of 20 μl.

The PCR conditions were as follows:

Step 1: Denature—98° C./30 sec

Step 2: Denature—98° C./10 secs

Step 3: Anneal—55° C./30 sec

Step 4: Extension—72° C./2 min

Step 5: Repeat Steps 2 through 4; ten times

Step 6: Denature—98° C./10 secs

Step 7: Extension—72° C./2 min

Step 8: Repeat Steps 6 through 7; 25 times

Step 9: Long extension—72° C.

Step 7: 4° C. hold

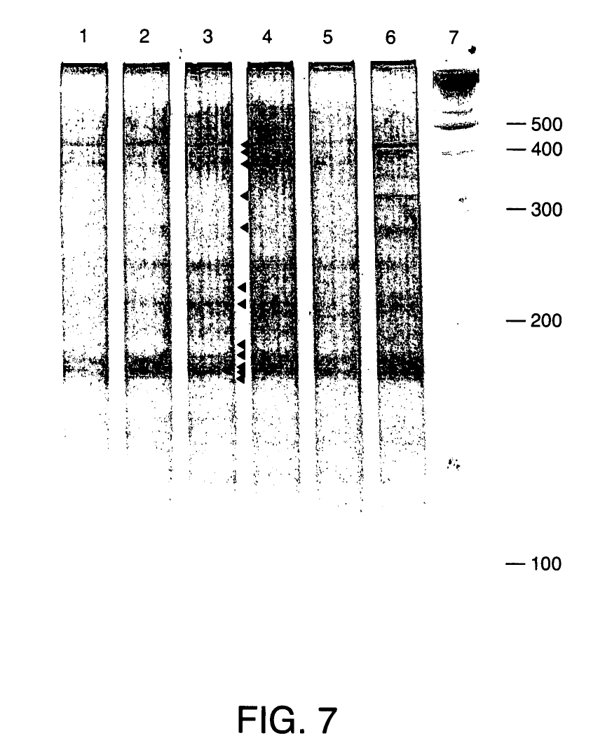

The PCR products were separated using 4% to 20% gradient polyacrylamide gel electrophoresis (Bio-Rad—345-0060) using 1×TBE buffer for 4 hours at 130 volts. Differences in band intensity or presence or absence of bands were quantitatively scored. See FIG. 7. The fragments were cut out of the gel, crushed and the DNA eluted using elution buffer (0.5 M ammonium acetate and 10 mM magnesium chloride). The DNA was ethanol precipitated and cloned into a PCR4 Blunt-TOPO vector using the Zero Blunt TOPO PCR cloning kit (Invitrogen) for propagation into an E. coli host using standard molecular biology techniques. The cloned fragments will be sequenced (Agencourt) and the sequence will be compared against the GenBank database by BLAST analysis to identify the location within the human genome that the fragments originate from.

### Example IV

### Experimental Conditions to Perform the Global DNA Methylation Assay

1. Optimization of Biotinylated Nucleotide Concentration in End-Fill Reactions

This experiment used various amounts of biotinylated nucleotides to determine the optimal concentration to maximize signal sensitivity.

The HpaII methyl sensitive restriction enzyme, which has 2.2×10⁶ restriction sites within the human genome, was applied to commercially available “normal male” genomic DNA (Novagen) to create a DNA digested products. An end-fill reaction of the digested products was performed following standard molecular biology procedures. In: Molecular Cloning A Laboratory Manual, Second Edition, Eds. J. Sambrook, E F Fritsch and T Maniatis; (1989). However, an exact adherence to the Maniatis procedures (as well as Novagen's recommendations) for the use and amount of biotinylated nucleotides was identifies as an unnecessarily expensive assay. For instance, the recommended protocols specified 33 μM nucleotides per end fill reaction. This was equivalent to ˜1.2×10¹⁵ molecules per reaction. However, the number of HpaII sites in 100 ng of genomic DNA that can be end-labeled is ˜6.6×10¹⁰ (i.e., for example, 2.2×10⁶ HpaII sites per genome multiplied by 30,000 genome equivalents in 100 ng of digested DNA). Thus, the biotinylated deoxynucleotides were in an inordinant and unnecessary excess over the amount of HpaII ends that would require end-filling/labeling.

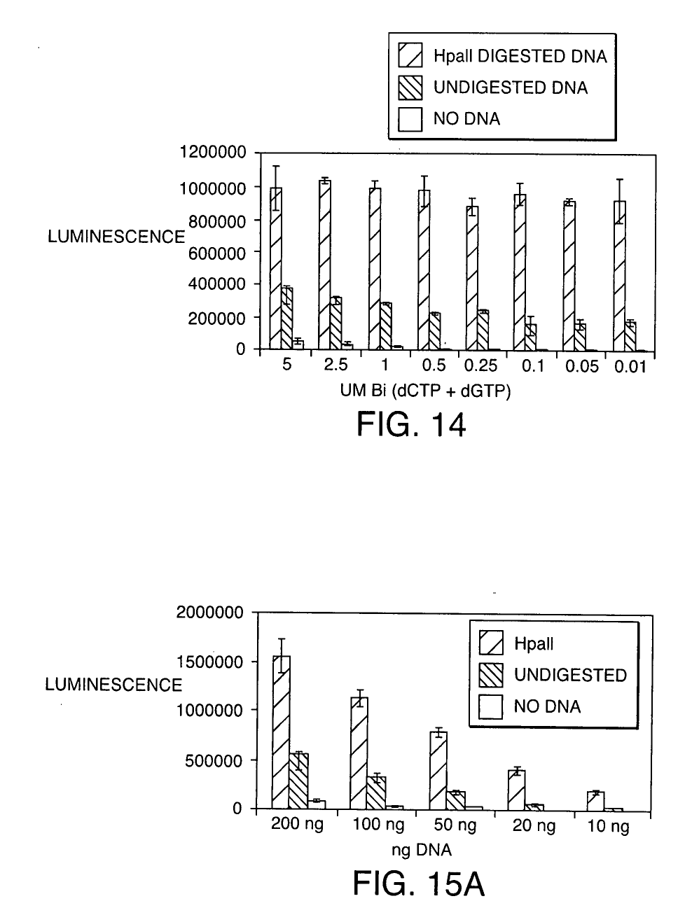

A determination of more practical levels of biotinylated nucleotides that could be applied in an end-fill reaction was designed. One hundred nanograms of genomic DNA was aliquoted in triplicate into a 96 well micro-titer plate and digested with 10 units of the methyl sensitive HpaII (CCGG) restriction enzyme, overnight at 37° C. HpaII leaves a 5′ CG overhang, therefore the only biotinylated deoxynucleotides that needed to be present in the end-fill reaction were biotin-dCTP and biotin-dGTP. The amount of biotin-dCTP and biotin-dGTP (Perkin Elmer) was titrated, starting at 5 μM (i.e., for example, 1.5×10¹⁴ biotinylated molecules per sample) down to 0.01 μM (i.e., for example, 3×10¹¹ biotinylated molecules per sample) and used 5 units of Exonuclease (−) Klenow DNA polymerase (NEB) in each of the end-labeling reactions. The DNA was transferred to a white 96 well Microfluor® 2 plate (Thermo Electron) and mixed with Reacti-Bind® (Pierce) to adhere the DNA to the surface. The biotin was detected using the HRP Chemiluminescence kit (HRP) and quantitated by a Wallac Envision 2100 multilabel reader (Perkin Elmer). The results indicated that a high amount of luminescence was detected even when using as little as 0.01 μM biotinylated nucleotides (FIG. 14). These data suggested that the use of biotinylated nucleotides at the level recommended by standard molecular biology protocols as well as the manufacturer was far in excess of what was necessary for the for this type of reaction and this assay. We chose to use 0.5 μM biotinylated deoxynucleotides for the experiments presented herein.

2. Optimization of Signal/Noise Ratio Using Streptavidin & Neutravidin

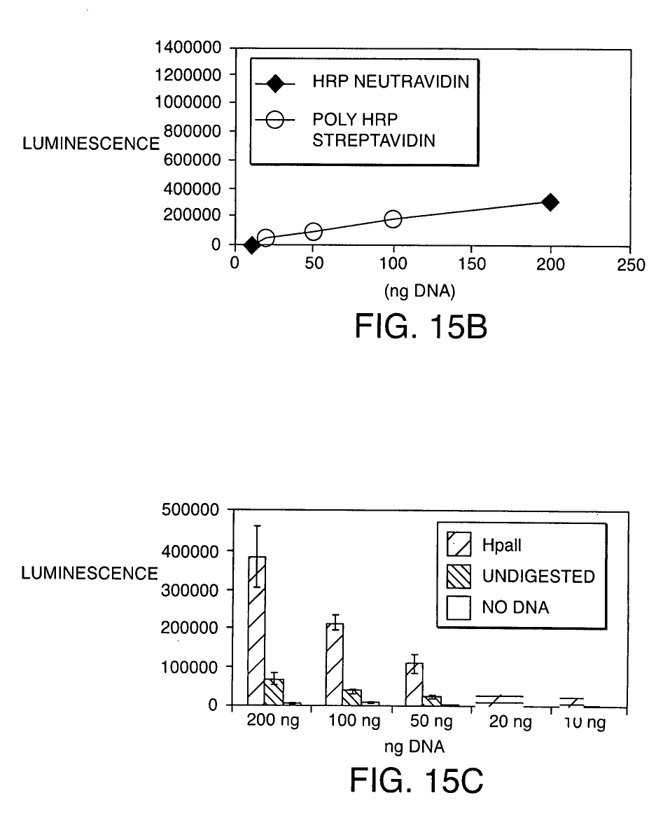

In order to increase the signal to noise ratio, streptavidin was compared with neutravidin. Streptavidin was used in the HRP Chemiluminscence Kit (KPL) to detect and quantitate the amount of biotin that was incorporated in the end-fill reaction. However, neutravidin has a lower non-specific binding to most sugars when compared to other biotin binding proteins due to the lack of a carbohydrate and a neutral pH solution. To compare the two avidins, five sets of 1 μg genomic DNA (Novagen) was aliquoted in triplicate in a 96 well micro-titer plate, digested with 10 units of HpaII over-night at 37° C. and end-labeled with 5 units Exonuclease (−) Klenow DNA polymerase (NEB) using 0.5 μM biotin-dCTP and biotin-dGTP. Two hundred nanograms of DNA was aliquoted in triplicate from the first set of digestions and transferred to a white 96 well Microfluor® 2 plate (Thermo Electron), 100 ng, 50 ng, 20 ng and 10 ng was aliquoted from the digestions of the second, third, fourth, and fifth set respectively. A duplicate plate of the titrated aliquots was made. Both plates were treated with Reacti-Bind® (Pierce) to adhere the DNA to the surface of the plate. Streptavidin was added to one plate and neutravidin to the other and the HRP Chemiluminescence Kit (KPL) was used to detect the biotin. Quantitation was by a Wallac Envision 2100® multilabel reader (Perkin Elmer). The results indicated that when using neutravidin the signal was 4 times less than with streptavidin. However, the signal to background ratio improved two-fold when using neutravidin (FIG. 15A). A comparison of the linear range of the assay using streptavidin and neutravidin indicated that the former avidin protein had a linear range between 10-20 ng and the latter between 10-200 ng (FIG. 15B). Thus neutravidin gave a better signal to noise ratio with a broader linear range of DNA than streptavidin.

3. Optimization of Biotinylated Nucleotides Incorporation in End-Fill Reactions

The signal over the background noise was addressed by optimizing the end-fill reaction. The procedure was modified to fit within the parameters of the assay, mostly directed to low cost and ease of use. The end-fill reaction was evaluated to identify the steps where variables could be applied and measured such that when the procedure would be re-constituted, it would develop a streamlined method.

Three steps in the end-fill reaction were identified where variables could be applied. The efficacy of the procedure to incorporate biotinylated nucleotides to a 5′ overhang was measured under the various conditions:

Incubation time (at 37° C.): 10, 20 and 30 minutes;

Amount of biotin-dCTP and biotin-dGTP: 1.0 μM, 0.5 μM and 0.1 μM; and

Amount of Klenow (Exonuclease (−) Klenow DNA polymerase (NEB)):

5 units, 2.5 units and 1 unit.

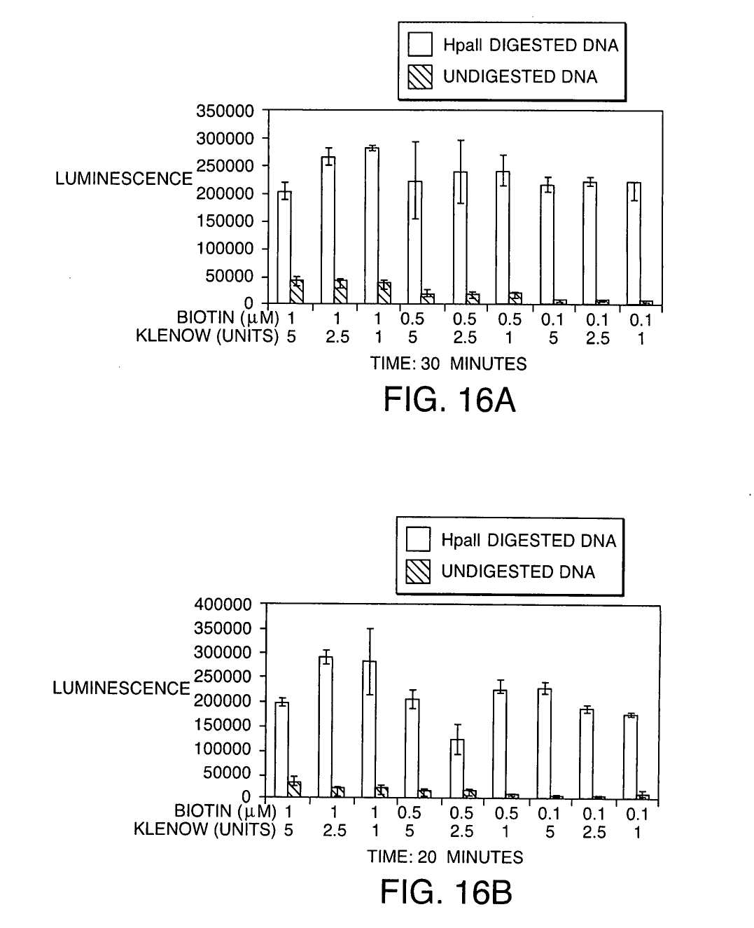

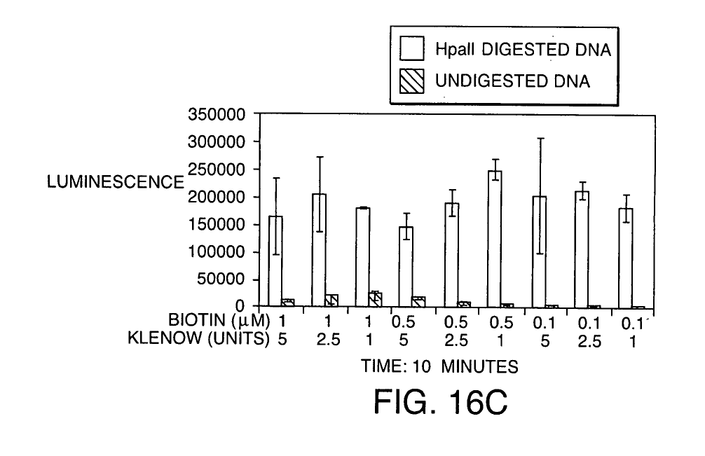

One hundred nanograms genomic DNA (Novagen) was aliquoted in triplicate per data point and digested with 10 units HpaII over-night at 37° C. Many combinations of time and amounts of biotinylated nucleotides and Klenow were used in the end-fill reactions. The end-filled DNAs were transferred to a white 96 well Microfluor® 2 plate (Thermo Electron) and mixed with Reacti-Bind® (Pierce) to adhere the DNA to the surface. The biotin was detected using neutravidin and the HRP Chemiluminescence kit (HRP) and quantitated by a Wallac Envision 2100 multilabel reader (Perkin Elmer). The results indicated that a combination of 0.1 μM biotin-dCTP and biotin-dGTP, 2.5 units Klenow, incubated for 10 minutes at 37° C. gave the best signal to background (FIG. 16).

4. Optimization of DNA Polymerase in End-Fill Reactions

The manufacturer's data sheet for Exonuclease (−) Klenow DNA polymerase indicated that the enzyme possesses a small amount of endonuclease activity, which could introduce a higher background. This enzyme was compared with other DNA polymerases that identified Sequenase® as a possible substitute. Sequenase® (USB) is made from T7 DNA polymerase, has virtually no 3′ to 5′ exonuclease activity and is highly processive. This enzyme was used in a similar experiment to the one described above and the incubation time and amount of biotinylated deoxynucleotides was varied. A fixed amount of Sequenase® as recommended by the manufacturer was used in this experiment.

Incubation time (at 37° C.): 10, 20 and 30 minutes;

Amount of biotin-dCTP and biotin-dGTP: 0.1 μM, 0.01 μM and 0.001 μM; and

Amount of Sequenase®: 1.0 unit

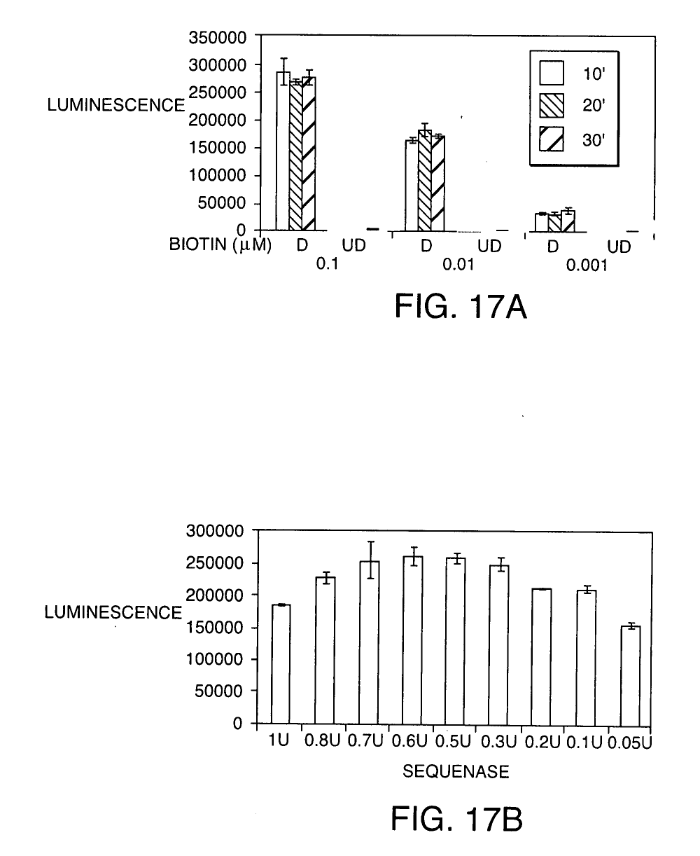

The results indicated that Sequenase® produced a background that was even lower than the Exonuclease (−) Klenow DNA polymerase but with an equivalent amount of signal (FIG. 17A). Optimal conditions were observed when using 0.1 μM biotinylated nucleotides. To determine further the value of Sequenase® to incorporate the biotinylated nucleotides, Sequenase® was titrated in a similar experiment as described above. The incubation time and amount of biotinylated nucleotides was fixed and the Sequenase® was titrated from 1.0 unit to 0.05 units.

Incubation time (at 37° C.): 30 minutes;

Amount of biotin-dCTP and biotin-dGTP: 0.1 μM; and

Amount of Sequenase: 1.0 unit, 0.8 units, 0.7 units, 0.6 units, 0.5 units, 0.3 units, 0.2 units, 0.1 units and 0.05 units.

The results indicated that the amount of Sequenase® added into each experiment produced similar methylation indices when using a high amount (i.e., for example, 1.0 unit as recommended by the manufacturer) or a low amount (i.e., for example, 0.05 units) (FIG. 17B). Consequently, 0.1 unit of Sequenase® was chosen as the optimal amount for most end-labeling reactions.

5. Linearity of End-Labeling Reaction

In order to ensure that the above optimized set of end-labeling conditions was linear with respect to the amount of DNA ends available in the reaction, an experiment was performed using MspI to digest the DNA that measured the incorporation of biotinylated nucleotides after digestion. MspI is a methyl insensitive restriction enzyme and an isoschizomer of HpaII (restriction site: 5′-C/CGG-3′). Although it is not necessary to understand the mechanism of an invention, it is believed that since there are approximately 2.2×10⁶ sites available within the human genome, this enzyme would represent an excellent means to measure the efficacy of the new end-labeling conditions. Incorporation of the biotinylated nucleotides into the 5′CG overhang of the MspI digested DNA was exected to be proportional to the amount of DNA being assayed.

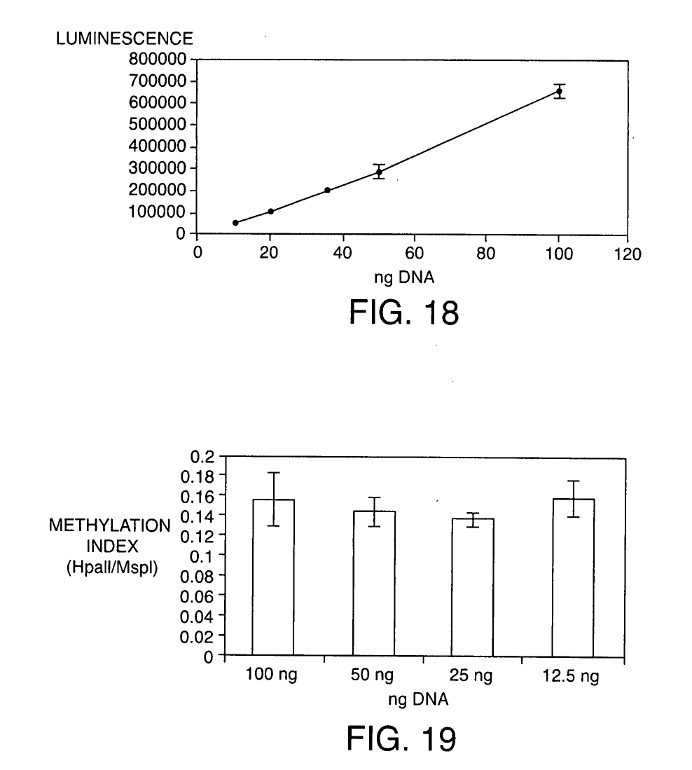

To determine if there was a linear relationship between the amount of DNA digested and the end-fill reaction using biotinylated nucleotides, 1 μg of genomic DNA was aliquoted in triplicate into a 96 well plate and digested with 10 units of MspI, overnight at 37° C. The DNA was end-labeled with 1 unit of Sequenase® and 0.1 μM biotin-dCTP and biotin-dGTP. One hundred nanograms, 50 ng, 20 ng, and 10 ng of DNA was aliquoted from each of the MspI digested reactions and transferred to a white 96 well Microfluor® 2 plate (Thermo Electron). The DNA was mixed with Reacti-Bind® (Pierce) to adhere it to the surface of the plate. The biotin was detected using neutravidin and the HRP Chemiluminescence kit (HRP) and quantitated by a Wallac Envision 2100 multilabel reader (Perkin Elmer). The results indicated that there was a linear relationship between the amount of DNA digested and the amount of biotin incorporated in an end-fill reaction, which ranged from 10 ng to 100 ng (FIG. 18).

6. Normalization of Methylation Sensitive Restriction Enzyme Reactions

The inter-sample variation of the genomic DNA concentration can cause problems when performing comparative analysis, no matter how careful the process of quantitation. This is particularly important when comparing methylation in normal versus affected DNA samples. Problems may occur if the DNA is not uniformly in solution, which can be compounded by the pipetting errors that may occur in aliquoting the material. A normalization step would abrogate these problems. The restriction enzyme MspI is methyl insensitive and an isoschizomer of HpaII (CCGG) and in effect can be used to normalize the digests of methylation sensitive restriction enzymes.

To determine if the application of a normalization step will correct for errors in DNA concentrations 1 μg of genomic DNA was aliquoted (in triplicate) into a 96 well micro-titer plate and digested it with 10 units of HpaII, over-night at 37° C. In another 96 well micro-titer plate the same amount of DNA was aliquoted (in triplicate) and digested with 10 units of MspI, over-night at 37° C. The DNA was end-labeled with 1 unit of Sequenase® and 0.1 μM biotin-dCTP and biotin-dGTP. One hundred nanograms, 50 ng, 25 ng and 12.5 ng of DNA was aliquoted from each of the HpaII and MspI digested reactions and transferred to a white 96 well Microfluor® 2 plate (Thermo Electron). The biotin was detected using neutravidin and the HRP Chemiluminescence kit (HRP) and quantitated by a Wallac Envision 2100 multilabel reader (Perkin Elmer). The chemiluminescence values from the HpaII digestions were divided by the MspI values to generate a normalized HpaII result (FIG. 19). The results indicated that there was a good level of normalization of the HpaII chemiluminescence values across broad ranges of DNA concentrations. These data indicate that a normalization step using MspI would be of value in correcting for DNA concentration errors for HpaI digestions and can be applied in combination with other methyl sensitive restriction enzymes.

7. Optimization of DNA Sample Size for GM-RESA

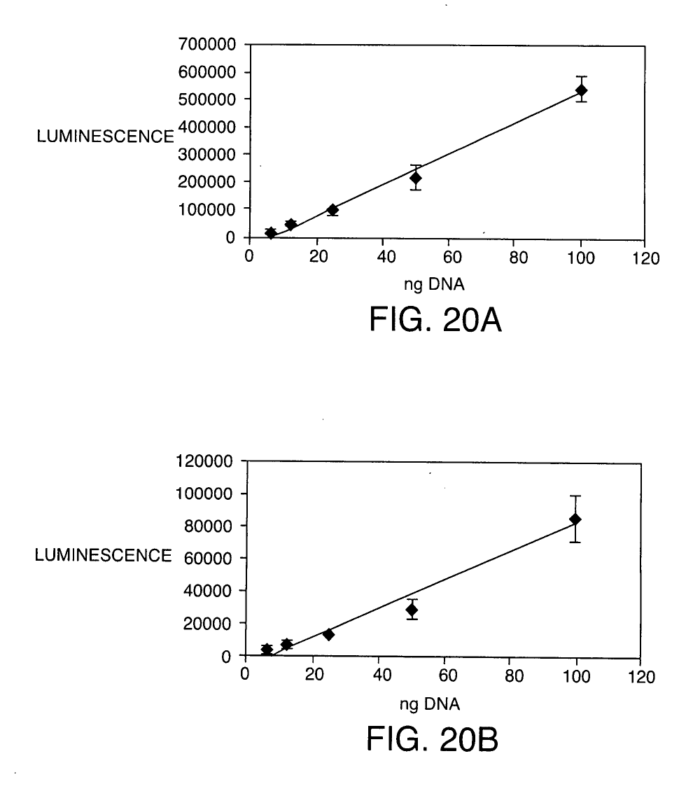

A non-invasive assay may be performed by acquiring DNA from either whole blood or buccal cells collected from buccal washings using mouthwash or water, or alternatively by scraping the inner cheek. However, the amount of remote tissue needed will be determined by the assay's ability to detect the changes in DNA methylation. A titration curve was performed to measure the lowest amount of DNA that the assay can detect when using the two enzymes HpaII and MspI. The chemiluminescence values were plotted against the amount of DNA. A linear curve was observed for both enzymes that ranged from 12.5 ng to 100 ng for the HpaII and MspI digests (FIGS. 20A and 20B). The minimum amount of DNA observed to successfully perform a HpaII and MspI digest was 50 ng; midway on the linear curve. Thus, the minimal amount of DNA to measure global methylation per clinical sample was calculated as 1 μg of DNA (3×50 ng for the MspI and 3×50 ng HpaII digests, and 3×50 ng for MspI and HpaII controls) was sufficient to repeat the experiment twice.

8. Comparison of GM-RESA to HPCE

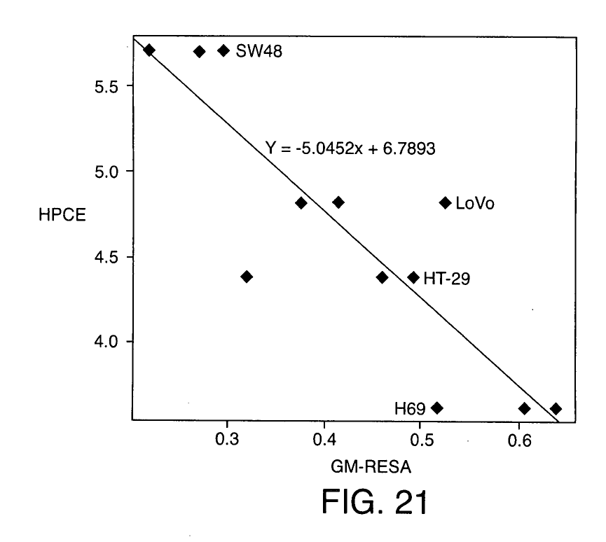

High-performance chromatography electrophoresis (HPCE) is generally viewed as the “gold standard” in measuring the global genomic content of 5-methylcytosine. GM-RESA, therefore, was compared with HPCE to measure global DNA methylation in four lung cancer cell lines (i.e., for example, SW48, LoVo, HT-29 and H69; ATCC). These cell lines varied in the content of the global DNA methylation from high to low as determined by HPCE. Paz et al. Cancer Res 63:1114-1121 (2003). The four cell lines were grown according to the data sheets and the DNA was isolated using the Blood and Cell Culture DNA Midi Kit (Qiagen). The amount of methylation in the genomes of the four cell lines was measured using HpaII digestion normalized against MspI using the optimized conditions outlined above. Triplicate data points from the GM-RESA were used in a comparison analysis to determine whether a strong correlation existed between the two technologies. The GM-RESA results gave a good linear fit compared to the HPCE data, suggesting that the number of HpaII sites has an inverse linear relationship with the total number of methylated cytosines (FIG. 21). Residual standard error of regression was 0.4094, which is about 10% of the measured results. These results represented a useful range. The linear fit of this correlation can be represented as HPCE=−5.0452×GM-RESA+6.7893. This result confirmed that GM-RESA could be utilized as a simple, time and cost effective approach to assessing the global DNA methylation level in the genome.

9. Analytical Sensitivity of GM-RESA

The analytical sensitivity of a GM-RESA assay may be defined as the probability that a test will detect an analyte, a mutation, or an alteration within a specimen. In this case, the analytical sensitivity of GM-RESA represents the lowest changes in methylation that is distinguishable from background noise.

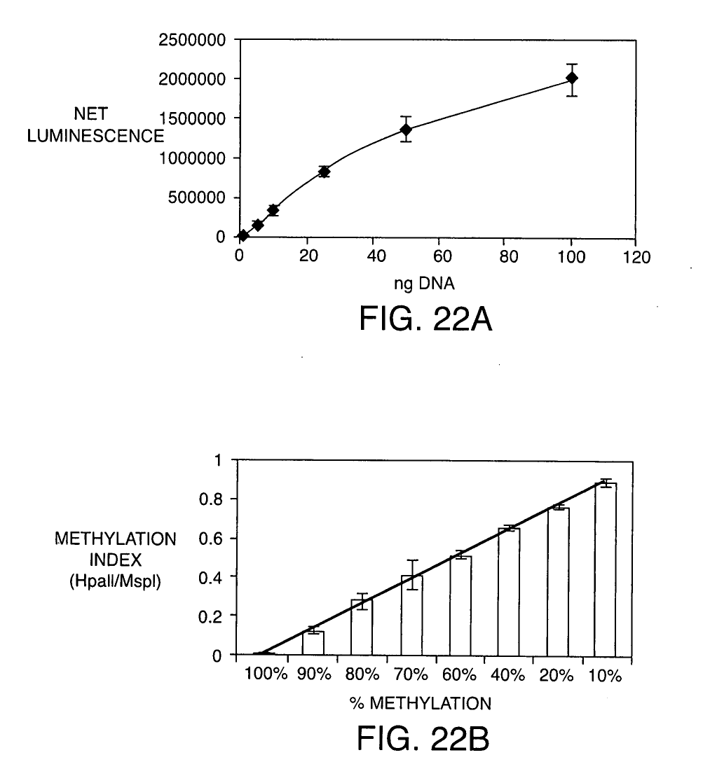

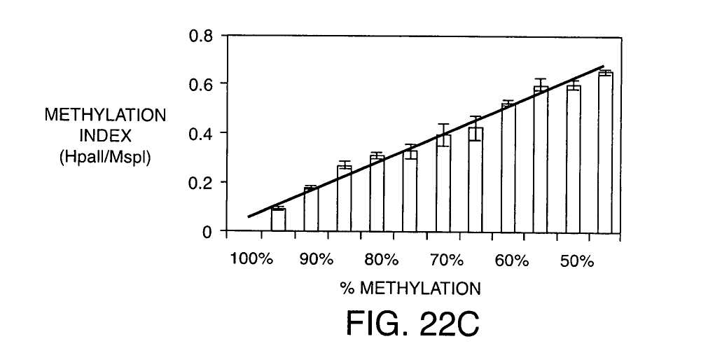

Lambda DNA was chosen as the test DNA to measure the analytical sensitivity of GM-RESA in the 96 well plate. The linearity of the assay was tested by measuring the amount of chemiluminescence emitted per concentration of Lambda DNA. Here Lambda DNA of varying concentrations (i.e., for example, 0.1 ng, 0.5 ng, 1.0 ng, 5.0 ng, 10.0 ng, 25.0 ng, 50 ng, and 100 ng) was placed, in triplicate, into 96 well Microfluor® 2 White plate and in each well 10 units of MspI was added. The DNA was digested for 2 hours at 37° C. The end-fill reaction was performed using 0.1 unit Sequenase® and 0.1 μM biotin dCTP and dGTP for 30 minutes at 37° C. The biotin was detected using neutravidin and the HRP Chemiluminescence kit (HRP) and quantitated by a Wallac Envision 2100 multilabel reader (Perkin Elmer). A graph was plotted of luminescence versus DNA concentration (FIG. 22A). The curve was found to be linear up to 25 ng and this amount of DNA was chosen to measure the analytical sensitivity of GM-RESA. To determine the analytical sensitivity of GM-RESA the entire genome of Lambda DNA was methylated using SssI, a bacterial enzyme that methylates cytosine residues within a CpG dyad. The methylated Lambda DNA was mixed with unmethylated Lambda DNA so that the percent of methylated DNA (by mixing with unmethylated DNA) increased in increments of 10%, spanning a range from 10% to 100%. Twenty five nanograms of each DNA mix was placed directly into a Microfluor® 2 White plate, in triplicate, and digested with the methyl sensitive enzyme HpaII and in another aliquot the DNA was digested with the methyl insensitive enzyme MspI. After end-labeling with biotinylated nucleotides and measurement of the chemiluminescence, a graph was plotted of luminescence (HpaII/MspI) versus percent methylation (FIG. 22B). The results showed a linear 10% decrease in methylation of the Lambda DNA from 100% to 10% indicating that the analytical sensitivity under these experimental conditions was 10%. To refine the level of analytical sensitivity, the assay was repeated except that the increments of percent methylation (mixed with unmethylated) DNA increased every 5% from 100% to 50% (FIG. 22C). The results showed a linear 5% decrease in methylation of the Lambda DNA from 100% to 50% and that the analytical sensitivity was ˜5%, which was a vast improvement on the radiolabeled cytosine extension assay (supra). These data showed that GM-RESA was highly sensitive at detecting changes in global DNA methylation perturbations.

In addition to determining the analytical sensitivity, an entire GM-RESA assay was performed in a 96 well Microfluor® 2 White plate without any need to transfer from a first 96 well microtiter plate (where the digestion of the genomic DNA and the end-fill reaction would be performed) to a second 96 well microtiter plate (where the chemiluminescence reaction would be performed). This step has streamlined GM-RESA and further simplifies the process. Optimized GM-RESA assays, as outlined herein, were performed in a single 96 well Microfluor® 2 White plate.

### Example V

### Methylation Status of Lung Cancer Cell Lines

The optimized GM-RESA assay in accordance with Example VI was applied to the examination of a number of cell lines that represented the different stages of the Non Small Cell Lung Cancer (NSCLC) form of the disease.

Cell lines were purchased from ATCC and represented the full extent of the disease: Normal lung, Stage I, Stage II, Stage IIIa, Stage IIIb and Stage IV metastatic liver. The cell lines were grown according to the data sheets and the DNA was isolated using the Blood and Cell Culture DNA Midi Kit (Qiagen). The concentration of the DNA was measured using a UV spectrophotometer. The quality of the DNA was determined by loading 200 ng on a 1% agarose gel to inspect for any degradation. All DNA preparations were determined to be of high quality. The DNA was digested in triplicate with HpaII and MspI and end-labeled with biotin dCTP and dGTP using Sequenase® under optimal conditions in accordance with Example IV. The HpaII chemiluminescence values were normalized against those produced by the MspI digests to produce a global methylation index.

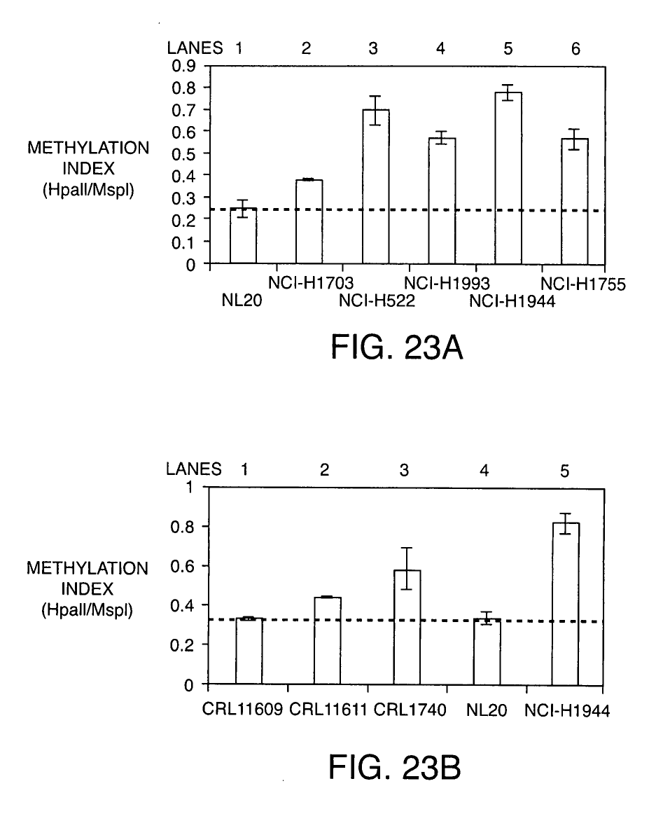

The results with HpaIl demonstrated that in all the stages of lung cancer as captured in the cell lines showed a higher methylation index than the normal (FIG. 23A). The methylation index in the normal cell line was a mean of 0.3±0.03 SD. The percentage methylation in the normal cell line was calculated:
(1−Methylation Index)×100%
1−0.3×100%=70%.

This is the exact amount of methylation that has been calculated to exist in a normal genome. Conversely using the above equation, the cancer cell lines showed a loss of methylation in their genomes compared with the normal level, which varied from 60% (Stage I) to 30% (Stage IIIb). This reproducible result indicated that the genomes of the cancer cell lines showed an increase in hypomethylation as the disease progressed from early (Stage I) to late (Stage IV) stage. Hypomethylation has been linked to activation of genes and genome instability in cancers, which in lung cancer is particularly acute. These data agree with many observations made in lung cancer and demonstrate that the level of hypomethylation is very high and can be accurately quantitated using GM-RESA.

To determine whether GM-RESA could detect changes in global DNA, methylation in other cell lines was determined using a normal, mild hyperplasia and a prostate cancer cell line (LNCaP) and compared with normal and Stage IIIb lung cancer cell line. The LNCaP cell line has been shown to have a high hypermethylation profile (i.e., for example, due to CpG island hypermethylation) as determined by Methylation Specific PCR (MSP). Paz et al, Cancer Res., 63(5):1114-1121(2003). The results with HpaII showed that there was global DNA hypomethylation in the LNCaP but not as much as the lung cancer cell line Stage IIIb (FIG. 23B). Although it is not necessary to understand the mechanism of an invention, it is believed that this was probably due to an excess regional hypermethylation that has been observed in this cell line. The mild hyperplasia cell line showed a significantly higher methylation index (0.42±0.01 SD) than the normal (0.34±0.01 SD) but was lower that the prostate cancer cell line (0.6±0.08 SD). This would suggest that the methylation status of the genome had reverted to a higher level, tending to a normal state or was moving away from the normal state. Either way, at this level of global DNA methylation, the tumor was benign. However, the data may indicate that while these methylation levels are maintained in the benign state, the prostate cell is susceptible to reverting to a cancerous state.

The GM-RESA technology may have the potential to monitor individuals who have become susceptible to getting cancer but yet have not developed the disease. These data indicate that the GM-RESA technology can be exploited as an early warning system to screen for individuals who are susceptible for the development of lung cancer or any other disease.

### Example VI

### Alternative Methyl Sensitive Enzymes Useful for GM-RESA

The present invention relates to the identification of methylation-sensitive enzymes that would serve as biomarkers for global DNA methylation. This example, evaluates several commercially available methylation-sensitive enzymes (out of an estimated total of fifty-three) that are sensitive to the addition of a methyl group at the cytosine base in a CpG dyad.

The number of restriction sites within the human genome believed cleavable by methyl-sensitive enzymes is thought to be greater than 1×10⁶ sites. Each methyl-sensitive enzyme would be expected to serve as a biomarker thereby quantitating the methylation status of the entire genome. In addition, combinations of methyl-sensitive enzymes would also enable a quantitation of the methylation status of the genome. One combination would be associated with one disease state or another combination would be associated with another disease state. Each biomarker, or combination of biomarkers, would be used to monitor the progression of any disease and applied toward diagnosis, prognosis and the monitoring of any therapeutic treatment for any disease. In addition, a single biomarker or combination of would provide the sensitivity and specificity necessary to provide a highly accurate screening tool for any disease.

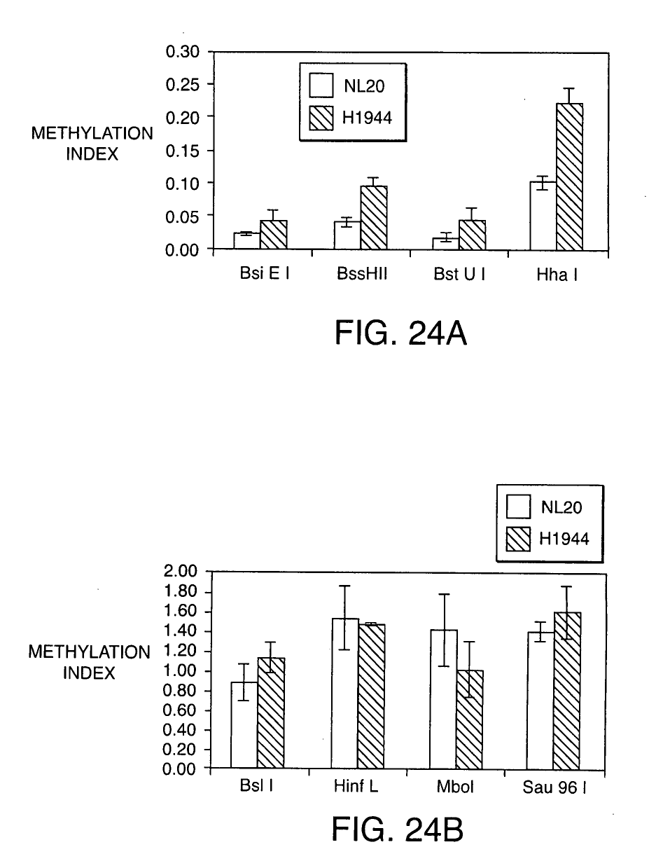

To identify which methyl-sensitive enzymes serve as high quality biomarkers for DNA methylation seventeen (17) commercially available enzymes (AciI, AvaI, BsiEI, BslI, BssHII, BstUI, Fnu4HIV, GlaI, HhaI, HinfI, HinpI, HpyCH4IV, MboI, MwoI, NlaIV, Sau96I and ScRFI) were chosen based on sequence motifs (i.e, for example, comprising one or more CpG dyads within the restriction site) and having a restriction site frequency within the human genome of greater than 10⁶. The methyl-sensitive enzymes were used on the normal lung and the Stage 3B lung cancer cell line in the GM-RESA utilizing the procedure outlined above. The results indicated that seven (7) enzymes AciI, AvaI, HhaI, HinpI, HpyCH4IV, MwoI and NlaIV demonstrated >10% difference between normal lung and the Stage 3B lung cancer cell line (FIG. 24). These data indicated that these methyl sensitive enzyme possessed quantitative traits in the measurement of global DNA methylation in lung cancer. In particular, these seven enzymes plus HpaII would be highly informative biomarkers for global DNA methylation in this disease.

To determine how the seven methyl sensitive enzymes and HpaII behaved when measuring changes in methylation, an experiment using the normal lung cell line DNA mixed with the lung cancer Stage IIIb cell line was performed. Previous calculations using the HpaII methyl sensitive enzyme showed that the amount of methylation in the genome of the normal cell line was 70% and in the Stage IIIb was 40%. Thus, prior to performing the experiment, the two DNAs were mixed in different ratios so that the amount of normal mixed with the cancer cell line varied in increments of 10% from 100% to 0%. In effect, the amount of methylation difference that would be measured at the lowest ratio (90% normal:10% tumor) is 4% (10% of 40% of methylation in the tumor genome in a background of 10% of 70% methylation in normal genome). Thus, for every 10% increase of tumor DNA added, and consequent 10% reduction in normal DNA, there will be an increase of 4% hypomethylation. The analytical sensitivity of GM-RESA is ˜5% so then at this level the assay should detect a difference at the lowest level which should increase with linearity over the entire dilution spectrum.

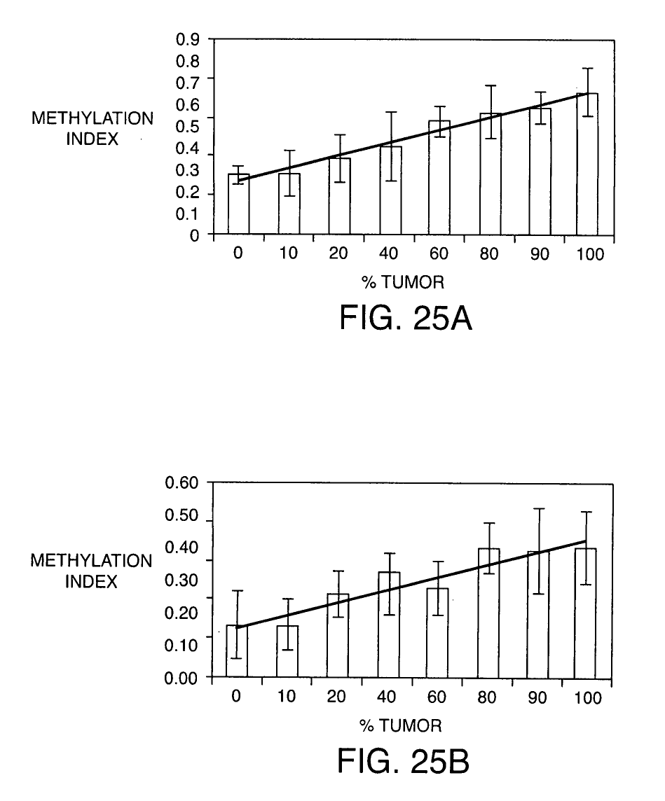

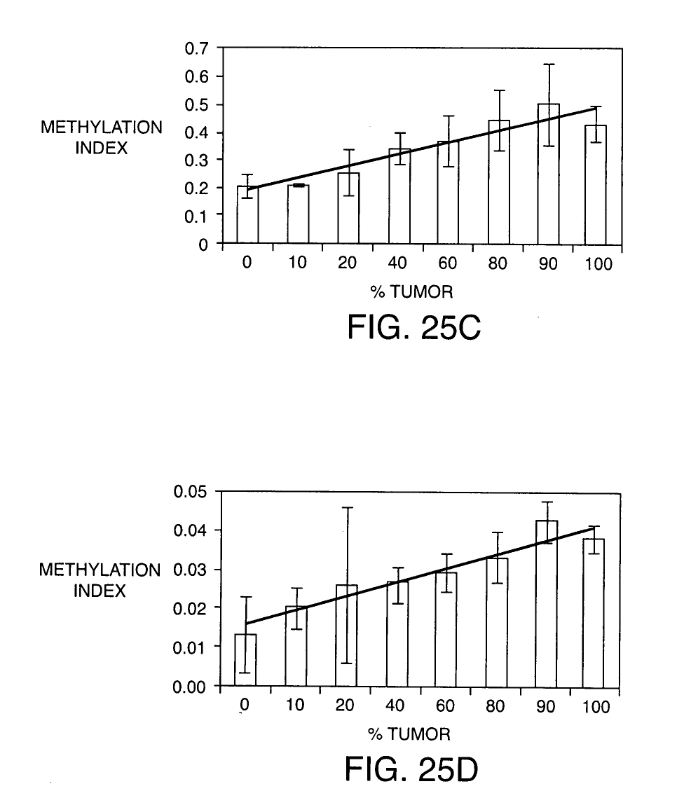

For each methylation sensitive enzyme, 100 ng of each DNA mixture was digested (in triplicate). In another aliquot, 100 ng of each DNA mixture (in triplicate) was digested with MspI for normalization. After end-labeling with biotinylated nucleotides, and measurement of the chemiluminescence, a graph was plotted of luminescence (methyl sensitive enzyme/MspI) versus percent methylation (FIGS. 25A-H). The results showed that for each methyl-sensitive enzyme, a linear increase in hypomethylation was observed between 5 to 10% (depending on the enzyme and the efficiency with which the enzyme was able to digest the DNA to completion). NlaIV and MwoI, showed a linear up to 50% normal:50% tumor ratio toward the lower end indicating that these enzymes were still sensitive at detecting methylation changes and could be applied to lung cancer and other diseases. These data further support that GM-RESA is a highly sensitive assay that can detect changes in global DNA methylation perturbations and it has high value as a screening tool for the diagnosis, prognosis and the monitoring of a therapeutic treatment for any disease.

### Example VII

### Methylation Status of Paired Tumor and Adjacent Normal Lung Tissue Samples

This example evaluates a hypothesis that for an individual to develop lung cancer the methylation status of the lung must have been altered to a hypomethylation state. This hypothesis is consistent with some early changes that have been observed in cancer progression. Although it is not necessary to understand the mechanism of an invention, it is believed that the cells that comprise the lung tissue are primed to proceed from a pre-neoplasia to a neoplasia stage. Those cells that enter the tumor development phase will continue to show changes in methylation either producing an increase in hypomethylation or a greater amount of hypermethylation (gain of methylation) in particular at the CpG islands, which are typically unmethylated. However, those cells that are in the pre-neoplasia stage will still maintain a level of hypomethylation and therefore will be primed to become tumor cells.

Paired tumor (T) and adjacent normal (ND—normal disease) lung tissue samples, collected during surgical resection of the diseased lung from 9 patients with Stage IA or Stage IB lung cancer, were analyzed using GM-RESA to measure the levels of global DNA methylation. The optimized protocol outlined above was utilized.

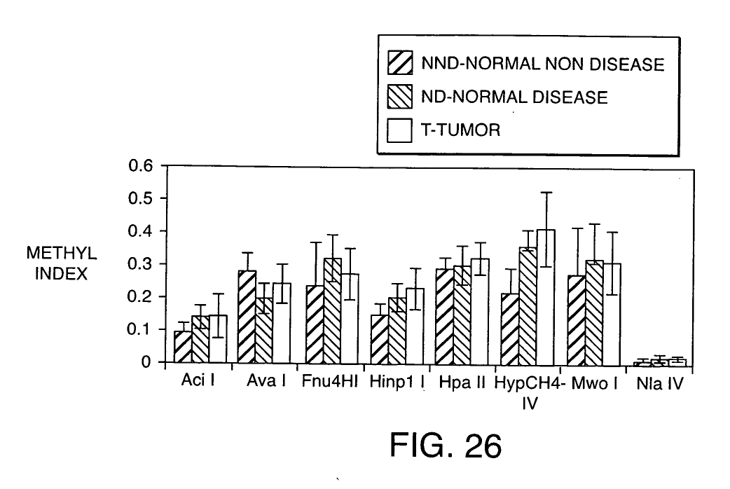

Eight methylation sensitive enzymes (AciI, AvaI, Fnu4HI, Hinpl, HpaII, HpyCh4 IV, MwoI and NlaIV) were applied to the lung tissue DNA from 10 normal controls (NND—normal non-disease) and the paired tumor (T) and adjacent normal (ND—normal disease). The global methylation index was calculated for each sample (luminescence of methyl enzyme/luminescence with MspI) that was treated with a methyl sensitive enzyme. The mean global DNA methylation index was calculated for each group (NND, ND and T to derive a mean on the 10 normal controls and the 9 paired normal and tumor samples and plotted on a graph of methyl index versus enzyme (FIG. 26). A paired T-test was performed to compare NND/ND, NND/T and ND/T to determine which enzyme gave a P-value <0.005 (Table I).

In particular, the results from the paired T-test of NND/ND demonstrate that GM-RESA is sensitive to methylation changes in lung tissues that have already been shown to possess a capacity for developing tumors. Five enzymes, AciI, AvaI, HinpI, HypCH4IV and NlaI, had a p-value of <0.005 (Table I). These enzymes represent a set that provide the sensitivity and specificity needed to utilize GM-RESA as a screening tool for lung cancer.

TABLE I
Paired T-test of normal non-disease versus normal disease, normal non-disease
versus for each of the methyl sensitive enzymes. Tumor and normal disease versus tumor.

| | AciI | AvaI | Fnu4HI | HinpI | HpaII | HypCH4 | MwoI | Nla IV |
|---|---|---|---|---|---|---|---|---|
| Normal non-disease/Normal disease | 3.70E−04 | 2.40E−03 | 6.40E−02 | 1.20E−03 | 5.00E−01 | 5.54E−07 | 2.30E−01 | 2.40E−02 |
| Normal non-disease/Tumor | 1.00E−04 | 1.79E−07 | 2.50E−03 | 1.41E−08 | 1.60E−02 | 1.38E−11 | 2.10E−01 | 1.60E−02 |
| Normal disease/Tumor | 8.60E−01 | 7.30E−03 | 1.90E−01 | 2.00E−02 | 7.60E−02 | 3.00E−02 | 8.80E−01 | 2.60E−02 |

Shaded boxes indicate p < 0.005.

### Example VIII

### Methylation Status of Buccal Cell DNA from Smoking Subjects

In this example, GM-RESA measures changes in global DNA methylation from buccal mucosal cells and compares subjects that have not smoked cigarettes (non-smokers) to those subjects that have smoked cigarettes (smokers).B Buccal scrapes were taken from a group of smokers (N=3) and non-smokers (N=5) to determine whether there was a difference in global DNA methylation assay between the sample populations. Buccal scrapes were taken using a dacron brush, and the DNA was isolated using a Blood and Cell Culture DNA Midi Kit® (Qiagen). The concentration of buccal cell DNA was routinely between approximately 2-4 μg as measured using a UV spectrophotometer GM-RESA was performed using HpaII and MspI.

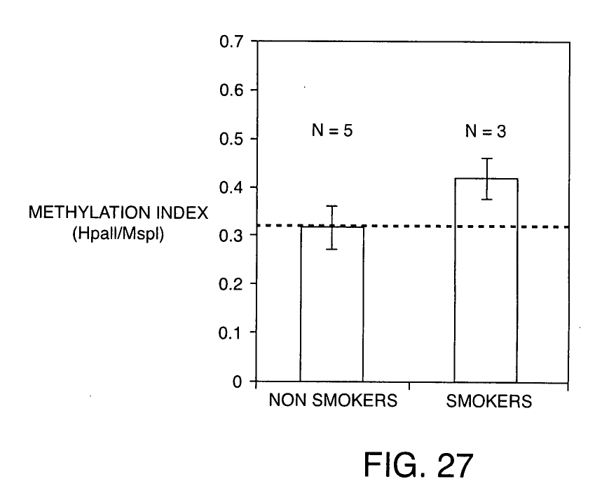

The data showed that the methylation index of the non-smokers was 0.32±0.04 SD and the smoker's methylation index was 0.42±0.04 SD (FIG. 27). The non-smokers possessed a lower methylation index than the smokers but the smokers' methylation index was not as high as the lung cancer cell lines' in particular Stages II to IV (FIG. 14a). The data indicates that the smokers possessed a higher level of global DNA hypomethylation as compared to normal. Although it is not necessary to understand the mechanism of an invention, it is believed that this difference is related to changes in genome integrity. However, the methylation levels were not as low as was observed in the cell lines of the later cancer stages but was similar to the Stage I cell line (0.4±0.04 SD). These changes indicate that the methylation status of the genome had changed and are believed primed to become tumors. Thus, this hypothesis predicts that individuals at-risk for developing of developing lung cancer will have a higher methylation index than normal and thus are more susceptible to developing the disease.

### Example IX

### Methylation Status of Normal Versus Asthma Lung Tissue Samples

In this example, GM-RESA measures the changes in global DNA methylation from lung tissue obtained from individuals with asthma and compares the data to lung tissue from individuals without asthma.

The lung tissue from 5 asthmatic individuals was compared with 3 normal. The DNA was isolated from the lung tissue using the Blood and Cell Culture DNA Midi Kit® (Qiagen). The concentration of the DNA was measured using a UV spectrophotometer. The established protocol outlined above was utilized on the 3 normals and 5 asthmatic individuals.

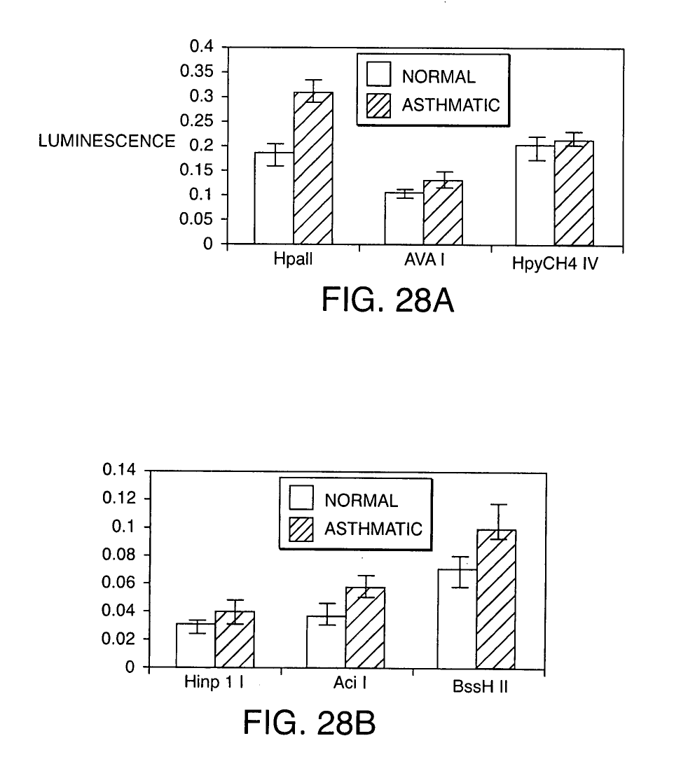

Six methyl sensitive enzymes (AciI, AvaI, BssHII, Hinpl, HpaII and HpyCH4IV) were applied to the asthmatic and normal lung tissue DNAs. For each methyl sensitive enzyme the mean methylation index of the 3 normal was taken and compared against the methylation index of the 5 asthmatic individuals. The resultant data indicated that there was a higher level of global DNA hypomethylation in the asthma samples compared to the normal for all six enzymes. FIG. 28. A paired T-test of normal versus asthma indicated that several enzymes, AciI, BssHII, HinpI, and AviI, had a p-value <0.005 (Table II). These data support the role of hypomethylation in a complex disease such as asthma and indicate the role of this technology as a screening tool for the diagnosis, prognosis and monitoring of treatment in asthma and any other disease.

TABLE II
Paired T-test of normal versus asthma lung DNA. Boxes
highlighted in yellow indicate where p < 0.005.

| | AciI | AvaI | BssHII | HinpI | HpaII | HpyCH4 IV |
|---|---|---|---|---|---|---|
| Normal/ | 0.0007 | 0.03 | 0.0003 | 0.0002 | 0.0004 | 0.2 |
| Asthma | | | | | | |

### Example X

### High-Throughput GM-RESA

In this example, GM-RESA is performed in a 384 well microtiter plate. The present invention contemplates similar methodology that includes microtiter plates comprising, for example, a 96 and/or a 1536 well plate, as well as microfluidic biochips, and maintain the analytical sensitivity that was demonstrated above for a 96 well microtiter plate. In addition, this invention contemplates a reduced amount of material required to perform the assay in a 384 well microtiter plate, which further demonstrates the application of the technology in smaller wells and as such would extend to a microfluidic biochip. Further, the use of lower amounts of each reagent in a 384 well microtiter plate reduces the cost of performing the assay on each sample. Also, as less reagent will be used in the assay so too will less patient material, which will be of value when measuring free circulating DNA in the blood and urine, where amounts can vary from 10 ng to 100 ng.

1. Linearity of End-Labeling Reaction

As was determined above for a 96 well microtiter plate, the set of end-labeling conditions was linear with respect to the amount of DNA ends available in the reaction for a 384 well microtiter plate. An experiment was performed using MspI to digest varying amounts of DNA and measured the incorporation of biotinylated nucleotides after digestion.

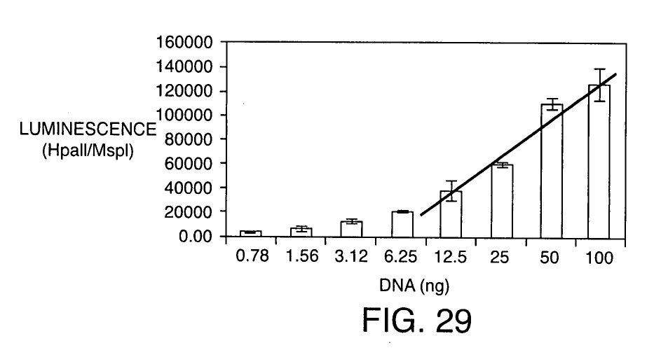

Incorporation of biotinylated nucleotides into the 5′CG overhang of the MspI digested DNA should be proportional to the amount of DNA being assayed. Genomic DNA (100 ng, 50 ng, 25 ng, 12.5 ng, 6.25 ng, 3.125 ng, 1.56 ng and 0.78 ng) was aliquoted (in triplicate) into a 384 well Microfluor® 2 White plate (Thermo Electron) and digested with 10 units of MspI for 2 hours at 37° C. The DNA was end-labeled with 1 unit of Sequenase® and 0.1 μM biotin-dCTP and biotin-dGTP. The DNA was mixed with Reacti-Bind® (Pierce) to adhere it to the surface of the plate. The biotin was detected using neutravidin and the HRP Chemiluminescence kit (HRP) and quantitated by a Wallac Envision 2100 multilabel reader (Perkin Elmer). The results indicated that there was a linear relationship between the amount of DNA digested and the amount of biotin incorporated in an end-fill reaction, which ranged from 12.5 to 100 ng (FIG. 29). For DNA concentrations that were less than 12.5 ng the background was observed to be high with luminescence levels similar to the HpaII result. Although it is not necessary to understand the mechanism of an invention, it is believed that this may explain why the assay was non-linear below 12.5 ng of DNA.

2. Normalization of Methylation Sensitive Restriction Enzyme Reaction

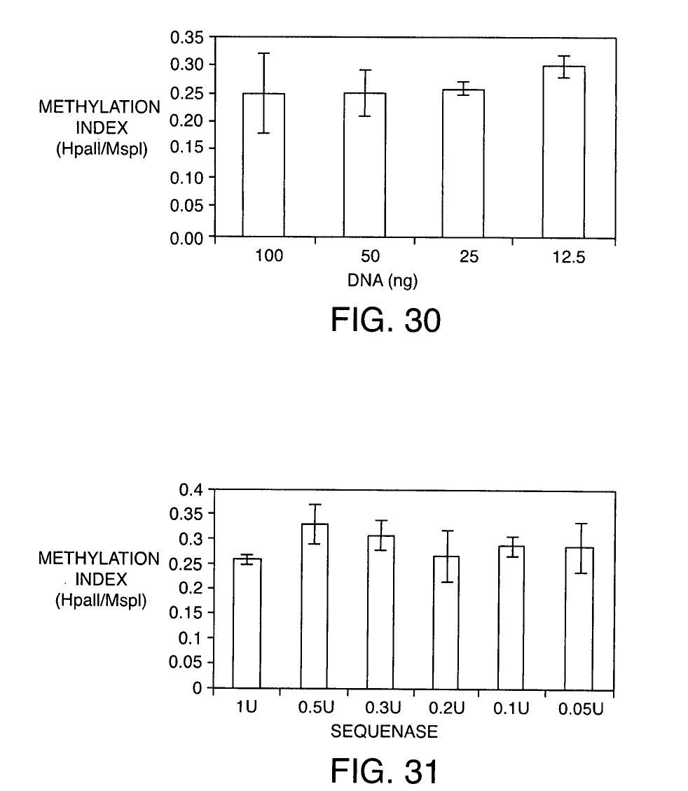

A normalization step was performed by using HpaII (i.e., for example, a methyl sensitive enzyme) and MspI (i.e., for example, a methyl insensitive enzyme) to digest 100 ng, 50 ng, 25 ng and 12.5 ng genomic DNA that had been aliquoted (in triplicate) into a 384 well Microfluor® 2 white plate (Thermo Electron). Ten units of HpaII and 10 units of MspI were used to digest the various concentrations of DNA for 2 hours at 37° C. The DNAs were end-labeled with 1 unit of Sequenase® and 0.1 μM biotin-dCTP and biotin-dGTP. The biotin was detected using neutravidin (Pierce) and the HRP Chemiluminescence kit (HRP) and quantitated by a Wallac Envision 2100 multilabel reader (Perkin Elmer). The chemiluminescence values from the HpaII digestions were divided by the MspI values to generate a normalized HpaII result (FIG. 30). The results indicated that there was a good level of normalization of the HpaII chemiluminescence values (methylation index 0.25-0.3) across a broad spectrum of DNA concentrations. These data indicate that the normalization step using MspI in a 384 well microtiter plate provides the appropriate means to reduce the errors from inter-sample variation of genomic DNA concentrations.

3. Optimization of DNA Sample Size

From the above two experiments it was determined that the minimum amount of DNA that could be used to provide reproducible results in a 384 microtiter plate was approximately 25 ng of genomic DNA. In addition, if a patient sample was low, less DNA could be used without too much compromise in data quality.

4. Titration of Sequenase® in the End-Fill Reaction

The efficiency of the DNA polymerase Sequenase® in an end-fill reaction was performed in a 384 microtiter plate. The amount of Sequenase® was varied but the concentrations of biotinylated nucleotides and the incubation time was fixed as described below:

Incubation time (at 37° C.): 30 minutes;

Amount of biotin-dCTP and biotin-dGTP: 0.1 μM; and

Amount of Sequenase®: 1.0 units, 0.5 units, 0.3 units, 0.2 units, 0.1 units, and 0.05 units.

In addition, the experiment was performed in triplicate using 25 ng of genomic DNA, which had been digested with HpaII and MspI. The results indicated that the amount of Sequenase added into each experiment produced similar methylation indices when using a high amount (1.0 unit as recommended by the manufacturer or a low amount 0.05 units) (FIG. 31). 0.1 unit of Sequenase® was selected as the optimum amount in the end-labeling reaction, which was similar to the amount required to end-label the digested DNA in the 96 well microtiter plate.

5. Analytical Sensitivity

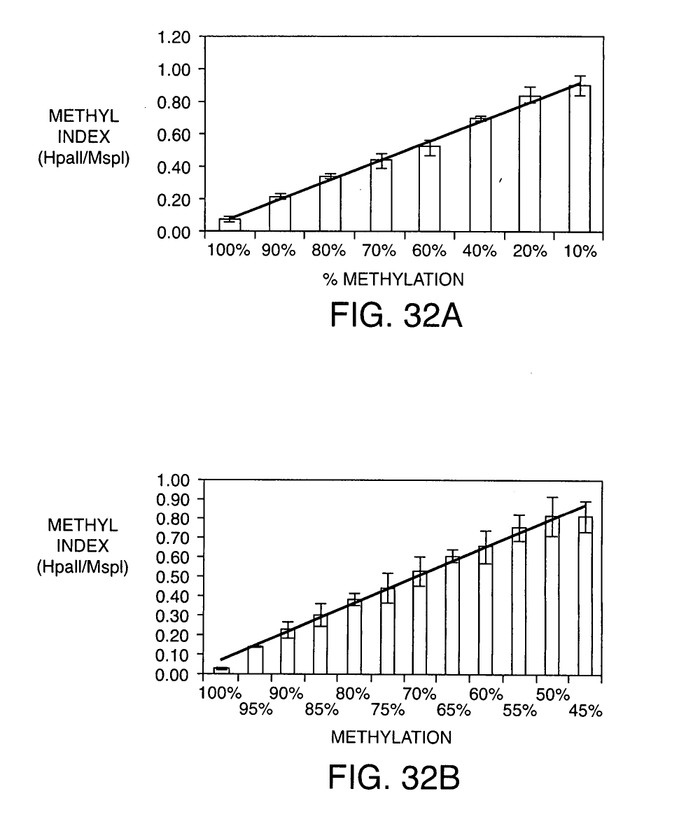

The analytical sensitivity of GM-RESA in a 384 well microtiter plate was determined exactly the same way as described for the 96 well mictrotiter plate. Lambda DNA was used as the test DNA in the 384 well microtiter plate. To determine the analytical sensitivity of GM-RESA the entire genome of Lambda DNA was methylated using SssI; a bacterial enzyme that methylates all cytosine residues within a CpG dyad. The methylated Lambda DNA was mixed with unmethylated Lambda DNA so that the increments of percent methylation (mixed with unmethylated) increased every 10% from 100% to 10%. Twelve-and-a-half nanograms of each DNA mix was placed directly into a 384 Microfluor® 2 White plate (in triplicate) and digested with the methyl sensitive enzyme HpaII and in another aliquot the DNA was digested with the methyl insensitive enzyme MspI. After end-labeling with biotinylated nucleotides and measurement of the chemiluminescence, a graph was plotted of Methyl Index (HpaII/MspI) versus percent methylation (FIG. 32A). The results showed a linear 10% decrease in methylation of the Lambda DNA from 100% to 10% indicating that the analytical sensitivity under these experimental conditions was 10%. To refine the level of analytical sensitivity the assay was repeated except that the increments of percent methylation (mixed with unmethylated) DNA increased every 5% from 100% to 50% (FIG. 32B). The results showed a linear 5% decrease in methylation of the Lambda DNA from 100% to 50% and that the analytical sensitivity was ˜5%, which was similar to the analytical sensitivity of the 96 well microtiter plate. These data showed that GM-RESA was highly sensitive at detecting changes in global DNA methylation perturbations in a 384 microtiter plate.

We claim:

1. A method, comprising:
a) providing;
i) a nucleic acid comprising at least one CG dinucleotide, wherein the cytosine is methylated;
ii) a primer set comprising a forward primer and a reverse primer, wherein said forward primer will hybridize to a region of said nucleic acid comprising said CG dinucleotide;
iii) a methylation specific restriction enzyme capable of cleaving a methylation restriction site provided said site is non-methylated;
b) treating said nucleic acid with said methylation specific restriction enzyme so as to create a digest comprising fragments, wherein at least one of said fragments comprises said region comprising said CG dinucleotide; and
c) introducing said forward and reverse primers under conditions such that said forward primer hybridizes to said region comprising CG dinucleotide and a portion of said fragment is amplified so as to create amplified product.

2. The method of claim 1, wherein said forward primer is of the formula: Z-C-G-Xₙ—Yᵣ, wherein X and Y are different nucleic acid bases, wherein Z is any nucleic acid base or nothing, and wherein n and r are independently whole numbers between 2 and 5, wherein the primer as a whole has fewer than seven cytosines and fewer than seven guanosines.

3. The method of claim 1, wherein said forward primer is of the formula: Z-C-G-Xₙ-Z-Yᵣ, wherein X and Y are different nucleic acid bases, wherein Z is any nucleic acid base or nothing, and wherein n and r are independently whole numbers between 2 and 5, wherein the primer as a whole has fewer than seven cytosines and fewer than seven guanosines.

4. The method of claim 1, wherein said forward primer is of the formula: Z-C-G-Z-Xₙ—Yᵣ, wherein X and Y are different nucleic acid bases, wherein Z is any nucleic acid base or nothing, and wherein n and r are independently whole numbers between 2 and 5, wherein the primer as a whole has fewer than seven cytosines and fewer than seven guanosines.

5. The method of claim 1, wherein said forward primer is of the formula: Z-C-G-Xₙ—Yᵣ-Z wherein X and Y are different nucleic acid bases, wherein Z is any nucleic acid base or nothing, and wherein n and r are independently whole numbers between 2 and 5, wherein the primer as a whole has fewer than seven cytosines and fewer than seven guanosines.

6. The method of claim 1, wherein said forward primer is of the formula: Xₙ—C-G-Yᵣ, wherein X and Y are different nucleic acid bases and wherein n and r are independently whole numbers between 2 and 6, wherein the primer as a whole has fewer than seven cytosines and fewer than seven guanosines.

7. The method of claim 1, wherein said forward primer is of the formula: Xₙ—Yᵣ—C-G-Xₙ—Yᵣ wherein X and Y are different nucleic acid bases and n and r are independently whole numbers between 1 and 3, wherein the primer as a whole has fewer than seven cytosines and fewer than seven guanosines.

8. The method of claim 1, wherein said forward primer is of the formula: Xₙ-Z-Yᵣ—C-G-Xₙ-Z-Yᵣ, wherein X and Y are different nucleic acid bases, wherein Z is any nucleic acid base or nothing, and n and r are independently whole numbers between 1 and 3, wherein the primer as a whole has fewer than seven cytosines and fewer than seven guanosines.

9. The method of claim 1, wherein said forward primer is of the formula: Z_{q}-Xₙ—Yᵣ—C-G-Z_{q}-Xₙ—Yᵣ wherein X and Y are different nucleic acid bases, wherein Z is any nucleic acid base, and wherein q, n and r are independently whole numbers between 0 and 3, wherein the primer as a whole has fewer than seven cytosines and fewer than seven guanosines.

10. The method of claim 1, wherein said forward primer is of the formula: Z_{q}-Xₙ—Yᵣ—C-G-Z_{q}, wherein X and Y are different nucleic acid bases, wherein Z is any nucleic acid base, and wherein q, n and r are independently whole numbers between 0 and 3, wherein the primer as a whole has fewer than seven cytosines and fewer than seven guanosines.

11. The method of claim 1, wherein said forward primer is a 10-mer and comprises methylation sensitive restriction site.

12. The method of claim 1, wherein said forward primer and said reverse primer is selected from the forward and reverse primers of Table 1.

13. A method, comprising:
a) providing;
i) a nucleic acid comprising at least one CpG Island, wherein said Island comprises at least one methylation restriction site;
ii) at least one primer set comprising a forward primer and a reverse primer, wherein said forward primer is substantially homologous to said restriction site;
iii) a methylation specific restriction enzyme capable of cleaving said methylation restriction site provided said site is methylated;
iv) a methylation sensitive restriction site capable of cleaving said methylation restriction site provided said site is non-methylated;
v) a methylation insensitive restriction enzyme capable of cleaving said restriction site whether said restriction site is methylated or non-methylated; and
b) contacting a first aliquot of said DNA with said methylation specific restriction enzyme to generate a first DNA fragment that is substantially homologous to said primer set;
c) contacting a second aliquot of said DNA with said methylation sensitive restriction enzyme to generate a second DNA fragment that is substantially homologous to said primer set; and
d) contacting a third aliquot of said DNA with said methylation insensitive restriction enzyme to generate a third DNA fragment that is substantially homologous to said primer set.

14. The method of claim 13, further comprising end-labeling said first, second and third DNA fragments.

15. The method of claim 13, further comprising amplifying said first, second and third DNA fragments to generate cDNA.

16. The method of claim 15, further comprising separating said cDNA under conditions such that said methylated restriction site is identified.

17. The method of claim 13, wherein said nucleic acid is selected from the group consisting of free circulating DNA and genomic DNA.

18. A GM/RESA method, comprising:
a) providing;
i) isolated genomic DNA, wherein said DNA comprises at least one restriction site, wherein said restriction site comprises a cytosine residue capable of a 5′-methylation;
ii) a methylation sensitive restriction enzyme;
iii) a methylation insensitive restriction enzyme;
iv) a biotinylated nucleotide selected from the group consisting of cytosine, guanidine, thymidine and adenine;
v) a biotin-specific fluorescent marker; and
b) contacting said methylation sensitive restriction enzyme with a first aliquot of said genomic DNA to create a first plurality of restriction fragments;
c) contacting said methylation insensitive restriction enzyme with a second aliquot of said genomic DNA thereby creating a second plurality of restriction fragments;
d) incorporating said biotinylated nucleotide into said first and second plurality of restriction fragments thereby creating a first and second plurality of biotinylated restriction fragments; and
e) detecting said incorporated biotin in said restriction fragments under conditions such that a sample methylation index is calculated.

19. The method of claim 18, wherein said isolated genomic DNA is obtained from a patient.

20. The method of claim 18, further comprising step (f) comparing said sample methylation index with a normal methylation index.

21. The method of claim 20, wherein said comparison identifies said calculated methylation index as representing a hypomethylation state.

22. The method of claim 21, wherein said hypomethylation state identifies said patient is at risk for a disease.

23. The method of claim 21, wherein said hypomethylation state identifies said patient as having a disease.

24. The method of claim 21, wherein said detecting of said incorporated biotin is performed using a biotin-specific fluorescent marker.
